(19) United States
(12) Patent Application Publication
(10) Pub. No.: US 2020/0339686 A1
(43) Pub. Date: Oct. 29, 2020

(54) BISPECIFIC ANTIBODY THAT BINDS CD3 AND ANOTHER TARGET

(71) Applicant: LAKEPHARMA, INC., SO. San Francisco, CA (US)

(72) Inventors: Aaron SATO, SO. San Francisco, CA (US); Jordon WANG, SO. San Francisco, CA (US); Kexin HUANG, SO. San Francisco, CA (US)

(21) Appl. No.: 16/962,180

(22) PCT Filed: Jan. 15, 2019
(86) PCT No.: PCT/US2019/013711

§ 371 Date: Jul. 14, 2020

Related U.S. Application Data
(60) Provisional application No. 62/618,019, filed on Jan. 16, 2018.

(51) Int. Cl. C07K 16/28; A61K 47/68

(52) U.S. Cl. CPC C07K 16/2809; A61K 47/6803; A61K 47/6851; A61K 2039/505; C07K 2317/24; C07K 2317/52; C07K 2317/31

(57) ABSTRACT

Bispecific anti-cluster of differentiation 3 (CD3) antibodies and methods of using the same.

### CROSS-REFERENCE TO RELATED APPLICATIONS

This application claims priority to U.S. Provisional Patent Application No. 62/618,019, filed Jan. 16, 2018, which is incorporated by reference herein in its entirety, including any drawings.

### FIELD OF THE INVENTION

The present invention relates to bispecific anti-cluster of differentiation 3 (CD3) antibodies and methods of using the same.

### BACKGROUND

Cell proliferative disorders, such as cancer, are characterized by the uncontrolled growth of cell subpopulations. They are the leading cause of death in the developed world and the second leading cause of death in developing countries, with over 12 million new cancer cases diagnosed and 7 million cancer deaths occurring each year. The National Cancer Institute estimates that greater than half a million Americans will die of cancer in 2018, accounting for nearly one out of every four deaths in the country. As the elderly population has grown, the incidence of cancer has concurrently risen, as the probability of developing cancer is more than two-fold higher after the age of seventy. Cancer care thus represents a significant and ever-increasing societal burden.

Longstanding approaches to cancer treatment include chemotherapy, radiation therapy, and surgery to remove solid tumors. Recently, bispecific antibody-based immunotherapies have been developed. Such bispecific antibodies are capable of simultaneously binding cell surface antigens on cytotoxic cells and tumor cells, with the intent that the bound cytotoxic cell will destroy the bound tumor cell. Existing bispecific antibodies currently undergoing clinical trials for treating cancer are limited by their short half-lives and/or variable efficacy. Thus, there is an unmet need in the field for the development of effective bispecific antibodies for use in cancer treatment.

### SUMMARY

The present invention relates to humanized anti-cluster of differentiation 3 (“CD3”) bispecific antibodies and methods of using the same. Some embodiments provide a bispecific antibody that binds to CD3 and another antigen, wherein the bispecific antibody comprises a first heavy chain binding domain and a second heavy chain binding domain, the first heavy chain binding domain comprising a V_{H} comprising one or more of: a CDR-H1 comprising a polypeptide comprising SEQ ID NO: 5; a CDR-H2 comprising a polypeptide comprising an amino acid sequence of SEQ ID NO: 6; and a CDR-H3 comprising a polypeptide comprising an amino acid sequence of SEQ ID NO: 7; and the second heavy chain binding domain comprising a V_{H} comprising one or more of: a CDR-H1 comprising a polypeptide comprising an amino acid sequence of one or more of SEQ ID NOs: 20-21; a polypeptide comprising a CDR-H2 comprising an amino acid sequence of one or more of SEQ ID NOs: 22-23; and a polypeptide comprising a CDR-H3 comprising an amino acid sequence of one or more of SEQ ID NOs: 24-25. In some embodiments, the V_{H} comprises a polypeptide comprising an amino acid sequence of at least one of SEQ NOs: 1-4 and/or SEQ ID NOs: 15-19.

Some embodiments provide a bispecific antibody that binds to CD3 and another antigen, wherein the bispecific antibody comprises a first light chain binding domain and a second light chain binding domain, the first light chain binding domain comprising a V_{L} comprising one or more of: a CDR-L1 comprising a polypeptide comprising an amino acid sequence of SEQ ID NO: 12; a CDR-L2 comprising a polypeptide comprising an amino acid sequence of SEQ ID NO: 13; and a CDR-L3 comprising a polypeptide comprising an amino acid sequence of SEQ ID NO: 14; and the second light chain binding domain comprising a V_{L} comprising one or more of: a CDR-L1 comprising a polypeptide comprising an amino acid sequence of SEQ ID NO: 28; a CDR-L2 comprising a polypeptide comprising an amino acid sequence of SEQ ID NO: 29; and a CDR-L3 comprising a polypeptide comprising an amino acid sequence of SEQ ID NO: 30. In some embodiments, the V_{L} comprises a polypeptide comprising an amino acid sequence of at least one of SEQ NOs: 8-11 and/or SEQ ID NOs: 26-27.

Some embodiments provide a bispecific antibody that binds to CD3 and another antigen, wherein the bispecific antibody comprises a first heavy chain binding domain comprising at least one polypeptide comprising an amino acid sequence of at least one of SEQ NOs: 1-4 and a second heavy chain binding domain comprising at least one polypeptide comprising an amino acid sequence of at least one of SEQ ID NOs: 15-19 and a first light chain binding domain comprising at least one polypeptide comprising an amino acid sequence of at least one of SEQ NOs: 8-11 and a second light chain binding domain comprising at least one polypeptide comprising an amino acid sequence of one of at least one of SEQ ID NOs: 26-27. Some embodiments provide a bispecific antibody that binds to CD3 and another antigen, wherein the bispecific antibody comprises a first heavy chain binding domain comprising one or more polypeptides comprising an amino acid sequence having at least 95% sequence identity to one or more of SEQ NOs: 1-4 and a second heavy chain binding domain comprising one or more polypeptides comprising an amino acid sequence having at least 95% sequence identity to one or more of SEQ ID NOs: 15-19, a first light chain binding domain comprising a polypeptide comprising an amino acid sequence having at least 95% sequence identity to one or more of SEQ NOs: 8-11 and a second light chain binding domain comprising a polypeptide comprising an amino acid having at least 95% sequence identity to one of SEQ ID NOs: 26-27.

Some embodiments provide a bispecific antibody that binds to CD3 and another antigen, wherein the bispecific antibody comprises at least one polypeptide comprising an amino acid sequence of any one or more of SEQ ID NOs: 31-54 or comprises at least one polypeptide comprising an amino acid sequence of any one or more of sequences or Figures set forth in this specification.

Some embodiments provide a bispecific antibody that comprises more than one polypeptide comprising a combination of more than one amino acid sequence set forth in the specification. For example, without limitation, some embodiments provide a bispecific antibody comprising a combination of more than one polypeptide comprising an amino acid sequence of one or more of SEQ ID NO: 42, SEQ ID NO: 43, SEQ ID NO: 44. SEQ ID NO: 45, SEQ ID NO: 46, and/or SEQ ID NO: 47. Some embodiments provide a bispecific antibody comprising more than one polypeptide comprising an amino acid sequence as set forth in SEQ ID NO: 42, SEQ ID NO: 43, and SEQ ID NO: 42. Some embodiments provide a bispecific antibody comprising more than one polypeptide comprising an amino acid sequence as set forth in SEQ ID NO: 44, SEQ ID NO: 45, and SEQ ID NO: 42. Some embodiments provide a bispecific antibody comprising more than one polypeptide comprising an amino acid sequence as set forth in SEQ ID NO: 47, SEQ ID NO: 46, and SEQ ID NO: 42. Some embodiments provide a bispecific antibody comprising more than one polypeptide comprising an amino acid sequence as set forth in SEQ ID NO: 47, SEQ ID NO: 45, and SEQ ID NO: 42. Some embodiments provide a bispecific antibody comprising more than one polypeptide comprising more than one amino acid sequence having at least 95%, 96%, 97%, 98%, or 99% to one or more of SEQ ID NO: 42, SEQ ID NO: 43, SEQ ID NO: 44. SEQ ID NO: 45, SEQ ID NO: 46, and/or SEQ ID NO: 47.

In some embodiments, the another antigen is a cell surface antigen. In some embodiments, the cell surface antigen is a tumor antigen. In some embodiments, the tumor antigen is selected from the group consisting of CD20; FcRH5 (Fc Receptor-like 5); HER2; LYPD1; Ly6G6D (lymphocyte antigen 6 complex, locus G61); Ly6-D, MEGT1); PMEL17 (silver homolog; SILV; D12S53E; PMEL17; (SI); (SIL); ME20; gp100); Ly6E (lymphocyte antigen 6 complex, locus E; Ly67, RIG-E, SCA-2, TSA-1); CD19; CD33; CD22 (B-cell receptor CD22-B isoform); CD79a (CD79A, CD79a, immunoglobulin-associated alpha; BMPR1 B (bone morphogenetic protein receptor-type IB); CD79b (CD79B, CD790, 1 Gb (immunoglobulin-associated beta), B29); EDAR (Ectodysplasin A Receptor); GFRA1 (GDNF-Ral); MRP4 (Multidrug Resistance Protein 4); RET; STEAP1 (six transmembrane epithelial antigen of prostate); TENB2 (putative transmembrane proteoglycan); E16 (LAT1, SLC7A5); 0772P (CA125, MUC16); MPF (MPF, MSLN, SMR, megakaryocyte potentiating factor, mesothelin); Napi2b (NAPI-2B, NPTIIb, SLC34A2, solute carrier family 34 (sodium phosphate), member 2, type II sodium-dependent phosphate transporter 3b); Sema 5b; PSCA hlg (2700050C12Rik, C530008016Rik, RIKEN cDNA 2700050C12, RIKEN cDNA 2700050C12 gene); ETBR (Endothelin type B receptor); MSG783 (RNF124, hypothetical protein FLJ20315); STEAP2; TrpM4 (BR22450, FLJ20041, TRPM4, TRPM4B, transient receptor potential cation channel, subfamily M, member 4); CRIPTO (CR, CR1, CRGF, CRIPTO, TDGF1, teratocarcinoma-derived growth factor); CD21 (CR2 (Complement receptor 2) or C3DR (C3d/Epstein Barr virus receptor) or Hs.73792); FcRH2 (IFGP4, IRTA4, SPAP1A (SH2 domain containing phosphatase anchor protein 1a), SPAP1B, SPAPIC); NCA; MDP; IL20Ra; Brevican; EphB2R; ASLG659; PSCA; GEDA; BAFF-R (B cell-activating factor receptor, BLyS receptor 3, BR3); CXCR5 (Burkitt's lymphoma receptor 1; HLA-DOB (Beta subunit of MHC class II molecule); P2X5 (Purinergic receptor P2X ligand-gated ion channel 5; CD72 (B-cell differentiation antigen CD72, Lyb-2); LY64 (Lymphocyte antigen 64 (RP105), type I membrane protein of the leucine rich repeat (LRR) family); FcRH1 (Fc receptor-like protein 1); IRTA2 (Immunoglobulin superfamily receptor translocation associated 2); TMEFF 1; TMEM46 (shisa homolog 2 (Xenopus laevis); SHISA2); LGR5 (leucine-rich repeat-containing G protein-coupled receptor 5; GPR49, GPR67); LY6K (lymphocyte antigen 6 complex, locus K; LY6K; HSJ001348; FLJ35226); GPR19 (G protein-coupled receptor 19; Mm 4787); GPR54 (KISS1 receptor; KISSIR; GPR54; HOT7T175; AXOR12); ASPHDI (aspartate beta-hydroxylase domain containing 1; LOC253982); Tyrosinase (TYR; OCAIA; OCAlA; tyrosinase; SHEP3); TMEM118 (ring finger protein, transmembrane 2; RNFT2; FLJ14627); GPR172A (G protein-coupled receptor 172A; GPCR41; FLJ11856; D15Ertd747e); GPC3 (Glypican 3); CLL1 (C-Type Lectin-like molecule 1); B7-H4 (B7x; B7S1); RNF43 (Ring finger protein 43); CD70; CXORF61 (Chromosome X open reading frame 61); HAVCR1; Epiregulin; Amphiregulin; EGFR; EGFR-L858R; EGFR-L861Q; EGFR-G719A; EGFR-G719S; EGFR-G719C; EGFR-T790M; EGFR-S768; adipophilin; AIM-2; ALDHIAI; alpha-actinin-4; alpha-foetoprotein; ARTC1; B-RAF; BAGE-1; BCLX (L); BCR-ABL fusion protein (b3a2); beta-catenin; BING-4; CALCA; CASP-5; CASP-8; CD45; Cdc27; CDK4; CDKN2A; CEA; CLPP; COA-1; CPSF; Cw6; cyclin D1; Cyclin-A1; dek-can fusion protein; DKK1; DR1; DR13; EFTUD2; Elongation factor 2; ENAH (hMena); EpCAM; EphA3; ETV6-AML1 fusion protein; EZH2; FLT3-ITD; FN1; G250; MN; CAIX; GAGE-1; 2; 8; GAGE-3; 4; 5; 6; 7; glypican-3; GnTVf, gp100/Pmel17; GPNMB; HERV-K-MEL; hsp70-2; IDO1; IGF2B3; IL13Ralpha2; Intestinal carboxyl esterase; K-ras; Kallikrein 4; KIF20A; KK-LC-1; KM-HN-1; LAGE-1; LDLR-fucosyltransferaseASfusion protein; Lengsin; M-CSF; MAGE-A1; MAGE-A10; MAGE-A12; MAGE-A2; MAGE-A3; MAGE-A4; MAGE-A6; MAGE-A9; MAGE-C1; MAGE-C2; mammaglobin-A; MART2; MCSP; mdm-2; MEl; Melan-A/MART-1; Meloe; MMP-2; MMP-7; MUC1; MUC5AC; mucin; MUM-if, MUM-2; MUM-3; Myosin class I; N-ras; NA88-A; neo-PAP; NFYC; NY-BR-1; NY-ESO-1/LAGE-2; OAl; OGT; OS-9; p53; PAP; PAX5; PBF; pml-RARalpha fusion protein; PRAME; PRDX5; PSMA; PTPRK; RAB38/NY-MEL-1; RAGE-1; RBAF600; RGS5; RhoC; RNF43; RU2AS; SAGE; secernin 1; SIRT2; SNRPD1; SOX10; Sp17; SSX-2; SSX-4; STEAP1; survivin; SYT-SSX1 or -SSX2 fusion protein; TAG-1; TAG-2; Telomerase; TGF-betaRII; TRAG-3; Triosephosphate isomerase; TRP-1/gp75; TRP-2; TRP2-INT2; tyrosinase; VEGF; WTi; XAGE-1b/GAGED2a; and SLC35D3. In some embodiments, the tumor antigen is selected from the group consisting of CD20, FcRH5, HER2, LYPD1, LY6G6D, PMEL17, LY6E, CD19, CD33, CD22, CD79A, CD79B, EDAR, GFRA1, MRP4, RET, Steap1, and TenB2. In some embodiments, the antigen is Epcam, PSMA, BCMA, or ROR1.

In some embodiments, the bispecific antibody is an immunoconjugate comprising any one of the preceding anti-CD3 antibodies conjugated to a cytotoxic agent. In some embodiments the bispecific antibody comprises a composition. In some embodiments, the composition further comprises a pharmaceutically acceptable carrier, excipient, or diluent. In some embodiments, the composition is a pharmaceutical composition.

In some embodiments, the bispecific antibody is a full length antibody. In some embodiments, the bispecific antibody is an IgA, an IgD, an IgE, an IgG, or an IgM antibody. In some embodiments, the anti-CD3 antibody is an IgG antibody (e.g., an IgG1, IgG2, or IgG3 antibody).

In some embodiments, the bispecific antibody is an antibody fragment. In some embodiments, the bispecific antibody is an Fv fragment, a Fab fragment, a F(ab′)₂ fragment, a Fab′ fragment, an Fab′-SH, an scFv (sFv) fragment, and an scFv-Fc fragment. In some embodiments, the bispecific antibody is an scFv fragment.

In some embodiments, the bispecific antibody is monoclonal, human, humanized, or chimeric.

In some embodiments, the bispecific antibody further comprises an Fc region. In some embodiments, the bispecific antibody comprises one or more heavy chain constant domains, wherein the one or more heavy chain constant domains are selected from a first CH1 domain, a first CH2 domain, a first CH3 domain, a second CH1 domain, a second CH2 domain, and a second CH3 domain. In some embodiments, one or more heavy constant chain domains are paired with another heavy chain constant domain.

In some embodiments, the bispecific antibody further comprises an aglycosylation site mutation. In some embodiments, the mutation reduces effector function. In some embodiments, the mutation is a substitution mutation.

In some embodiments, the invention features an isolated nucleic acid that encodes any of the bispecific antibodies disclosed herein. In some embodiments, the nucleic acid comprises one or more of the nucleic acids set forth in SEQ ID NOs: 55-63. In some embodiments, the nucleic acid comprises any combination of the nucleic acids set forth in SEQ ID NOs: 51-54. In some embodiments, the nucleic comprises one or more nucleic acids. In some embodiments, the nucleic acid comprises two or more nucleic acids set forth in SEQ ID NO: 55 and one or more nucleic acid set forth in SEQ ID NO: 56. In some embodiments, the nucleic acid comprises one or more nucleic acid set forth in SEQ ID NO: 55, one or more nucleic acid set forth in SEQ ID NO: 57, and one or more nucleic acid set forth in SEQ ID NO: 58. In some embodiments, the nucleic acid comprises one or more nucleic acid set forth in SEQ ID NO: 55, one or more nucleic acid set forth in SEQ ID NO: 59, and one or more nucleic acid set forth in SEQ ID NO: 60. In some embodiments, the nucleic comprises one or more nucleic acid set forth in SEQ ID NO: 55, one or more nucleic acid set forth in SEQ ID NO: 58, and one or more nucleic acid set forth in SEQ ID NO: 60.

Some embodiments comprise a vector for expressing any of the bispecific antibodies provided herein. Some embodiments comprise a host cell comprising a vector expressing any of the bispecific antibodies provided herein. In some embodiments, the host cell is a bacterial cell, a fungal cell, or a mammalian cell. In some embodiments, the host cell is a mammalian cell. In some embodiments, the host cell is a Saccharomyces cerevisiae cell or Chinese hamster ovary (CHO) cell. In some embodiments, the host cell is a prokaryotic cell. In some embodiments, the host cell is an E. coli cell. Some embodiments are drawn to a method of producing any of the bispecific antibodies provided herein, the method comprising culturing the host cell that produces the bispecific antibody and recovering the bispecific antibody from the host cell or the culture medium.

In some aspects, any one of the bispecific antibodies can be used as a medicament. In some embodiments, any one of the bispecific antibodies can be for use in treating or delaying progression of a cell proliferative disorder or an autoimmune disorder in a subject in need thereof. In some embodiments, any the bispecific antibodies can be for use in enhancing or decreasing immune function in a subject having a cell proliferative disorder or an autoimmune disorder.

Some embodiments provide a method of treating or delaying the progression of a cell proliferative disorder or an autoimmune disorder in a subject in need thereof, the method comprising administering to the subject an effective amount any one of the preceding bispecific antibodies provided herein. In another aspect, the invention features a method of enhancing or decreasing immune function in a subject having a cell proliferative disorder or an autoimmune disorder, the method comprising administering to the subject any one of the bispecific antibodies provided herein.

In some embodiments, the bispecific antibody binds to (a) a CD3 molecule located on an immune effector cell and (b) a second biological molecule located on a target cell other than the immune effector cell. In some embodiments, the anti-CD3 antibody activates or decreases the immune effector cell following binding to (a) and (b). In some embodiments, the activated immune effector cell is capable of exerting a cytotoxic effect and/or an apoptotic effect on the target cell.

In some embodiments, the bispecific antibody is administered to the subject in a dosage of about 0.01 mg/kg to about 10 mg/kg. In some embodiments, the bispecific antibody is administered to the subject in a dosage of about 0.1 mg/kg to about 10 mg/kg. In some embodiments, the bispecific antibody is administered to the subject in a dosage of about 1 mg/kg. In some embodiments, the bispecific antibody is administered subcutaneously, intravenously, intramuscularly, topically, orally, transdermally, intraperitoneally, intraorbitally, by implantation, by inhalation, intrathecally, intraventricularly, or intranasally. In some embodiments, the bispecific antibody is administered subcutaneously. In some embodiments, the bispecific antibody is administered intravenously.

In any of the uses or methods set forth herein, the cell proliferative disorder can be cancer. In some embodiments, the cancer is selected from the group consisting of breast cancer, colorectal cancer, non-small cell lung cancer, non-Hodgkin's lymphoma (NHL), B cell lymphoma, B cell leukemia, multiple myeloma, renal cancer, prostate cancer, liver cancer, head and neck cancer, melanoma, ovarian cancer, mesothelioma, glioblastoma, germinal-center B-cell-like (GCB) DLBCL, activated B-cell-like (ABC) DLBCL, follicular lymphoma (FL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), Waldenstrom macroglobulinemia (WM), central nervous system lymphoma (CNSL), Burkitt's lymphoma (BL), B-cell prolymphocytic leukemia, Splenic marginal zone lymphoma, Hairy cell leukemia, Splenic lymphoma/leukemia, unclassifiable, Splenic diffuse red pulp small B-cell lymphoma, Hairy cell leukemia variant, Waldenstrom macroglobulinemia, Heavy chain diseases, a Heavy chain disease, γ Heavy chain disease, Heavy chain disease, Plasma cell myeloma, Solitary plasmacytoma of bone, Extraosseous plasmacytoma, Extranodal marginal zone lymphoma of mucosa-associated lymphoid tissue (MALT lymphoma), Nodal marginal zone lymphoma, Pediatric nodal marginal zone lymphoma, Pediatric follicular lymphoma, Primary cutaneous follicle centre lymphoma, T-cell/histiocyte rich large B-cell lymphoma, Primary DLBCL of the CNS, Primary cutaneous DLBCL, leg type, EBV-positive DLBCL of the elderly, DLBCL associated with chronic inflammation, Lymphomatoid granulomatosis, Primary mediastinal (thymic) large B-cell lymphoma, Intravascular large B-cell lymphoma, ALK-positive large B-cell lymphoma, Plasmablastic lymphoma, Large B-cell lymphoma arising in HHV8-associated multicentric Castleman disease, Primary effusion lymphoma: B-cell lymphoma, unclassifiable, with features intermediate between diffuse large B-cell lymphoma and Burkitt lymphoma, and B-cell lymphoma, unclassifiable, with features intermediate between diffuse large B-cell lymphoma and classical Hodgkin lymphoma. In some embodiments, the preferred cancer is germinal-center B-cell-like (GCB) DLBCL, activated B-cell-like (ABC) DLBCL, follicular lymphoma (FL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), Waldenstrom macroglobulinemia (WM), central nervous system lymphoma (CNSL), or Burkitt's lymphoma (BL).

In some embodiments, the autoimmune disorder is selected from the group consisting of rheumatoid arthritis, juvenile rheumatoid arthritis, systemic lupus erythematosus (SLE), Wegener's disease, inflammatory bowel disease, idiopathic thrombocytopenic purpura (ITP), thrombotic thrombocytopenic purpura (TTP), autoimmune thrombocytopenia, multiple sclerosis, psoriasis, IgA nephropathy, IgM polyneuropathies, myasthenia gravis, vasculitis, diabetes mellitus, Reynaud's syndrome, Sjorgen's syndrome, glomerulonephritis, Neuromyelitis Optica (NMO), and IgG neuropathy.

In some embodiments, the bispecific antibody is in a kit comprising: (a) a composition comprising any one of the preceding bispecific antibodies and (b) a package insert comprising instructions for administering the composition to a subject to treat or delay progression of a cell proliferative disorder. In some embodiments, the kit is lyophilized.

In some embodiments, the method further comprises administering to a subject an antibody-drug conjugate (“ADC”) comprising a bispecific antibody set forth herein and a drug. In some embodiments, the method further comprises administering to the subject a glucocorticoid, rituximab, obinutuzumab, and/or an antibody-drug conjugate (ADC).

In any of the preceding uses or methods, the subject can be a human.

### BRIEF DESCRIPTION OF THE DRAWINGS

The application file contains at least one drawing executed in color. Copies of this patent or patent application with color drawings will be provided by the Office upon request and payment of the necessary fee.

FIG. 1 depicts a sequence alignment of heavy chain domains. A consensus sequence is set forth on the top line. The SP34 heavy chain sequence is set forth in the line numbered line 1. Lines 2, 3, and 4 set forth the VH3, VH4, and VH5, respectively, humanized heavy chains of the invention. CDRs are set forth in underline between the SP34 heavy chain and the VH3, VH4, and VH5 sequences. The inset at the bottom shows the chain name, germline, and a note with respect to the respective sequences.

FIG. 2 depicts a sequence alignment of light chain domains. A consensus sequence is set forth on the top line. The SP34 light chain sequence is set forth in the line numbered line 1. Lines 2, 3, and 4 set forth the VL4, VL5, and VL6, respectively, humanized light chains of the invention. CDRs are set forth in underline between the SP34 light chain and the VL4, VL5, and VL6 sequences. The inset at the bottom shows the chain name, germline, and a note with respect to the respective sequences.

FIG. 3 depicts a humanization summary for one embodiment of the invention. The left column sets forth the construct number. The next column sets forth individual components of the constructs. The next two columns set forth production and purification results, respectively. The last three columns set fort ELISA, FACS, and T-cell stimulation results, respectively, according to the invention.

FIG. 4 depicts CE-SDS electropherogram results for respective parent constructs of some embodiments of the invention, which shows protein purity, reduced and non-reduced, indicative of the protein.

FIG. 5 depicts ELISA results for parent constructs of some embodiments of the invention, with the y-axis showing OD450 and the x-axis showing log concentration in g/ml. The inset shows EC50s for respective constructs.

FIG. 6 depicts FACS assay results for constructs of some embodiments of the invention, with the y-axis showing median FL1-H of positives and the x-axis showing log concentration in g/ml. The inset shows EC50s for respective constructs.

FIG. 7 depicts percentage proliferating and CFSE MFI of total lymphocytes. The insets show % proliferating and total MFI on the y-axis with x-axis showing log concentration in g/ml.

FIG. 8 depicts the illustrations for controls, 1:1 ratio designs and 2:1 ratio designs according to some embodiments of the invention.

FIG. 9 depicts a construct according to one embodiment of the invention (PP11515).

FIG. 10 depicts a construct according to one embodiment of the invention (PP11519).

FIG. 11 depicts a construct according to one embodiment of the invention (PP11520).

FIG. 12 depicts a construct according to one embodiment of the invention (PP11731).

FIG. 13 depicts a construct according to one embodiment of the invention (PP11521).

FIG. 14 depicts a construct according to one embodiment of the invention (PP11523).

FIG. 15 depicts results for results for Octet binding experiments for antibodies against HER2 according to some embodiments of the invention. The inset shows a table setting forth loading sample ID, sample ID, KD, kon, kdis, FullX2 and Full R2.

FIG. 16A and FIG. 16B depicts result tables for donor 1 (2664) for a viability test for percentage propidium iodide (PI) positive cells in carboxyfluorescein succinimidyl ester (CFSE) positive populations.

### DETAILED DESCRIPTION OF EMBODIMENTS OF THE INVENTION

The present invention relates to humanized anti-cluster of differentiation 3 (“CD3”) bispecific antibodies and methods of using the same.

### I. DEFINITIONS

The term “about” as used herein refers to the usual error range for the respective value readily known to the skilled person in this technical field. Reference to “about” a value or parameter herein includes (and describes) embodiments that are directed to that value or parameter per se.

An “acceptor human framework” for the purposes herein is a framework comprising the amino acid sequence of a light chain variable domain (V_{L}) framework or a heavy chain variable domain (V_{H}) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the V_{L} acceptor human framework is identical in sequence to the V_{L} human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein.

An “affinity matured” antibody refers to an antibody with one or more alterations in one or more hypervariable regions, compared to a parent antibody, which does not possess such alterations, such alterations resulting in an improvement in the affinity of the antibody for antigen.

The terms “anti-CD3 antibody” and “an antibody that binds to CD3” refer to an antibody that is capable of binding CD3 with sufficient affinity such that the antibody is useful as a diagnostic and/or therapeutic agent in targeting CD3. In one embodiment, the extent of binding of an anti-CD3 antibody to an unrelated, non-CD3 protein is less than about 10% of the binding of the antibody to CD3 as measured, e.g., by a radioimmunoassay (RIA). In certain embodiments, an antibody that binds to CD3 has a dissociation constant (Kd) of ≤1 μM, ≤100 nM, ≤10 nM, ≤1 nM, ≤0.1 nM, ≤0.01 nM, or ≤0.001 nM (e.g. 10−8M or less, e.g. from 10−8M to 10−13M, e.g., from 10−9 M to 10−13 M). In certain embodiments, an anti-CD3 antibody binds to an epitope of CD3 that is conserved among CD3 from different species.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

By “binding domain” is meant a part of a compound or a molecule that specifically binds to a target epitope, antigen, ligand, or receptor. Binding domains include but are not limited to antibodies (e.g., monoclonal, polyclonal, recombinant, humanized, and chimeric antibodies), antibody fragments or portions thereof (e.g., Fab fragments, Fab′2, scFv antibodies, SMIP, domain antibodies, diabodies, minibodies, scFv-Fc, affibodies, nanobodies, and VH and/or VL domains of antibodies), receptors, ligands, aptamers, and other molecules having an identified binding partner.

A “chemotherapeutic agent” is a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaII (see, e.g., Nicolaou et al., Angew. Chem Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL@), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); combretastatin; folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®, Bristol-Myers Squibb Oncology, Princeton, N.J.), albumin-engineered nanoparticle formulation of paclitaxel (ABRAXANE™), and docetaxel (TAXOTERE®, Rhome-Poulene Rorer, Antony, France); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R) (e.g., erlotinib (Tarceva™)); and VEGF-A that reduce cell proliferation; vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g. celecoxib or etoricoxib), proteosome inhibitor (e.g. PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors; tyrosine kinase inhibitors; serine-threonine kinase inhibitors such as rapamycin (sirolimus, RAPAMUNE®); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin, and pharmaceutically acceptable salts, acids or derivatives of any of the above; as well as combinations of two or more of the above. Some embodiments are also drawn to any combinations of one or more of the above with any other compounds, such as any other therapeutic compounds.

Chemotherapeutic agents as defined herein include “anti-hormonal agents” or “endocrine therapeutics” which act to regulate, reduce, block, or inhibit the effects of hormones that can promote the growth of cancer. They may be hormones themselves, including, but not limited to: anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Raf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; Vinorelbine and Esperamicins (see U.S. Pat. No. 4,675,187), and pharmaceutically acceptable salts, acids or derivatives of any of the above; as well as combinations of two or more of the above.

The term “chimeric” antibody refers to an antibody in which a portion of the heavy and/or light chain is derived from a particular source or species, while the remainder of the heavy and/or light chain is derived from a different source or species.

The term “cluster of differentiation 3” or “CD3,” as used herein, refers to any native CD3 from any vertebrate source, including mammals such as primates (e.g. humans) and rodents (e.g., mice and rats), unless otherwise indicated, including, for example, CD3ε, CD3γ, CD3α, and CD3β chains. The term encompasses “full-length,” unprocessed CD3 (e.g., unprocessed or unmodified CD3ε or CD3γ), as well as any form of CD3 that results from processing in the cell. The term also encompasses naturally occurring variants of CD3, including, for example, splice variants or allelic variants. CD3 includes, for example, human CD3ε protein (NCBI RefSeq No. NP-000724), which is 207 amino acids in length, and human CD3γ protein (NCBI RefSeq No. NP-000064), which is 182 amino acids in length.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

It is understood that aspects and embodiments of the invention described herein include “comprising,” “consisting,” and “consisting essentially of” aspects and embodiments.

The term “cytotoxic agent” as used herein refers to a substance that inhibits or prevents a cellular function and/or causes cell death or destruction. Cytotoxic agents include, but are not limited to, radioactive isotopes (e.g., At211, I131, I125, Y90, Re186, Re188, Sm153, Bi212, P32, Pb212 and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

A “disorder” is any condition that would benefit from treatment including, but not limited to, chronic and acute disorders or diseases including those pathological conditions which predispose the mammal to the disorder in question.

The terms “cell proliferative disorder” and “proliferative disorder” refer to disorders that are associated with some degree of abnormal cell proliferation. In one embodiment, the cell proliferative disorder is cancer. In one embodiment, the cell proliferative disorder is a tumor.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia or lymphoid malignancies. More particular examples of such cancers include, but are not limited to, squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); hairy cell leukemia; chronic myeloblastic leukemia; and post-transplant lymphoproliferative disorder (PTLD), as well as abnormal vascular proliferation associated with phakomatoses, edema (such as that associated with brain tumors), Meigs' syndrome, brain, as well as head and neck cancer, and associated metastases. In certain embodiments, cancers that are amenable to treatment by the antibodies of the invention include breast cancer, colorectal cancer, rectal cancer, non-small cell lung cancer, glioblastoma, non-Hodgkins lymphoma (NHL), renal cell cancer, prostate cancer, liver cancer, pancreatic cancer, soft-tissue sarcoma, kaposi's sarcoma, carcinoid carcinoma, head and neck cancer, ovarian cancer, mesothelioma, and multiple myeloma. In some embodiments, the cancer is selected from: small cell lung cancer, glioblastoma, neuroblastomas, melanoma, breast carcinoma, gastric cancer, colorectal cancer (CRC), and hepatocellular carcinoma. Yet, in some embodiments, the cancer is selected from: non-small cell lung cancer, colorectal cancer, glioblastoma and breast carcinoma, including metastatic forms of those cancers. In other embodiments, the cancer is selected from a class of mature B-Cell cancers excluding Hodgkin's Lymphoma but including germinal-center B-cell-like (GCB) DLBCL, activated B-cell-like (ABC) DLBCL, follicular lymphoma (FL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), Waldenstrom macroglobulinemia (WM), central nervous system lymphoma (CNSL), Burkitt's lymphoma (BL), B-cell prolymphocytic leukemia, Splenic marginal zone lymphoma, Hairy cell leukemia, Splenic lymphoma/leukemia, unclassifiable, Splenic diffuse red pulp small B-cell lymphoma, Hairy cell leukemia variant, Waldenstrom macroglobulinemia, Heavy chain diseases, a Heavy chain disease, γ Heavy chain disease, Heavy chain disease, Plasma cell myeloma, Solitary plasmacytoma of bone, Extraosseous plasmacytoma, Extranodal marginal zone lymphoma of mucosa-associated lymphoid tissue (MALT lymphoma), Nodal marginal zone lymphoma, Pediatric nodal marginal zone lymphoma, Pediatric follicular lymphoma, Primary cutaneous follicle centre lymphoma, T-cell/histiocyte rich large B-cell lymphoma, Primary DLBCL of the CNS, Primary cutaneous DLBCL, leg type, EBV-positive DLBCL of the elderly, DLBCL associated with chronic inflammation, Lymphomatoid granulomatosis, Primary mediastinal (thymic) large B-cell lymphoma, Intravascular large B-cell lymphoma, ALK-positive large B-cell lymphoma, Plasmablastic lymphoma, Large B-cell lymphoma arising in HHV8-associated multicentric Castleman disease, Primary effusion lymphoma: B-cell lymphoma, unclassifiable, with features intermediate between diffuse large B-cell lymphoma and Burkitt lymphoma, and B-cell lymphoma, unclassifiable, with features intermediate between diffuse large B-cell lymphoma and classical Hodgkin lymphoma.

“Tumor,” as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder” and “tumor” are not mutually exclusive as referred to herein.

The term “tumor antigen,” as used herein, may be understood as those antigens that are presented on tumor cells. These antigens can be presented on the cell surface with an extracellular part, which is often combined with a transmembrane and cytoplasmic part of the molecule. These antigens can sometimes be presented only by tumor cells and never by the normal ones. Tumor antigens can be exclusively expressed on tumor cells or might represent a tumor specific mutation compared to normal cells. In this case, they are called tumor-specific antigens. More common are tumor antigens that are presented by tumor cells and normal cells, and they are called tumor-associated antigens. These tumor-associated antigens can be overexpressed compared to normal cells or are accessible for antibody binding in tumor cells due to the less compact structure of the tumor tissue compared to normal tissue.

“Effector functions” refer to those biological activities attributable to the Fc region of an antibody, which vary with the antibody isotype. Examples of antibody effector functions include: C1 q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of a compound, for example, an anti-CD3 antibody of the invention or a composition (e.g., pharmaceutical composition) thereof, is at least the minimum amount required to achieve the desired therapeutic or prophylactic result, such as a measurable improvement or prevention of a particular disorder (e.g., a cell proliferative disorder, e.g., cancer). An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region residues. The FR of a variable domain generally consists of four FR domains: FR1, FR2, FR3, and FR4. Accordingly, the HVR and FR sequences generally appear in the following sequence in V_{H} (or VL): FR1-H1(L)-FR2-H2(L2)-FR3-H3(L3)-FR4.

The terms “full-length antibody,” “intact antibody,” and “whole antibody” are used herein interchangeably to refer to an antibody having a structure substantially similar to a native antibody structure or having heavy chains that contain an Fc region as defined herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol. 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

A “human consensus framework” is a framework which represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the V_{L}, the subgroup is subgroup kappa I as in Kabat et al., supra. In one embodiment, for the V_{H}, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the V_{H} (H1, H2, H3), and three in the V_{L}(L1, L2, L3). Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

A “subject” or an “individual” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the subject or individual is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-CD3 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (V_{H}), also called a variable heavy domain or a heavy chain variable domain, followed by three constant domains (CH1, CH2, and CH3). Similarly, from N- to C-terminus, each light chain has a variable region (V_{L}), also called a variable light domain or a light chain variable domain, followed by a constant light (CL) domain. The light chain of an antibody may be assigned to one of two types, called kappa (K) and lambda (A), based on the amino acid sequence of its constant domain.

The term “package insert” is used to refer to instructions customarily included in commercial packages of therapeutic products, that contain information about the indications, usage, dosage, administration, combination therapy, contraindications, and/or warnings concerning the use of such therapeutic products.

The term “protein,” as used herein, refers to any native protein from any vertebrate source, including mammals such as primates (e.g. humans) and rodents (e.g., mice and rats), unless otherwise indicated. The term encompasses “full-length,” unprocessed protein as well as any form of the protein that results from processing in the cell. The term also encompasses naturally occurring variants of the protein, e.g., splice variants or allelic variants.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

As used herein, “delaying progression” of a disorder or disease means to defer, hinder, slow, retard, stabilize, and/or postpone development of the disease or disorder (e.g., a cell proliferative disorder, e.g., cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

By “reduce” or “inhibit” is meant the ability to cause an overall decrease, for example, of 20% or greater, of 50% or greater, or of 75%, 85%, 90%, 95%, or greater. In certain embodiments, reduce or inhibit can refer to the effector function of an antibody that is mediated by the antibody Fc region, such effector functions specifically including complement-dependent cytotoxicity (CDC), antibody-dependent cellular cytotoxicity (ADCC), and antibody-dependent cellular phagocytosis (ADCP).

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

As used herein, “administering” is meant a method of giving a dosage of a compound (e.g., an anti-CD3 antibody of the invention or a nucleic acid encoding an anti-CD3 antibody of the invention) or a composition (e.g., a pharmaceutical composition, e.g., a pharmaceutical composition including an anti-CD3 antibody of the invention) to a subject. The compositions utilized in the methods described herein can be administered, for example, intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. The method of administration can vary depending on various factors (e.g., the compound or composition being administered and the severity of the condition, disease, or disorder being treated).

### II. COMPOSITIONS AND METHODS

In one aspect, the invention is based, in part, on bispecific CD3 antibodies.

### A. Exemplary Anti-CD3 Antibodies

For example, some embodiments provide a bispecific antibody that binds to CD3 and another antigen, wherein the bispecific antibody comprises a first heavy chain binding domain and a second heavy chain binding domain, the first heavy chain binding domain comprising one or more of:
a. a V_{H} comprising: a CDR-H1 comprising a polypeptide comprising an amino acid sequence comprising SEQ ID NO: 5; a CDR-H2 comprising a polypeptide comprising an amino acid sequence comprising SEQ ID NO: 6; and a CDR-H3 comprising an amino acid sequence comprising a polypeptide comprising SEQ ID NO: 7;
   and the second heavy chain binding domain comprising one or more of:
b. a V_{H} comprising: a CDR-H1 comprising a polypeptide comprising an amino acid sequence comprising one or more of SEQ ID NOs: 20-21; a CDR-H2 comprising a polypeptide comprising an amino acid sequence comprising one or more of SEQ ID NOs: 22-23; and a CDR-H3 comprising a polypeptide comprising an amino acid sequence of one or more of SEQ ID NOs: 24-25.

Some embodiments provide a bispecific antibody that binds to CD3 and another antigen, wherein the bispecific antibody comprises a first light chain binding domain and a second light chain binding domain, the first light chain binding domain comprising one or more of:
a. a V_{L} comprising: a CDR-L1 comprising a polypeptide comprising an amino acid sequence of SEQ ID NO: 12; a CDR-L2 comprising a polypeptide comprising an amino acid sequence of SEQ ID NO: 13; and a CDR-L3 comprising a polypeptide comprising an amino acid sequence of SEQ ID NO: 14;
   and the second light chain binding domain comprising one or more of:
b. a V_{L} comprising: a CDR-L1 comprising a polypeptide comprising an amino acid sequence of SEQ ID NO: 28; a CDR-L2 comprising a polypeptide comprising an amino acid sequence of SEQ ID NO: 29; and a CDR-L3 comprising a polypeptide comprising an amino acid sequence of SEQ ID NO: 30.

Some embodiments provide a bispecific antibody, wherein the V_{H} comprises a polypeptide comprising an amino acid sequence of one or more of SEQ NOs: 1-4 and/or comprising an amino acid sequence of one or more of SEQ ID NOs: 15-19. Some embodiments provide a bispecific antibody, wherein the V_{L} comprises a polypeptide comprising an amino acid sequence of one or more of SEQ NOs: 8-11 and/or an comprising an amino acid sequence of SEQ ID NOs: 26-27. Some embodiments provide a bispecific antibody that binds to CD3 and another antigen, wherein the bispecific antibody comprises a first heavy chain binding domain comprising a polypeptide comprising an amino acid sequence of one of SEQ NOs: 1-4 and a second heavy chain binding domain comprising a polypeptide comprising an amino acid sequence of one of SEQ ID NOs: 15-19, first light chain binding domain comprising a polypeptide comprising an amino acid sequence of one of SEQ NOs: 8-11 and a second light chain binding domain comprising a polypeptide comprising an amino acid sequence of one of SEQ ID NOs: 26-27.

Some embodiments provide a bispecific antibody that binds to CD3 and another antigen, wherein the bispecific antibody comprises a first heavy chain binding domain comprising an a polypeptide comprising an amino acid sequence comprising at least about 90%, at least about 91%, at least about 92%, at least about 93%, at least about 94%, at least about 95%, at least about 96%, at least about 97%, at least about 98%, or at least about 99% sequence identity to one of SEQ NOs: 1-4 and a second heavy chain binding domain comprising a polypeptide comprising an amino acid sequence comprising at least about 90%, at least about 91%, at least about 92%, at least about 93%, at least about 94%, at least about 95%, at least about 96%, at least about 97%, at least about 98%, or at least about 99% sequence identity to one of SEQ NOs: 15-19, a first light chain binding domain comprising a polypeptide comprising an amino acid sequence comprising at least about 90%, at least about 91%, at least about 92%, at least about 93%, at least about 94%, at least about 95%, at least about 96%, at least about 97%, at least about 98%, or at least about 99% sequence identity to one of SEQ NOs: 8-11 and a second light chain binding domain comprising a polypeptide comprising an amino acid sequence comprising at least about 90%, at least about 91%, at least about 92%, at least about 93%, at least about 94%, at least about 95%, at least about 96%, at least about 97%, at least about 98%, or at least about 99% sequence identity to one of SEQ NOs: 26-27.

In some embodiments, the bispecific antibody comprises at least one (e.g., 1, 2, 3, or 4) of heavy chain framework regions FR-H1, FR-H2, FR-H3, and FR-H4 comprising a polypeptide comprising the sequences set forth herein, respectively, and/or at least one (e.g., 1, 2, 3, or 4) of the light chain framework regions FR-L1, FR-L2, FR-L3, and FR-L4 comprising the polypeptide comprising the sequences set forth herein, respectively.

In any of the above embodiments, the bispecific antibody is humanized. In one embodiment, the bispecific antibody comprises HVRs as in any of the above embodiments, and further comprises an acceptor human framework, e.g., a human immunoglobulin framework or a human consensus framework.

In another aspect, a bispecific antibody is provided, wherein the antibody comprises a V_{H} as in any of the embodiments provided above, and a V_{L} as in any of the embodiments provided above, wherein one or both of the variable domain sequences include post-translational modifications.

In a further aspect, the invention provides a bispecific antibody that binds to the same epitope as an anti-CD3 antibody provided herein. In some embodiments, the bispecific antibody binds a unique CD3 epitope. In some embodiments, the bispecific antibody makes unique contacts with amino acids of human CD3ε at a distance of 3.5 Angstroms, 3.25 Angstroms, 3.00 Angstroms, 2.75 Angstroms, or less. In some embodiments, the bispecific antibody binds to an epitope consisting of one, two, three, four, or five amino acids of human CD3ε at a distance of 3.5 Angstroms, 3.25 Angstroms, 3.00 Angstroms, 2.75 Angstroms or less. In one embodiment, the anti-CD3 antibody of the invention makes unique contacts with amino acids of human CD3ε at a distance of 3.5 Angstroms or less. In some embodiments, the bispecific antibody binds to an epitope consisting of one, two, three, four, or five amino acids of human CD3ε at a distance of 3.5 Angstroms or less.

An anti-CD3 epitope may be determined by anti-CD3 antibody binding to peptide fragments of the epitope. Alternatively, an anti-CD3 epitope may be determined by alanine scanning mutagenesis. In some embodiments, a reduction in binding of an anti-CD3 antibody to mutated CD3 by 20%, 30%, 50%, 80% or more indicates the amino acid residue of CD3 mutated in an alanine scanning mutagenesis assay is an epitope residue for that anti-CD3 antibody. Alternatively, an anti-CD3 epitope may be determined by mass spectrometry. In some embodiments, the epitope is determined by crystallography.

### 1. Antibody Affinity

In some embodiments, a bispecific antibody provided herein has a dissociation constant (Kd) of ≤1 μM, ≤100 nM, ≤10 nM, ≤1 nM, ≤0.1 nM, ≤0.01 nM, or ≤0.001 nM (e.g., 10−8M or less, e.g., from 10−8M to 10−13M, e.g., from 10−9M to 10−13 M).

In some embodiments, Kd is measured by a radiolabeled antigen binding assay (RIA). In some embodiments, an RIA is performed with the Fab version of an antibody of interest and its antigen. For example, solution binding affinity of Fabs for antigen is measured by equilibrating Fab with a minimal concentration of (125I)-labeled antigen in the presence of a titration series of unlabeled antigen, then capturing bound antigen with an anti-Fab antibody-coated plate (see, e.g., Chen et al., J. Mol. Biol. 293:865-881(1999)).

In some embodiments, Kd is measured using a BIACORE® surface plasmon resonance assay. For example, an assay using a BIACORE®-2000 or a BIACORE®-3000 (BIAcore, Inc., Piscataway, N.J.) is performed at 25° C. with immobilized antigen CM5 chips at ^{˜}10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 g/ml (^{˜}0.2 M) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kon) and dissociation rates (KO are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio kon/koff. See, for example, Chen et al., J. Mol. Biol. 293:865-881 (1999). If the on-rate exceeds 106 M−1s−1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### 2. Antibody Fragments

In some embodiments, an antibody provided herein is an antibody fragment. Antibody fragments include, but are not limited to, Fab, Fab′, Fab′-SH, F(ab′)2, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Diabodies are antibody fragments with two antigen-binding sites that may be bivalent or bispecific. See, e.g., EP 404,097; WO 1993/01161; Hudson et al. Nat. Med. 9:129-134 (2003); and Hollinger et al. Proc. Natl. Acad. Sci. USA 90: 6444-6448 (1993). Triabodies and tetrabodies are also described in Hudson et al. Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage).

### 3. Chimeric and Humanized Antibodies

In some embodiments, an antibody provided herein is a chimeric antibody. Certain chimeric antibodies are described, e.g., in U.S. Pat. No. 4,816,567; and Morrison et al. Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In some embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For a review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. LISA. 103; 3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain.

### 5. Library-Derived Antibodies

Antibodies of the invention may be isolated by screening combinatorial libraries for antibodies with the desired activity or activities. For example, a variety of methods is known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

### 6. Multispecific or Bispecific Antibodies

Multispecific antibodies are monoclonal antibodies that have binding specificities for at least two different sites. In some embodiments, bispecific antibodies may bind to two different epitopes of CD3 (e.g., CD3ε or CD3γ). In some embodiments, one of the binding specificities is for CD3 (e.g., CD3ε or CD3γ) and the other is for any other antigen (e.g., a second biological molecule, e.g., a cell surface antigen, e.g., a tumor antigen). Accordingly, a bispecific anti-CD3 antibody may have binding specificities for CD3 and a second biological molecule, such as a second biological molecule (e.g., a tumor antigen) listed in Table 1.

TABLE 1
Tumor antigen targets of the bispecific anti-CD3 antibodies of the invention

| | | | | | | |
|---|---|---|---|---|---|---|
| CD20 | CD79a | ETBR | IL13Ralpha2 | M-CSF | P2X5 | SSX-2 |
| 0772P | CD79b | ETV6- | IL20Rα | MCSP | p53 | SSX-4 |
| | | AML1 | | | | |
| | | fusion | | | | |
| | | protein | | | | |
| adipophilin | Cdc27 | EZH2 | Intestinal carboxyl | mdm-2 | PAP | STEAP1 |
| | | | esterase | | | |
| AIM-2 | CDK4 | FcRH1 | IRTA2 | MDP | PAX5 | STEAP1 |
| ALDH1A1 | CDKN2 | FcRH2 | Kallikrein 4 | ME1 | PBF | STEAP2 |
| | A | | | | | |
| alpha- | CEA | FcRH5 | KIF20A | Melan- | PMEL17 | survivin |
| actinin-4 | | | | A/ | | |
| | | | | MART- | | |
| | | | | 1 | | |
| alpha- | CLL1 | FLT3- | KK-LC-1 | Meloe | pml- | SYT-SSX1 or |
| foetoprotein | | ITD | | | | — |
| | | | | | RARalpha | SSX2 fusion |
| | | | | | fusion | protein |
| | | | | | protein | |
| Amphiregulin | CLPP | FN1 | KM-HN-1 | MMP-2 | PRAME | TAG-1 |
| ARTC1 | COA-1 | G250/ | K-ras | MMP-7 | PRDX5 | TAG-2 |
| | | MN/ | | | | |
| | | CAIX | | | | |
| ASLG659 | CPSF | GAGE- | LAGE-1 | MPF | PSCA | Telomerase |
| | | 1, 2, 8 | | | | |
| ASPHD1 | CRIPTO | GAGE- | LDLR- | MRP4 | PSCA hIg | TENB2 |
| | | 3, 4, 5, 6, | fucosyltransferaseASfus | | | |
| | | 7 | ion | | | |
| | | | protein | | | |
| B7-H4 | Cw6 | GDNF- | Lengsin | MSG78 | PSMA | TGF-betaRII |
| | | Ra1 | | 3 | | |
| BAFF-R | CXCR5 | GEDA | LGR5 | MUC1 | PTPRK | TMEFF1 |
| BAGE-1 | CXORF6 | GFRA1 | LY64 | MUC5 | RAB38/N | TMEM118 |
| | 1 | | | AC | Y- | |
| | | | | | MEL-1 | |
| BCLX (L) | cyclin D1 | glypican-3 | Ly6E | mucin | RAGE-1 | TMEM46 |
| BCR- | Cyclin- | GnTVf | Ly6G6D | MUM- | RBAF600 | TRAG-3 |
| ABL fusion | Al | | | 1f | | |
| protein | | | | | | |
| (b3a2) | | | | | | |
| beta- | dek- | gp100/ | LY6K | | MUM-2 RET | Triosephosphate |
| | can | | | | | |
| catenin | fusion | PmeI17 | | | | isomerase |
| | protein | | | | | |
| BING-4 | DKK1 | GPC3 | LYPD1 | MUM-3 | RGS5 | TRP-1/gp75 |
| B-RAF | DR1 | GPNMB | MAGE-A1 | Myosin | RhoC | TRP-2 |
| | | | | class I | | |
| Brevican | DR13 | GPR172 | MAGE-A10 | NA88- | RNF43 | TRP2-INT2 |
| | | A | | A | | |
| CALCA | E16 | GPR19 | MAGE-A12 | Napi2b | RNF43 | TrpM4 |
| CASP-5 | EDAR | GPR54 | MAGE-A2 | NCA | RU2AS | Tyrosinase |
| CASP-8 | EFTUD2 | HAVCR | MAGE-A3 | neo- | SAGE | tyrosinase |
| | | 1 | | PAP | | |
| CD19 | Elongation | HER2 | MAGE-A4 | NFYC | secernin 1 | VEGF |
| | factor 2 | | | | | |
| CD21 | ENAH | HER-2/ | MAGE-A6 | N-ras | Sema 5b | WT1 |
| | (hMe | neu | | | | |
| | na) | | | | | |
| CD22 | EpCAM | HERV- | MAGE-A9 | NY- | SIRT2 | XAGE- |
| | | K- | | BR-1 | | 1b/GAGED2a |
| | | MEL | | | | |
| CD33 | EphA3 | HLA- | MAGE-C1 | NY- | SLC35D3 | EGFR- |
| | | DOB | | ESO- | | T790M; |
| | | | | 1/LAG | | |
| | | | | E-2 | | |
| CD45 | EphB2R | hsp70-2 | MAGE-C2 | OAl | SNRPD1 | BMPR1B |
| CD70 | Epiregulin | IDO1 | mammaglobin-A | OGT | SOX10 | |
| CD72 | EGFR | IGF2B3 | MART2 | OS-9 | Sp17 | |
| EGFR- | EGFR- | EGFR- | EGFR-L858R | EGFR- | EGFR- | |
| G719A | G719C; | G719S; | | S768I | L861Q | |

The bispecific antibody (e.g., any one of the anti-CD3 antibodies described above) may have binding specificities for CD3 and a second biological molecule such as a human leukocyte antigen (HLA)-peptide complex presented on the cell surface by MHC. The bispecific antibody may have binding specificities for CD3 and a second biological molecule comprising a HLA-peptide complex selected from the group consisting of 0772P (CA125, MUC16; Genbank accession no. AF36148); adipophilin (perilipin-2, Adipose differentiation-related protein, ADRP, ADFP, MGC10598; NCBI Reference Sequence: NP 001113.2); AIM-2 (Absent In Melanoma 2, PYHIN4, Interferon-Inducible Protein AIM2; NCBI Reference Sequence: NP-004824.1); ALDH1 A1 (Aldehyde Dehydrogenase 1 Family, Member A1, ALDH1, PUMB1, Retinaldehyde Dehydrogenase 1, ALDC, ALDH-E1, ALHDII, RALDH 1, EC 1.2.1.36, ALDH11, HEL-9, HEL-S-53e, HEL12, RALDH1, Acetaldehyde Dehydrogenase 1, Aldehyde Dehydrogenase 1, Soluble, Aldehyde Dehydrogenase, Liver Cytosolic, ALDH Class 1, Epididymis Luminal Protein 12, Epididymis Luminal Protein 9, Epididymis Secretory Sperm Binding Protein Li 53e, Retinal Dehydrogenase 1, RaIDH1, Aldehyde Dehydrogenase Family 1 Member A1, Aldehyde Dehydrogenase, Cytosolic, EC 1.2.1; NCBI Reference Sequence: NP-000680.2); alpha-actinin-4 (ACTN4, Actinin, Alpha 4, FSGS1, Focal Segmental Glomerulosclerosis 1, Non-Muscle Alpha-Actinin 4, F-Actin Cross-Linking Protein, FSGS, ACTININ-4, Actinin Alpha4 Isoform, alpha-actinin-4; NCBI Reference Sequence: NP-004915.2); alpha-fetoprotein (AFP, HPAFP, FETA, alpha-1-fetoprotein, alpha-fetoglobulin, Alpha-1-fetoprotein, Alpha-fetoglobulin, HP; GenBank: AAB58754.1); Amphiregulin (AREG, SDGF, Schwannoma-Derived Growth Factor, Colorectum Cell-Derived Growth Factor, AR, CRDGF; GenBank: AAA51781.1); ARTC1 (ART1, ADP-Ribosyltransferase 1, Mono(ADP-Ribosyl)Transferase 1, ADP-Ribosyltransferase C2 And C3 Toxin-Like 1, ART2, CD296, RT6, ADP-Ribosyltransferase 2, GPI-Linked NAD(P)(+)-Arginine ADP-Ribosyltransferase 1, EC 2.4.2.31, CD296 Antigen; NP); ASLG659; ASPHDI (Aspartate Beta-Hydroxylase Domain Containing 1, Aspartate Beta-Hydroxylase Domain-Containing Protein 1, EC 1.14.11.-, EC 1.14.11; GenBank: AAI44153.1); B7-H4 (VTCN1, V-Set Domain Containing T Cell Activation Inhibitor 1, B7H4, B7 Superfamily Member 1, Immune Costimulatory Protein B7-H4, B7h.5, T-Cell Costimulatory Molecule B7x, B7S1, B7X, VCTN1, H4, B7 Family Member, PRO1291, B7 Family Member, H4, T Cell Costimulatory Molecule B7x, V-Set Domain-Containing T-Cell Activation Inhibitor 1, Protein B7S1; GenBank: AAZ17406.1); BAFF-R (TNFRSF13C, Tumor Necrosis Factor Receptor Superfamily, Member 13C, BAFFR, B-Cell-Activating Factor Receptor, BAFF Receptor, BLyS Receptor 3, CVID4, BROMIX, CD268, B Cell-Activating Factor Receptor, prolixin, Tumor Necrosis Factor Receptor Superfamily Member 13C, BR3, CD268 Antigen; NCBI Reference Sequence: NP-443177.1); BAGE-1; BCLX (L); BCR-ABL fusion protein (b3a2); beta-catenin (CTNNB1, Catenin (Cadherin-Associated Protein), Beta 1, 88 kDa, CTNNB, MRD19, Catenin (Cadherin-Associated Protein), Beta 1 (88 kD), armadillo, Catenin Beta-1; GenBank: CAA61107.1); BING-4 (WDR46, WD Repeat Domain 46, C6orf11, BING4, WD Repeat-Containing Protein BING4, Chromosome 6 Open Reading Frame 11, FP221, UTP7, WD Repeat-Containing Protein 46; NP); BMPR1 B (bone morphogenetic protein receptor-type IB, Genbank accession no. NM-00120; NP); B-RAF (Brevican (BCAN, BEHAB, Genbank accession no. AF22905); Brevican (BCAN, Chondroitin Sulfate Proteoglycan 7, Brain-Enriched Hyaluronan-Binding Protein, BEHAB, CSPG7, Brevican Proteoglycan, Brevican Core Protein, Chondroitin Sulfate Proteoglycan BEHAB; GenBank: AAH27971.1); CALCA (Calcitonin-Related Polypeptide Alpha, CALC1, Calcitonin 1, calcitonin, Alpha-Type CGRP, Calcitonin Gene-Related Peptide I, CGRP-I, CGRP, CGRP1, CT, KC, Calcitonin/Calcitonin-Related Polypeptide, Alpha, katacalcin; NP); CASP-5 (CASP5, Caspase 5, Apoptosis-Related Cysteine Peptidase, Caspase 5, Apoptosis-Related Cysteine Protease, Protease ICH-3, Protease TY, ICE(rel)-111, ICE(rel)III, ICEREL-III, ICH-3, caspase-5, TY Protease, EC 3.4.22.58, ICH3, EC 3.4.22; NP); CASP-8; CD19 (CD19-B-lymphocyte antigen CD19 isoform 2 precursor, B4, CVID3 [Homo sapiens], NCBI Reference Sequence: NP 001761.3); CD20 (CD20-B-lymphocyte antigen CD20, membrane-spanning 4-domains, subfamily A, member 1, B1, Bp35, CD20, CVID5, LEU-16, MS4A2, S7; NCBI Reference Sequence: NP-690605.1); CD21 (CD21 (CR2 (Complement receptor or C3DR (C3d/Epstein Barr virus receptor) or Hs.73792 Genbank accession no. M2600); (CD22 (B-cell receptor CD22-B isoform, BL-CAM, Lyb-8, LybB, SIGLEC-2, FLJ22814, Genbank accession No. AK02646); CD22; CD33 (CD33 Molecule, CD33 Antigen (Gp67), Sialic Acid Binding Ig-Like Lectin 3, Sialic Acid-Binding Ig-Like Lectin 3, SIGLEC3, gp67, SIGLEC-3, Myeloid Cell Surface Antigen CD33, p67, Siglec-3, CD33 Antigen; GenBank: AAH28152.1); CD45; CD70 (CD70-tumor necrosis factor (ligand) superfamily, member 7; surface antigen CD70; Ki-24 antigen; CD27 ligand; CD27-L; tumor necrosis factor ligand superfamily member 7; NCBI Reference Sequence for species Homo sapiens: NP 001243.1); CD72 (CD72 (B-cell differentiation antigen CD72, Lyb-; 359 aa, 1: 8.66, MW: 40225, TM: 1 [P] Gene Chromosome: 9p13.3, Genbank accession No. NP-001773.); CD79a (CD79a (CD79A, CD79a, immunoglobulin-associated alpha, a B cell-specific protein that covalently interacts with Ig beta (CD79B) and forms a complex on the surface with Ig M molecules, transduces a signal involved in B-cell differentiation), l: 4.84, MW: 25028 TM: 2 [P] Gene Chromosome: 19q13.2, Genbank accession No. NP-001774.1); CD79b (CD79b (CD79B, CD79b, IGb (immunoglobulin-associated beta), B29, Genbank accession no. NM 000626 or 1103867); Cdc27 (Cell Division Cycle 27, DOS1430E, D17S978E, Anaphase Promoting Complex Subunit 3, Anaphase-Promoting Complex Subunit 3, ANAPC3, APC3, CDCl27Hs, H-NUC, CDCl27 Homolog, Cell Division Cycle 27 Homolog (S. Cerevisiae), HNUC, NUC2, Anaphase-Promoting Complex, Protein 3, Cell Division Cycle 27 Homolog, Cell Division Cycle Protein 27 Homolog, Nuc2 Homolog; GenBank: AAH11656.1); CDK4 (Cyclin-Dependent Kinase 4, Cell Division Protein Kinase 4, PSK-J3, EC 2.7.11.22, CMM3, EC 2.7.11; NCBI Reference Sequence: NP-000066.1); CDKN2A (Cyclin-Dependent Kinase Inhibitor 2A, MLM, CDKN2, MTS1, Cyclin-Dependent Kinase Inhibitor 2A (Melanoma, P16, Inhibits CDK4), Cyclin-Dependent Kinase 4 Inhibitor A, Multiple Tumor Suppressor 1, CDK4I, MTS-1, CMM2, P16, ARF, INK4, INK4A, P14, P14ARF, P16-INK4A, P16INK4, P16INK4A, P19, P19ARF, TP16, CDK4 Inhibitor P16-INK4, Cell Cycle Negative Regulator Beta, p14ARF, p16-INK4, p16-INK4a, p16INK4A, p19ARF; NP); CEA; CLL1 (CLL-1 (CLEC12A, MICL, and DCAL, encodes a member of the C-type lectin/C-type lectin-like domain (CTL/CTLD) superfamily.

In some embodiments, bispecific antibodies may also be used to localize cytotoxic agents to cells which express a tumor antigen, such as a tumor antigen listed in Table 1 (e.g., CD20, FcRH5, HER2, LYPD1, LY6G6D, PMEL17, LY6E, CD19, CD33, CD22, CD79A, CD79B, EDAR, GFRA1, MRP4, RET, Steap1, or TenB2). Bispecific antibodies can also be prepared as full length antibodies or antibody fragments. In some embodiments, the antigen is Epcam, PSMA, BCMA, or ROR1.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). “Knob-in-hole” engineering of multispecific antibodies may be utilized to generate a first arm containing a knob and a second arm containing the hole into which the knob of the first arm may bind. The knob of the multispecific antibodies of the invention may be an anti-CD3 arm in one embodiment. Alternatively, the knob of the multispecific antibodies of the invention may be an anti-target/antigen arm in one embodiment. The hole of the multispecific antibodies of the invention may be an anti-CD3 arm in one embodiment. Alternatively, the hole of the multispecific antibodies of the invention may be an anti-target/antigen arm in one embodiment.

There other ways of making multispecific antibodies. For example, multispecific antibodies may be engineered using immunoglobulin crossover (also known as Fab domain exchange or CrossMab format) technology (see e.g., WO2009/080253; Schaefer et al., Proc. Natl. Acad. Sci. USA, 108:11187-11192 (2011)). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. ImmunoL, 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. ImmunoL, 152:5368 (1994)); and preparing trispecific antibodies as described, e.g., in Tutt et al. J. ImmunoL 147: 60 (1991).

### 7. Antibody Variants

In certain embodiments, amino acid sequence variants of the anti-CD3 antibodies of the invention (e.g., bispecific anti-CD3 antibodies of the invention that bind to CD3 and a second biological molecule, e.g., a cell surface antigen, e.g., a tumor antigen, such as TDB antibodies of the invention or variants thereof) are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

a. Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 2 under the heading of “preferred substitutions.” More substantial changes are provided in Table 2 under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved ADCC or CDC.

TABLE 2
Exemplary and Preferred Amino Acid
Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; Ala; | Ile |
| | Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

Amino acids may be grouped according to common side-chain properties:
(1) hydrophobic: Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.
Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant V_{H} or V_{L} being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant V_{H} and V_{L} sequences provided above, each HVR either is unaltered, or contains no more than one, two, or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen may be used. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

b. Glycosylation Variants

In certain embodiments, anti-CD3 antibodies of the invention can be altered to increase or decrease the extent to which the antibody is glycosylated. Addition or deletion of glycosylation sites to anti-CD3 antibody of the invention may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, anti-CD3 antibody variants are provided having a carbohydrate structure that lacks fucose attached (directly or indirectly) to an Fc region. For example, the amount of fucose in such antibody may be from 1% to 80%, from 1% to 65%, from 5% to 65%, or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about 3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lecl3 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Anti-CD3 antibodies variants are further provided with bisected oligosaccharides, for example, in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

c. Fc Region Variants

In certain embodiments, one or more amino acid modifications may be introduced into the Fc region of an anti-CD3 antibody of the invention thereby generating an Fc region variant (see e.g., US 2012/0251531). The Fc region variant may comprise a human Fc region sequence (e.g., a human IgG1, IgG2, IgG3 or IgG4 Fc region) comprising an amino acid modification (e.g., a substitution) at one or more amino acid positions. In certain embodiments, the invention contemplates an anti-CD3 antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96 non-radioactive cytotoxicity assay (Promega, Madison, Wis.). Useful effector cells for such assays include peripheral blood mononuclear cells (PBMC) and Natural Killer (NK) cells.

Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al. J. ImmunoL Methods 202:163 (1996); Cragg, M. S. et al. Blood. 101:1045-1052 (2003); and Cragg, M. S. and M. J. Glennie Blood. 103:2738-2743 (2004)). FcRn binding and in vivo clearance/half life determinations can also be performed using methods known in the art (see, e.g., Petkova, S. B. et al. Int'l. ImmunoL 18(12):1759-1769 (2006)).

Antibodies with reduced effector function include those with substitution of one or more of Fc region residues 238, 265, 269, 270, 297, 327 and 329 (U.S. Pat. Nos. 6,737,056 and 8,219,149). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

In certain embodiments, the proline at position 329 of a wild-type human Fc region in the antibody is substituted with glycine or arginine or an amino acid residue large enough to destroy the proline sandwich within the Fc/Fcγ receptor interface that is formed between the proline 329 of the Fc and tryptophan residues Trp 87 and Trp 110 of FcgRIII (Sondermann et al.: Nature 406, 267-273 (20 Jul. 2000)). In certain embodiments, the antibody comprises at least one further amino acid substitution. In one embodiment, the further amino acid substitution is S228P, E233P, L234A, L235A, L235E, N297A, N297D, or P331S, and still in another embodiment the at least one further amino acid substitution is L234A and L235A of the human IgG1 Fc region or S228P and L235E of the human IgG4 Fc region (see e.g., US 2012/0251531), and still in another embodiment the at least one further amino acid substitution is L234A and L235A and P329G of the human IgG1 Fc region.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821; and WO 94/29351 concerning other examples of Fc region variants.

In some aspects the bispecific antibody comprises an Fc region comprising an N297G mutation. In some embodiments, the bispecific antibody comprising the N297G mutation comprises one or more heavy chain constant domains, wherein the one or more heavy chain constant domains are selected from a first CH1 domain, a first CH2 domain, a first CH3 domain, a second CH1 domain, second CH2 domain, and a second CH3 domain.

d. Cysteine Engineered Antibody Variants

In certain embodiments, it may be desirable to create cysteine engineered antibodies in which one or more residues of an antibody are substituted with cysteine residues. In some embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, for example, in WO 2016/040856, which is incorporated by reference in its entirety herein, including any drawings.

e. Antibody Derivatives

In certain embodiments, the bispecific antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### B. Recombinant Methods and Compositions

The bispecific antibodies of the invention may be produced using recombinant methods and compositions, for example, as described in U.S. Pat. No. 4,816,567. In one embodiment, an isolated nucleic acid encoding an anti-CD3 antibody described herein is provided. Such nucleic acid may encode an amino acid sequence comprising the V_{L} and/or an amino acid sequence comprising the V_{H} of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the V_{L} of the antibody and an amino acid sequence comprising the V_{H} of the antibody, or (2) a first vector comprising a nucleic acid that encodes an amino acid sequence comprising the V_{L} of the antibody and a second vector comprising a nucleic acid that encodes an amino acid sequence comprising the V_{H} of the antibody. In one embodiment, the host cell is eukaryotic, e.g. a Chinese Hamster Ovary (CHO) cell or lymphoid cell (e.g., Y0, NS0, Sp20 cell). In one embodiment, a method of making a bispecific antibody is provided, wherein the method comprises culturing a host cell comprising a nucleic acid encoding the antibody under conditions suitable for expression of the antibody, and optionally recovering the antibody from the host cell (or host cell culture medium).

For recombinant production of a bispecific antibody, a nucleic acid encoding a bispecific antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### C. Assays

The bispecific antibodies of the invention may be identified, screened for, or characterized for their physical/chemical properties and/or biological activities by various assays known in the art.

### 1. Binding Assays and Other Assays

In one aspect, the bispecific antibody of the invention is tested for its antigen binding activity, for example, by known methods such as ELISA, Western blot, etc. In another aspect, competition assays may be used to identify an antibody that competes with an anti-CD3 antibody of the invention for binding to CD3. In an exemplary competition assay, immobilized CD3 is incubated in a solution comprising a first labeled antibody that binds to CD3 and a second unlabeled antibody that is being tested for its ability to compete with the first antibody for binding to CD3. The second antibody may be present in a hybridoma supernatant. As a control, immobilized CD3 is incubated in a solution comprising the first labeled antibody but not the second unlabeled antibody. After incubation under conditions permissive for binding of the first antibody to CD3, excess unbound antibody is removed, and the amount of label associated with immobilized CD3 is measured. If the amount of label associated with immobilized CD3 is substantially reduced in the test sample relative to the control sample, then that indicates that the second antibody is competing with the first antibody for binding to CD3. See, e.g., Harlow and Lane (1988) Antibodies: A Laboratory Manual. Ch. 14 (Cold Spring Harbor Laboratory, Cold Spring Harbor, N.Y.).

### 2. Activity Assays

In one aspect, assays are provided for identifying bispecific antibodies having biological activity. Biological activity may include, for example, binding to CD3 (e.g., CD3 on the surface of a T cell), or a peptide fragment thereof, either in vivo, in vitro, or ex vivo. In the case of a bispecific antibody of the invention, biological activity may also include, for example, effector cell activation (e.g., T cell (e.g., CD8+ and/or CD4+ T cell) activation), effector cell population expansion (i.e., an increase in T cell count), target cell population reduction (i.e., a decrease in the population of cells expressing the second biological molecule on their cell surfaces), and/or target cell killing. In some embodiments, the activity comprises ability to support B cell killing and/or the activation of the cytotoxic T cells.

### D. Immunoconjugates

The invention also provides immunoconjugates comprising a bispecific antibody herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065. In some embodiments, an immunoconjugate comprises a bispecific antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, sapaonaria officinalis inhibitor, gelonin, mitogellin, restrictocin, phenomycin, enomycin, and the tricothecenes.

In another embodiment, an immunoconjugate comprises a bispecific antibody as described herein conjugated to a radioactive atom to form a radioconjugate. A variety of radioactive isotopes are available for the production of radioconjugates. Examples include At211, I131, I125, Y90, Re186, Re188, Sm153, Bi212, P32, Pb212 and radioactive isotopes of Lu. When the radioconjugate is used for detection, it may comprise a radioactive atom for scintigraphic studies, for example tc99m or 1123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to, such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

### E. Methods and Compositions for Diagnostics and Detection

In certain embodiments, any of the bispecific antibodies of the invention may be used to detect the presence of CD3 in a biological sample. The term “detecting” as used herein encompasses quantitative or qualitative detection. In certain embodiments, a biological sample comprises a cell or tissue. In certain embodiments, the method comprises contacting the biological sample with an anti-CD3 antibody as described herein under conditions permissive for binding of the bispecific antibody to CD3 and another antigen, and detecting whether a complex is formed between the bispecific antibody and CD3. Such method may be an in vitro or in vivo method.

In certain embodiments, labeled bispecific antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes 32P, 14C, 125I, 3H, and 131I, fluorophores such as rare earth chelates or fluorescein and its derivatives, rhodamine and its derivatives, dansyl, umbelliferone, luceriferases, e.g., firefly luciferase, and bacterial luciferase (see for example, U.S. Pat. No. 4,737,456, which is incorporated by reference in its entirety herein, including any drawings), luciferin, 2,3-dihydrophthalazinediones, horseradish peroxidase (HRP), alkaline phosphatase, 0-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### F. Pharmaceutical Formulations

Pharmaceutical formulations of the bispecific antibody of the invention may be prepared by mixing such antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other. For example, it may be desirable to further provide an additional therapeutic agent (e.g., a chemotherapeutic agent, a cytotoxic agent, a growth inhibitory agent, and/or an anti-hormonal agent, such as those recited herein above). Such active ingredients are suitably present in combination in amounts that are effective for the purpose intended. Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the bispecific antibody, which matrices are in the form of shaped articles, for example, films, or microcapsules. The formulations to be used for in vivo administration are generally sterile. Sterility may be readily accomplished, e.g., by filtration through sterile filtration membranes.

### G. Therapeutic Methods and Compositions

Any of the bispecific antibodies of the invention may be used in therapeutic methods. In one embodiment, a bispecific antibody for use as a medicament is provided. In some embodiments, a bispecific antibody for use in treating or delaying progression of a cell proliferative disorder (e.g., cancer) or an autoimmune disorder (e.g., arthritis) is provided. In some embodiments, a bispecific antibody for use in a method of treatment is provided. In some embodiments, the invention provides a bispecific antibody for use in a method of treating an individual having a cell proliferative disorder or an autoimmune disorder comprising administering to the individual an effective amount of the bispecific antibody. In some embodiments, the method further comprises administering to the individual an effective amount of at least one additional therapeutic agent. In some embodiments, the invention provides a bispecific antibody for use in enhancing immune function in an individual having a cell proliferative disorder or an autoimmune disorder. In some embodiments, the invention provides a bispecific antibody for use in a method of enhancing immune function in an individual having a cell proliferative disorder or an autoimmune disorder comprising administering to the individual an effective of the bispecific antibody to activate effector cells (e.g., T cells, e.g., CD8+ and/or CD4+ T cells), expand (increase) an effector cell population, reduce a target cell population, and/or kill a target cell (e.g., target tumor cell). An “individual” according to any of the above embodiments may be a human.

In some embodiments, the invention provides for the use of a bispecific antibody in the manufacture or preparation of a medicament. In some embodiments, the medicament is for treatment of a cell proliferative disorder (e.g., cancer) or an autoimmune disorder (e.g., arthritis). In some embodiments, the medicament is for use in a method of treating a cell proliferative disorder or an autoimmune disorder comprising administering to an individual having a cell proliferative disorder or an autoimmune disorder an effective amount of the medicament. In some embodiments, the method further comprises administering to the individual an effective amount of at least one additional therapeutic agent. In some embodiments, the medicament is for activating effector cells (e.g., T cells, e.g., CD8+ and/or CD4+ T cells), expanding (increasing) an effector cell population, reducing a target cell population, and/or killing target cells (e.g., target tumor cells) in the individual. In some embodiments, the medicament is for use in a method of enhancing immune function in an individual having a cell proliferative disorder or an autoimmune disorder comprising administering to the individual an effective amount of the medicament to activate effector cells (e.g., T cells, e.g., CD8+ and/or CD4+ T cells), expand (increase) an effector cell population, reduce a target cell population, and/or kill a target cell (e.g., target tumor cell).

Some embodiments provide a method for treating a cell proliferative disorder (e.g., cancer) or an autoimmune disorder (e.g., arthritis). In some embodiments, the method comprises administering to an individual having such a cell proliferative disorder or an autoimmune disorder an effective amount of a bispecific antibody according to the invention. In some embodiments, the method further comprises administering to the individual an effective amount of at least one additional therapeutic agent, for example, as described below.

In some embodiments, the invention provides a method for enhancing immune function in an individual having a cell proliferative disorder or an autoimmune disorder in an individual having a cell proliferative disorder or an autoimmune disorder. In some embodiments, the method comprises administering to the individual an effective amount of a bispecific antibody to activate effector cells (e.g., T cells, e.g., CD8+ and/or CD4+ T cells), expand (increase) an effector cell population, reduce a target cell population, and/or kill a target cell (e.g., target tumor cell).

In some embodiments, the invention provides a method for treating a hematological cancer, such as a B cell cancer (for example, mature B-cell lymphoma) by administering an effective amount of a bispecific antibody of the invention. In some embodiments, the mature B-cell lymphoma is a Non-Hodgkin's Lymphoma (NHL). In some embodiments, the NHL is selected from the group comprising: germinal-center B-cell-like (GCB) DLBCL, activated B-cell-like (ABC) DLBCL, follicular lymphoma (FL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), Waldenstrom macroglobulinemia (WM), central nervous system lymphoma (CNSL), Burkitt's lymphoma (BL), B-cell prolymphocytic leukemia, Splenic marginal zone lymphoma, Hairy cell leukemia, Splenic lymphoma/leukemia, unclassifiable, Splenic diffuse red pulp small B-cell lymphoma, Hairy cell leukemia variant, Waldenstrom macroglobulinemia, Heavy chain diseases, a Heavy chain disease, γ Heavy chain disease, Heavy chain disease, Plasma cell myeloma, Solitary plasmacytoma of bone, Extraosseous plasmacytoma, Extranodal marginal zone lymphoma of mucosa-associated lymphoid tissue (MALT lymphoma), Nodal marginal zone lymphoma, Pediatric nodal marginal zone lymphoma, Pediatric follicular lymphoma, Primary cutaneous follicle centre lymphoma, T-cell/histiocyte rich large B-cell lymphoma, Primary DLBCL of the CNS, Primary cutaneous DLBCL, leg type, EBV-positive DLBCL of the elderly, DLBCL associated with chronic inflammation, Lymphomatoid granulomatosis, Primary mediastinal (thymic) large B-cell lymphoma, Intravascular large B-cell lymphoma, ALK-positive large B-cell lymphoma, Plasmablastic lymphoma, Large B-cell lymphoma arising in HHV8-associated multicentric Castleman disease, Primary effusion lymphoma: B-cell lymphoma, unclassifiable, with features intermediate between diffuse large B-cell lymphoma and Burkitt lymphoma, and B-cell lymphoma, unclassifiable, with features intermediate between diffuse large B-cell lymphoma and classical Hodgkin lymphoma. In a preferred embodiment of the invention, the method comprises treating a cancer comprising germinal-center B-cell-like (GCB) DLBCL, activated B-cell-like (ABC) DLBCL, follicular lymphoma (FL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), Waldenstrom macroglobulinemia (WM), central nervous system lymphoma (CNSL), or Burkitt's lymphoma (BL).

In some embodiments, the additional therapy comprises an alkylating agent. In some embodiments, the alkylating agent is 4-[5-[Bis(2-chloroethyl)amino]-1-methylbenzimidazol-2-yl]butanoic acid and salts thereof. In some embodiments, the alkylating agent is bendamustine.

In some embodiments, the additional therapy comprises a BCL-2 inhibitor. In some embodiments, the BCL-2 inhibitor is 4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl} sulfonyl)-2-(1H-pyrrolo[2,3-b]pyridin-5-yloxy)benzamide and salts thereof. In some embodiments, the BCL-2 inhibitor is venetoclax (CAS #: 1257044-40-8).

In some embodiments, the invention provides a method wherein the additional therapeutic agent is a glucocorticoid. In some embodiments, the glucocorticoid is dexamethasone.

Combination therapies encompass combined administration (where two or more therapeutic agents are included in the same or separate formulations), and separate administration, in which case, administration of the bispecific antibody of the invention can occur prior to, simultaneously, and/or following, administration of the additional therapeutic agent or agents. In some embodiments, administration of the bispecific antibody and administration of an additional therapeutic agent occur within about one month, or within about one, two, or three weeks, or within about one, two, three, four, five, or six days, of each other. Bispecific antibodies of the invention can also be used in combination with radiation therapy.

Bispecific antibodies of the invention (and/or any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. In some embodiments, the antibody is administered by subcutaneous administration. In some embodiments, a bispecific antibody administered by subcutaneous injection exhibits a less toxic response in a patient than the same bispecific antibody administered by intravenous injection. Dosing can be by any suitable route, for example, by injections, such as intravenous or subcutaneous injections, depending in part on whether the administration is brief or chronic. Various dosing schedules including but not limited to single or multiple administrations over various time-points, bolus administration, and pulse infusion are contemplated herein.

Bispecific antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but may be optionally formulated, with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of a bispecific antibody of the invention (when used alone or in combination with one or more other additional therapeutic agents) will depend on the type of disease to be treated, the type of bispecific antibody, the severity and course of the disease, whether the bispecific antibody is administered for preventive or therapeutic purposes, previous therapy, the patient's clinical history and response to the bispecific antibody, and the discretion of the attending physician. The bispecific antibody is suitably administered to the patient at one time or over a series of treatments.

As a general proposition, the therapeutically effective amount of the bispecific antibody administered to human will be in the range of about 0.01 to about 100 mg/kg of patient body weight whether by one or more administrations. In some embodiments, the bispecific antibody used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In some embodiments, a bispecific antibody described herein is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles.

The dose may be administered as a single dose or as multiple doses (e.g., 2 or 3 doses), such as infusions. For repeated administrations over several days or longer, depending on the condition, the treatment would generally be sustained until a desired suppression of disease symptoms occurs. One exemplary dosage of the bispecific antibody would be in the range from about 0.05 mg/kg to about 10 mg/kg. Thus, one or more doses of about 0.5 mg/kg, 2.0 mg/kg, 4.0 mg/kg, or 10 mg/kg (or any combination thereof) may be administered to the patient. Such doses may be administered intermittently, for example, every week or every three weeks (e.g., such that the patient receives from about two to about twenty, or, for example, about six doses of the bispecific antibody). An initial higher loading dose, followed by one or more lower doses may be administered. The progress of this therapy is easily monitored by conventional techniques and assays.

In some embodiments, the methods may further comprise an additional therapy. The additional therapy may be radiation therapy, surgery, chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy. In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy may be a separate administration of one or more of the therapeutic agents described above.

### H. Articles of Manufacture

In some embodiments, an article of manufacture containing materials useful for the treatment, prevention and/or diagnosis of the disorders set forth herein is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a bispecific antibody according to the invention.

The label or package insert indicates that the composition is used for treating the condition of choice. Moreover, the article of manufacture may comprise (a) a first container with a composition contained therein, wherein the composition comprises a bispecific antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFJ), phosphate-buffered saline, Ringer's solution and dextrose solution. It may further include other materials desirable from a commercial and user standpoint, including other buffers, diluents, filters, needles, and syringes.

TABLE S
Sequences

| SEQ ID | | Region and/or | |
|---|---|---|---|
| NO | Molecule | Designation | Sequence |
| 1. | SP34VH | Heavy Chain | VQLVESGGGLVQPKGSLKLSCAASGFTFNTY |
| | | | AMNWVRQAPGKGLEWVARIRSKYNNYATYYA |
| | | | DSVKDRFTISRDDSQSILYLQMNNLKTEDTA |
| | | | MYYCVRHGNFGNSYVSWFAYWGQGTLVTVSA |
| 2. | VH3 | CD3 Heavy Chain | EVQLVESGGGLVQPGGSLRLSCAASGFTFNT |
| | | | YAMNWVRQAPGKGLEWVGRIRSKYNNYATYY |
| | | | AASVKGRFTVSRDDSKSSLYLQMNSLKTEDT |
| | | | AVYYCVRHGNFGNSYVSWFAYWGQGTMVTVS |
| | | | S |
| 3. | VH4 | CD3 Heavy Chain | EVQLVESGGGLVQPGGSLRLSCAASGFTFNT |
| | | | YAMNWVRQAPGKGLEWVARIRSKYNNYATYY |
| | | | ADSVKDRFTVSRDDSKNTAYLQMNSLKTEDT |
| | | | AVYYCVRHGNFGNSYVSWFAYWGQGTMVTVS |
| | | | S |
| 4. | VH5 | CD3 Heavy Chain | EVQLVESGGGLVQPGGSLRLSCAASGFTFNT |
| | | | YAMNWVRQAPGKGLEWVARIRSKYNNYATYY |
| | | | ADSVKGRFTVSRDDSKNTAYLQMNSLKTEDT |
| | | | AVYYCVRHGNFGNSYVSWFAYWGQGTMVTVS |
| | | | S |
| 5. | CDRH1 | CD3 CDRH1 | GFTFNTYA |
| 6. | CDRH2 | CD3 CDRH2 | IRSKYNNYAT |
| 7. | CDRH3 | CD3 CDRH3 | VRHGNFGNSYVSWFAY |
| 8. | SP34VL | Light Chain | QAVVTQESALTTSPGETVTLTCRSSTGAVTT |
| | | | SNYANWVQEKPDHLFTGLIGGTNFRAPGVPA |
| | | | RFSGSLIGDKAALTITGAQTEDEAIYFCALW |
| | | | YSNLWVFGGGTKLTVL |
| 9. | VL4 | CD3 Light Chain | QTVVTQEPSLTVSPGGTVTLTCGSSTGAVTT |
| | | | SNYPNWVQQKPGQAPRGLIGGTNFLAPGTPA |
| | | | RFSGSLLGGKAALTLSGVQPEDEAEYYCALW |
| | | | YSNLWVFGGGTKLTVL |
| 10. | VL5 | CD3 Light Chain | QTVVTQEPSLTVSPGGTVTLTCASSTGAVTT |
| | | | SNYANWVQQNPGQAPRGLIGGINKKAPGTPA |
| | | | RFSGSLLGGKAALTLSGVQPEDEAEYYCALW |
| | | | YSNLWVFGGGTKLTVL |
| 11. | VL6 | CD3 Light Chain | QTVVTQEPSLTVSPGGTVTLTCGSSTGA |
| | | | VTTSNYPNWVQQKPGQAPRGLIGGTNKK |
| | | | APGTPARFSGSLLGGKAALTLSGAQPED |
| | | | EAEYYCALWYSNLWVFGGGTKLTVL |
| 12. | CDRL1 | CD3 CDRL1 | TGAVTTSNY |
| 13. | CDRL2 | CD3 CDRL2 | GTN |
| 14. | CDRL3 | CD3 CDRL3 | ALWYSNLWV |
| 15. | Her 2 VH | Her 2 Heavy Chain | EVQLVESGGGLVQPGGSLRLSCAASGFN |
| | | | IKDTYIHWVRQAPGKGLEWVARIYPTNG |
| | | | YTRYADSVKGRFTISADTSKNTAYLQMN |
| | | | SLRAEDTAVYYCSRWGGDGFYAMDYWGQ |
| | | | GTLVTVSS |
| 16. | Her 2 VH | Her 2 Heavy Chain | EVQLVESGGGLVQPGGSLRLSCAASGFN |
| | | | IKDTYIHWVRQAPGKGLEWVARIYPTNG |
| | | | YTRYADSVKGRFTISADTSKNTAYLQMN |
| | | | SLRAEDTAVYYCSRWGGDGFYAMDYWGQ |
| | | | GTLVTVSSASTKGPSVFPLAPSSKSTSG |
| | | | GTAALGCLVKDYFPEPVTVSWNSGALTS |
| | | | GVHTFPAVLQSSGLYSLSSVVTVPSSSL |
| | | | GTQTYICNVNHKPSNTKVDKKVEPKSCD |
| | | | KTHTCPPCPAPELLGGPSVFLFPPKPKD |
| | | | TLMISRTPEVTCVVVDVSHEDPEVKFNW |
| | | | YVDGVEVHNAKTKPREEQYASTYRVVSV |
| | | | LTVLHQDWLNGKEYKCKVSNKALPAPIE |
| | | | KTISKAKGQPREPQVYTLPPSRDELTKN |
| | | | QVSLYCLVKGFYPSDIAVEWESNGQPEN |
| | | | NYKTTPPVLDSDGSFFLYSKLTVDKSRW |
| | | | QQGNVFSCSVMHEALHNHYTQKSLSLSP |
| | | | G |
| 17. | Her 2 VH | Her 2 Heavy Chain | EVQLVESGGGLVQPGGSLRLSCAASGFT |
| | | | FNTYAMNWVRQAPGKGLEWVARIRSKYN |
| | | | NYATYYADSVKDRFTVSRDDSKNTAYLQ |
| | | | MNSLKTEDTAVYYCVRHGNFGNSYVSWF |
| | | | AYWGQGTMVTVSS |
| 18. | Her 2 VH | Her 2 Heavy Chain | EVQLVESGGGLVQPGGSLRLSCAASGFN |
| | | | IKDTYIHWVRQAPGKGLEWVARIYPTNG |
| | | | YTRYADSVKGRFTISADTSKNTAYLQMN |
| | | | SLRAEDTAVYYCSRWGGDGFYAMDYWGQ |
| | | | GTLVTVSS |
| 19. | Her 2 VH | Her 2 Heavy Chain | EVQLVESGGGLVQPGGSLRLSCAASGFT |
| | | | FNTYAMNWVRQAPGKGLEWVARIRSKYN |
| | | | NYATYYADSVKGRFTVSRDDSKNTAYLQ |
| | | | MNSLKTEDTAVYYCVRHGNFGNSYVSWF |
| | | | AYWGQGTMVTVSS |
| 20. | CDRH1 | H2 CDRH1 | IKDTYI |
| 21. | CDRH1 | H2 CDRH1 | FNTYAM |
| 22. | CDRH2 | H2 CDRH2 | RIYPTNGYTR |
| 23. | CDRH2 | H2 CDRH2 | RIRSKYNNYA |
| 24. | CDRH3 | H3 CDRH3 | WGGDGFYAM |
| 25. | CDRH3 | H3 CDRH3 | HGNFGNSYVSWF |
| 26. | Her 2 VL | Her 2 Light Chain (4D5) | DIQMTQSPSSLSASVGDRVTITCRASQD |
| | | | VNTAVAWYQQKPGKAPKLLIYSASFLYS |
| | | | GVPSRFSGSRSGTDFTLTISSLQPEDFA |
| | | | TYYCQQHYTTPPTFGQGTKVEIK |
| 27. | Her 2 VL | Her 2 Light Chain | DIQMTQSPSSLSASVGDRVTITCRASQD |
| | | | VNTAVAWYQQKPGKAPKLLIYSASFLYS |
| | | | GVPSRFSGSRSGTDFTLTISSLQPEDFA |
| | | | TYYCQQHYTTPPTFGQGTKVEIKRTVAA |
| | | | PSVFIFPPSDEQLKSGTASVVCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 28. | CDRL1 | H2 CDRL1 | SQDVNTAVA |
| 29. | CDRL2 | H2 CDRL2 | SFL |
| 30. | CDRL3 | H2 CDRL3 | HYTTPP |
| 31. | scFv | LVH(anti-Her2)-(G4S)3- | EVQLVESGGGLVQPGGSLRLSCAASGFN |
| | | VL(anti-Her2)-SGGGGS- | IKDTYIHWVRQAPGKGLEWVARIYPTNG |
| | | VH(SP34 HC4)- | YTRYADSVKGRFTISADTSKNTAYLQMN |
| | | VEGGSGGSGGSGGSGGV- | SLRAEDTAVYYCSRWGGDGFYAMDYWGQ |
| | | VL(SP34 LC5)-HHHHHH | GTLVTVSS |
| | | | GGGGSGGGGSGGGGSDIQMTQSPSSLSA |
| | | | SVGDRVTITCRASQDVNTAVAWYQQKPG |
| | | | KAPKLLIYSASFLYSGVPSRFSGSRSGT |
| | | | DFTLTISSLQPEDFATYYCQQHYTTPPT |
| | | | FGQGTKVEIKSGGGGSEVQLVESGGGLV |
| | | | QPGGSLRLSCAASGFTFNTYAMNWVRQA |
| | | | PGKGLEWVARIRSKYNNYATYYADSVKD |
| | | | RFTVSRDDSKNTAYLQMNSLKTEDTAVY |
| | | | YCVRHGNFGNSYVSWFAYWGQGTMVTVS |
| | | | SVEGGSGGSGGSGGSGGVQTVVTQEPSL |
| | | | TVSPGGTVTLTCASSTGAVTTSNYANWV |
| | | | QQNPGQAPRGLIGGTNKKAPGTPARFSG |
| | | | SLLGGKAALTLSGVQPEDEAEYYCALWY |
| | | | SNLWVFGGGTKLTVLHHHHHH |
| 32. | Anti-Her2 | | DIQMTQSPSSLSASVGDRVTITCRASQD |
| | LC | | VNTAVAWYQQKPGKAPKLLIYSASFLYS |
| | | | GVPSRFSGSRSGTDFTLTISSLQPEDFA |
| | | | TYYCQQHYTTPPTFGQGTKVEIKRTVAA |
| | | | PSVFIFPPSDEQLKSGTASVVCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 33. | Anti-Her2 | Anti-Her2 heavy chain | EVQLVESGGGLVQPGGSLRLSCAASGFN |
| | HC | Knob, T366Y | IKDTYIHWVRQAPGKGLEWVARIYPTNG |
| | | | YTRYADSVKGRFTISADTSKNTAYLQMN |
| | | | SLRAEDTAVYYCSRWGGDGFYAMDYWGQ |
| | | | GTLVTVSSASTKGPSVFPLAPSSKSTSG |
| | | | GTAALGCLVKDYFPEPVTVSWNSGALTS |
| | | | GVHTFPAVLQSSGLYSLSSVVTVPSSSL |
| | | | GTQTYICNVNHKPSNTKVDKKVEPKSCD |
| | | | KTHTCPPCPAPELLGGPSVFLFPPKPKD |
| | | | TLMISRTPEVTCVVVDVSHEDPEVKFNW |
| | | | YVDGVEVHNAKTKPREEQYASTYRVVSV |
| | | | LTVLHQDWLNGKEYKCKVSNKALPAPIE |
| | | | KTISKAKGQPREPQVYTLPPSRDELTKN |
| | | | QVSLYCLVKGFYPSDIAVEWESNGQPEN |
| | | | NYKTTPPVLDSDGSFFLYSKLTVDKSRW |
| | | | QQGNVFSCSVMHEALHNHYTQKSLSLSP |
| | | | G |
| 34. | Anti-CD3 | SP34 scFv(HC4 + LC5) | EVQLVESGGGLVQPGGSLRLSCAASGFT |
| | HC | G4AG4 Fc Hole, Y407T | FNTYAMNWVRQAPGKGLEWVARIRSKYN |
| | | | NYATYYADSVKDRFTVSRDDSKNTAYLQ |
| | | | MNSLKTEDTAVYYCVRHGNFGNSYVSWF |
| | | | AYWGQGTMVTVSSGGGGSGGGGSGGGGS |
| | | | QTVVTQEPSLTVSPGGTVTLTCASSTGA |
| | | | VTTSNYANWVQQNPGQAPRGLIGGTNKK |
| | | | APGTPARFSGSLLGGKAALTLSGVQPED |
| | | | EAEYYCALWYSNLWVFGGGTKLTVLGGG |
| | | | GAGGGGDKTHTCPPCPAPELLGGPSVFL |
| | | | FPPKPKDTLMISRTPEVTCVVVDVSHED |
| | | | PEVKFNWYVDGVEVHNAKTKPREEQYAS |
| | | | TYRVVSVLTVLHQDWLNGKEYKCKVSNK |
| | | | ALPAPIEKTISKAKGQPREPQVYTLPPS |
| | | | RDELTKNQVSLTCLVKGFYPSDIAVEWE |
| | | | SNGQPENNYKTTPPVLDSDGSFFLTSKL |
| | | | TVDKSRWQQGNVFSCSVMHEALHNHYTQ |
| | | | KSLSLSPG |
| 35. | HC | SP34 scFv(HC4 + LC5) SG4S | EVQLVESGGGLVQPGGSLRLSCAASGFT |
| | | anti-Her2 heavy chain | FNTYAMNWVRQAPGKGLEWVARIRSKYN |
| | | Knob, T366Y | NYATYYADSVKDRFTVSRDDSKNTAYLQ |
| | | | MNSLKTEDTAVYYCVRHGNFGNSYVSWF |
| | | | AYWGQGTMVTVSSGGGGSGGGGSGGGGS |
| | | | QTVVTQEPSLTVSPGGTVTLTCASSTGA |
| | | | VTTSNYANWVQQNPGQAPRGLIGGTNKK |
| | | | APGTPARFSGSLLGGKAALTLSGVQPED |
| | | | EAEYYCALWYSNLWVFGGGTKLTVLSGG |
| | | | GGSEVQLVESGGGLVQPGGSLRLSCAAS |
| | | | GFNIKDTYIHWVRQAPGKGLEWVARIYP |
| | | | TNGYTRYADSVKGRFTISADTSKNTAYL |
| | | | QMNSLRAEDTAVYYCSRWGGDGFYAMDY |
| | | | WGQGTLVTVSSASTKGPSVFPLAPSSKS |
| | | | TSGGTAALGCLVKDYFPEPVTVSWNSGA |
| | | | LTSGVHTFPAVLQSSGLYSLSSVVTVPS |
| | | | SSLGTQTYICNVNHKPSNTKVDKKVEPK |
| | | | SCDKTHTCPPCPAPELLGGPSVFLFPPK |
| | | | PKDTLMISRTPEVTCVVVDVSHEDPEVK |
| | | | FNWYVDGVEVHNAKTKPREEQYASTYRV |
| | | | VSVLTVLHQDWLNGKEYKCKVSNKALPA |
| | | | PIEKTISKAKGQPREPQVYTLPPSRDEL |
| | | | TKNQVSLYCLVKGFYPSDIAVEWESNGQ |
| | | | PENNYKTTPPVLDSDGSFFLYSKLTVDK |
| | | | SRWQQGNVFSCSVMHEALHNHYTQKSLS |
| | | | LSPG |
| 36. | HC | Anti-Her2 Fc Hole, Y407T | EVQLVESGGGLVQPGGSLRLSCAASGFN |
| | | | IKDTYIHWVRQAPGKGLEWVARIYPTNG |
| | | | YTRYADSVKGRFTISADTSKNTAYLQMN |
| | | | SLRAEDTAVYYCSRWGGDGFYAMDYWGQ |
| | | | GTLVTVSSASTKGPSVFPLAPSSKSTSG |
| | | | GTAALGCLVKDYFPEPVTVSWNSGALTS |
| | | | GVHTFPAVLQSSGLYSLSSVVTVPSSSL |
| | | | GTQTYICNVNHKPSNTKVDKKVEPKSDK |
| | | | THTCPPCPAPELLGGPSVFLFPPKPKDT |
| | | | LMISRTPEVTCVVVDVSHEDPEVKFNWY |
| | | | VDGVEVHNAKTKPREEQYASTYRVVSVL |
| | | | TVLHQDWLNGKEYKCKVSNKALPAPIEK |
| | | | TISKAKGQPREPQVYTLPPSRDELTKNQ |
| | | | VSLTCLVKGFYPSDIAVEWESNGQPENN |
| | | | YKTTPPVLDSDGSFFLTSKLTVDKSRWQ |
| | | | QGNVFSCSVMHEALHNHYTQKSLSLSPG |
| 37. | Control | VH(anti-Her2)-(G4S)3- | EVQLVESGGGLVQPGGSLRLSCAASGFN |
| | Tandom | VL(anti-Her2)-SGGGGS- | IKDTYIHWVRQAPGKGLEWVARIYPTNG |
| | scFv | VH(SP34 HC5)- | YTRYADSVKGRFTISADTSKNTAYLQMN |
| | Sequence | VEGGSGGSGGSGGSGGV- | SLRAEDTAVYYCSRWGGDGFYAMDYWGQ |
| | | VL(SP34 LC6)-HHHHHH | GTLVTVSSGGGGSGGGGSGGGGSDIQMT |
| | | | QSPSSLSASVGDRVTITCRASQDVNTAV |
| | | | AWYQQKPGKAPKLLIYSASFLYSGVPSR |
| | | | FSGSRSGTDFTLTISSLQPEDFATYYCQ |
| | | | QHYTTPPTFGQGTKVEIKSGGGGSEVQL |
| | | | VESGGGLVQPGGSLRLSCAASGFTFNTY |
| | | | AMNWVRQAPGKGLEWVARIRSKYNNYAT |
| | | | YYADSVKGRFTVSRDDSKNTAYLQMNSL |
| | | | KTEDTAVYYCVRHGNFGNSYVSWFAYWG |
| | | | QGTMVTVSSVEGGSGGSGGSGGSGGVQT |
| | | | VVTQEPSLTVSPGGTVTLTCGSSTGAVT |
| | | | TSNYPNWVQQKPGQAPRGLIGGTNKKAP |
| | | | GTPARFSGSLLGGKAALTLSGAQPEDEA |
| | | | EYYCALWYSNLWVFGGGTKLTVLHHHHH |
| | | | H |
| 38. | HC | Anti-Her2 heavy chain | EVQLVESGGGLVQPGGSLRLSCAASGFN |
| | | Knob, T366Y | IKDTYIHWVRQAPGKGLEWVARIYPTNG |
| | | | YTRYADSVKGRFTISADTSKNTAYLQMN |
| | | | SLRAEDTAVYYCSRWGGDGFYAMDYWGQ |
| | | | GTLVTVSSASTKGPSVFPLAPSSKSTSG |
| | | | GTAALGCLVKDYFPEPVTVSWNSGALTS |
| | | | GVHTFPAVLQSSGLYSLSSVVTVPSSSL |
| | | | GTQTYICNVNHKPSNTKVDKKVEPKSCD |
| | | | KTHTCPPCPAPELLGGPSVFLFPPKPKD |
| | | | TLMISRTPEVTCVVVDVSHEDPEVKFNW |
| | | | YVDGVEVHNAKTKPREEQYASTYRVVSV |
| | | | LTVLHQDWLNGKEYKCKVSNKALPAPIE |
| | | | KTISKAKGQPREPQVYTLPPSRDELTKN |
| | | | QVSLYCLVKGFYPSDIAVEWESNGQPEN |
| | | | NYKTTPPVLDSDGSFFLYSKLTVDKSRW |
| | | | QQGNVFSCSVMHEALHNHYTQKSLSLSP |
| | | | G |
| 39. | HC | Heavy Chain (SP34 | EVQLVESGGGLVQPGGSLRLSCAASGFT |
| | | scFv(HC5 + LC6) G4AG4 Fc | FNTYAMNWVRQAPGKGLEWVARIRSKYN |
| | | Hole, Y407T) | NYATYYADSVKGRFTVSRDDSKNTAYLQ |
| | | | MNSLKTEDTAVYYCVRHGNFGNSYVSWF |
| | | | AYWGQGTMVTVSSGGGGSGGGGSGGGGS |
| | | | QTVVTQEPSLTVSPGGTVTLTCGSSTGA |
| | | | VTTSNYPNWVQQKPGQAPRGLIGGTNKK |
| | | | APGTPARFSGSLLGGKAALTLSGAQPED |
| | | | EAEYYCALWYSNLWVFGGGTKLTVL |
| | | | GGGGAGGGG |
| | | | DKTHTCPPCPAPELLGGPSVFLFPPKPK |
| | | | DTLMISRTPEVTCVVVDVSHEDPEVKFN |
| | | | WYVDGVEVHNAKTKPREEQYASTYRVVS |
| | | | VLTVLHQDWLNGKEYKCKVSNKALPAPI |
| | | | EKTISKAKGQPREPQVYTLPPSRDELTK |
| | | | NQVSLTCLVKGFYPSDIAVEWESNGQPE |
| | | | NNYKTTPPVLDSDGSFFLTSKLTVDKSR |
| | | | WQQGNVFSCSVMHEALHNHYTQKSLSLS |
| | | | PG |
| 40. | HC | SP34 scFv(HC5 + LC6) SG4S | EVQLVESGGGLVQPGGSLRLSCAASGFT |
| | | anti-Her2 heavy chain | FNTYAMNWVRQAPGKGLEWVARIRSKYN |
| | | Knob, T366Y | NYATYYADSVKGRFTVSRDDSKNTAYLQ |
| | | | MNSLKTEDTAVYYCVRHGNFGNSYVSWF |
| | | | AYWGQGTMVTVSSGGGGSGGGGSGGGGS |
| | | | QTVVTQEPSLTVSPGGTVTLTCGSSTGA |
| | | | VTTSNYPNWVQQKPGQAPRGLIGGTNKK |
| | | | APGTPARFSGSLLGGKAALTLSGAQPED |
| | | | EAEYYCALWYSNLWVFGGGTKLTVLSGG |
| | | | GGSEVQLVESGGGLVQPGGSLRLSCAAS |
| | | | GFNIKDTYIHWVRQAPGKGLEWVARIYP |
| | | | TNGYTRYADSVKGRFTISADTSKNTAYL |
| | | | QMNSLRAEDTAVYYCSRWGGDGFYAMDY |
| | | | WGQGTLVTVSSASTKGPSVFPLAPSSKS |
| | | | TSGGTAALGCLVKDYFPEPVTVSWNSGA |
| | | | LTSGVHTFPAVLQSSGLYSLSSVVTVPS |
| | | | SSLGTQTYICNVNHKPSNTKVDKKVEPK |
| | | | SCDKTHTCPPCPAPELLGGPSVFLFPPK |
| | | | PKDTLMISRTPEVTCVVVDVSHEDPEVK |
| | | | FNWYVDGVEVHNAKTKPREEQYASTYRV |
| | | | VSVLTVLHQDWLNGKEYKCKVSNKALPA |
| | | | PIEKTISKAKGQPREPQVYTLPPSRDEL |
| | | | TKNQVSLYCLVKGFYPSDIAVEWESNGQ |
| | | | PENNYKTTPPVLDSDGSFFLYSKLTVDK |
| | | | SRWQQGNVFSCSVMHEALHNHYTQKSLS |
| | | | LSPG |
| 41. | HC | Anti-Her2 Fc Hole, Y407T | EVQLVESGGGLVQPGGSLRLSCAASGFN |
| | | | IKDTYIHWVRQAPGKGLEWVARIYPTNG |
| | | | YTRYADSVKGRFTISADTSKNTAYLQMN |
| | | | SLRAEDTAVYYCSRWGGDGFYAMDYWGQ |
| | | | GTLVTVSSASTKGPSVFPLAPSSKSTSG |
| | | | GTAALGCLVKDYFPEPVTVSWNSGALTS |
| | | | GVHTFPAVLQSSGLYSLSSVVTVPSSSL |
| | | | GTQTYICNVNHKPSNTKVDKKVEPKSDK |
| | | | THTCPPCPAPELLGGPSVFLFPPKPKDT |
| | | | LMISRTPEVTCVVVDVSHEDPEVKFNWY |
| | | | VDGVEVHNAKTKPREEQYASTYRVVSVL |
| | | | TVLHQDWLNGKEYKCKVSNKALPAPIEK |
| | | | TISKAKGQPREPQVYTLPPSRDELTKNQ |
| | | | VSLTCLVKGFYPSDIAVEWESNGQPENN |
| | | | YKTTPPVLDSDGSFFLTSKLTVDKSRWQ |
| | | | QGNVFSCSVMHEALHNHYTQKSLSLSPG |
| 42. | Construct | anti-Her2 hIgG1 Knob | MEWSWVFLFFLSVTTGVHSEVQLVESGGGLV |
| | H7015 | (N297A, T366Y) | QPGGSLRLSCAASGFNIKDTYIHWVRQAPGK |
| | | | GLEWVARIYPTNGYTRYADSVKGRFTISADT |
| | | | SKNTAYLQMNSLRAEDTAVYYCSRWGGDGFY |
| | | | AMDYWGQGTLVTVSSASTKGPSVFPLAPSSK |
| | | | STSGGTAALGCLVKDYFPEPVTVSWNSGALT |
| | | | SGVHTFPAVLQSSGLYSLSSVVTVPSSSLGT |
| | | | QTYICNVNHKPSNTKVDKKVEPKSCDKTHTC |
| | | | PPCPAPELLGGPSVFLFPPKPKDTLMISRTP |
| | | | EVTCVVVDVSHEDPEVKFNWYVDGVEVHNAK |
| | | | TKPREEQYASTYRVVSVLTVLHQDWLNGKEY |
| | | | KCKVSNKALPAPIEKTISKAKGQPREPQVYT |
| | | | LPPSRDELTKNQVSLYCLVKGFYPSDIAVEW |
| | | | ESNGQPENNYKTTPPVLDSDGSFFLYSKLTV |
| | | | DKSRWQQGNVFSCSVMHEALHNHYTQKSLSL |
| | | | SPG* |
| 43. | Construct | SP34 scFv(HC4 + LC5)-hIgG1 | MEWSWVFLFFLSVTTGVHSEVQLVESGGGLV |
| | H7016 | Fc Hole (N297A, Y407T) | QPGGSLRLSCAASGFTFNTYAMNWVRQAPGK |
| | | | GLEWVARIRSKYNNYATYYADSVKDRFTVSR |
| | | | DDSKNTAYLQMNSLKTEDTAVYYCVRHGNFG |
| | | | NSYVSWFAYWGQGTMVTVSSGGGGSGGGGSG |
| | | | GGGSQTVVTQEPSLTVSPGGTVTLTCASSTG |
| | | | AVTTSNYANWVQQNPGQAPRGLIGGTNKKAP |
| | | | GTPARFSGSLLGGKAALTLSGVQPEDEAEYY |
| | | | CALWYSNLWVFGGGTKLTVLGGGGAGGGGDK |
| | | | THTCPPCPAPELLGGPSVFLFPPKPKDTLMI |
| | | | SRTPEVTCVVVDVSHEDPEVKFNWYVDGVEV |
| | | | HNAKTKPREEQYASTYRVVSVLTVLHQDWLN |
| | | | GKEYKCKVSNKALPAPIEKTISKAKGQPREP |
| | | | QVYTLPPSRDELTKNQVSLTCLVKGFYPSDI |
| | | | AVEWESNGQPENNYKTTPPVLDSDGSFFLTS |
| | | | KLTVDKSRWQQGNVFSCSVMHEALHNHYTQK |
| | | | SLSLSPG* |
| 44. | Construct | SP34 scFv(HC4 + LC5)-anti- | MEWSWVFLFFLSVTTGVHSEVQLVESGGGLV |
| | H7017 | Her2 hIgG1 Knob (N297A, | QPGGSLRLSCAASGFTFNTYAMNWVRQAPGK |
| | | T366Y) | GLEWVARIRSKYNNYATYYADSVKDRFTVSR |
| | | | DDSKNTAYLQMNSLKTEDTAVYYCVRHGNFG |
| | | | NSYVSWFAYWGQGTMVTVSSGGGGSGGGGSG |
| | | | GGGSQTVVTQEPSLTVSPGGTVTLTCASSTG |
| | | | AVTTSNYANWVQQNPGQAPRGLIGGTNKKAP |
| | | | GTPARFSGSLLGGKAALTLSGVQPEDEAEYY |
| | | | CALWYSNLWVFGGGTKLTVLSGGGGSEVQLV |
| | | | ESGGGLVQPGGSLRLSCAASGFNIKDTYIHW |
| | | | VRQAPGKGLEWVARIYPTNGYTRYADSVKGR |
| | | | FTISADTSKNTAYLQMNSLRAEDTAVYYCSR |
| | | | WGGDGFYAMDYWGQGTLVTVSSASTKGPSVF |
| | | | PLAPSSKSTSGGTAALGCLVKDYFPEPVTVS |
| | | | WNSGALTSGVHTFPAVLQSSGLYSLSSVVTV |
| | | | PSSSLGTQTYICNVNHKPSNTKVDKKVEPKS |
| | | | CDKTHTCPPCPAPELLGGPSVFLFPPKPKDT |
| | | | LMISRTPEVTCVVVDVSHEDPEVKFNWYVDG |
| | | | VEVHNAKTKPREEQYASTYRVVSVLTVLHQD |
| | | | WLNGKEYKCKVSNKALPAPIEKTISKAKGQP |
| | | | REPQVYTLPPSRDELTKNQVSLYCLVKGFYP |
| | | | SDIAVEWESNGQPENNYKTTPPVLDSDGSFF |
| | | | LYSKLTVDKSRWQQGNVFSCSVMHEALHNHY |
| | | | TQKSLSLSPG* |
| 45. | Construct | anti-Her2 hIgG1 Hole | MEWSWVFLFFLSVTTGVHSEVQLVESGGGLV |
| | H7018 | (N297A, Y4074T) | QPGGSLRLSCAASGFNIKDTYIHWVRQAPGK |
| | | | GLEWVARIYPTNGYTRYADSVKGRFTISADT |
| | | | SKNTAYLQMNSLRAEDTAVYYCSRWGGDGFY |
| | | | AMDYWGQGTLVTVSSASTKGPSVFPLAPSSK |
| | | | STSGGTAALGCLVKDYFPEPVTVSWNSGALT |
| | | | SGVHTFPAVLQSSGLYSLSSVVTVPSSSLGT |
| | | | QTYICNVNHKPSNTKVDKKVEPKSCDKTHTC |
| | | | PPCPAPELLGGPSVFLFPPKPKDTLMISRTP |
| | | | EVTCVVVDVSHEDPEVKFNWYVDGVEVHNAK |
| | | | TKPREEQYASTYRVVSVLTVLHQDWLNGKEY |
| | | | KCKVSNKALPAPIEKTISKAKGQPREPQVYT |
| | | | LPPSRDELTKNQVSLTCLVKGFYPSDIAVEW |
| | | | ESNGQPENNYKTTPPVLDSDGSFFLTSKLTV |
| | | | DKSRWQQGNVFSCSVMHEALHNHYTQKSLSL |
| | | | SPG* |
| 46. | Construct | SP34 scFv(HC5 + LC6)-hIgG1 | MEWSWVFLFFLSVTTGVHSEVQLVESGGGLV |
| | H7019 | Fc Hole (N297A, Y407T) | QPGGSLRLSCAASGFTFNTYAMNWVRQAPGK |
| | | | GLEWVARIRSKYNNYATYYADSVKGRFTVSR |
| | | | DDSKNTAYLQMNSLKTEDTAVYYCVRHGNFG |
| | | | NSYVSWFAYWGQGTMVTVSSGGGGSGGGGSG |
| | | | GGGSQTVVTQEPSLTVSPGGTVTLTCGSSTG |
| | | | AVTTSNYPNWVQQKPGQAPRGLIGGTNKKAP |
| | | | GTPARFSGSLLGGKAALTLSGAQPEDEAEYY |
| | | | CALWYSNLWVFGGGTKLTVLGGGGAGGGGDK |
| | | | THTCPPCPAPELLGGPSVFLFPPKPKDTLMI |
| | | | SRTPEVTCVVVDVSHEDPEVKFNWYVDGVEV |
| | | | HNAKTKPREEQYASTYRVVSVLTVLHQDWLN |
| | | | GKEYKCKVSNKALPAPIEKTISKAKGQPREP |
| | | | QVYTLPPSRDELTKNQVSLTCLVKGFYPSDI |
| | | | AVEWESNGQPENNYKTTPPVLDSDGSFFLTS |
| | | | KLTVDKSRWQQGNVFSCSVMHEALHNHYTQK |
| | | | SLSLSPG* |
| 47. | Construct | SP34 scFv(HC5 + LC6)-anti- | MEWSWVFLFFLSVTTGVHSEVQLVESGGGLV |
| | H7020 | Her2 hIgG1 Knob (N297A, | QPGGSLRLSCAASGFTFNTYAMNWVRQAPGK |
| | | T366Y) | GLEWVARIRSKYNNYATYYADSVKGRFTVSR |
| | | | DDSKNTAYLQMNSLKTEDTAVYYCVRHGNFG |
| | | | NSYVSWFAYWGQGTMVTVSSGGGGSGGGGSG |
| | | | GGGSQTVVTQEPSLTVSPGGTVTLTCGSSTG |
| | | | AVTTSNYPNWVQQKPGQAPRGLIGGTNKKAP |
| | | | GTPARFSGSLLGGKAALTLSGAQPEDEAEYY |
| | | | CALWYSNLWVFGGGTKLTVLSGGGGSEVQLV |
| | | | ESGGGLVQPGGSLRLSCAASGFNIKDTYIHW |
| | | | VRQAPGKGLEWVARIYPTNGYTRYADSVKGR |
| | | | FTISADTSKNTAYLQMNSLRAEDTAVYYCSR |
| | | | WGGDGFYAMDYWGQGTLVTVSSASTKGPSVF |
| | | | PLAPSSKSTSGGTAALGCLVKDYFPEPVTVS |
| | | | WNSGALTSGVHTFPAVLQSSGLYSLSSVVTV |
| | | | PSSSLGTQTYICNVNHKPSNTKVDKKVEPKS |
| | | | CDKTHTCPPCPAPELLGGPSVFLFPPKPKDT |
| | | | LMISRTPEVTCVVVDVSHEDPEVKFNWYVDG |
| | | | VEVHNAKTKPREEQYASTYRVVSVLTVLHQD |
| | | | WLNGKEYKCKVSNKALPAPIEKTISKAKGQP |
| | | | REPQVYTLPPSRDELTKNQVSLYCLVKGFYP |
| | | | SDIAVEWESNGQPENNYKTTPPVLDSDGSFF |
| | | | LYSKLTVDKSRWQQGNVFSCSVMHEALHNHY |
| | | | TQKSLSLSPG* |
| 48. | Construct | Control Tandem Her2 | MEWSWVFLFFLSVTTGVHSEVQLVESGGGLV |
| | PP11515 | SP34 scFv(HC4 + LC5) | QPGGSLRLSCAASGFNIKDTYIHWVRQAPGK |
| | J1331 | | GLEWVARIYPTNGYTRYADSVKGRFTISADT |
| | (DNA, see | | SKNTAYLQMNSLRAEDTAVYYCSRWGGDGFY |
| | below) | | AMDYWGQGTLVTVSSGGGGSGGGGSGGGGSD |
| | | | IQMTQSPSSLSASVGDRVTITCRASQDVNTA |
| | | | VAWYQQKPGKAPKLLIYSASFLYSGVPSRFS |
| | | | GSRSGTDFTLTISSLQPEDFATYYCQQHYTT |
| | | | PPTFGQGTKVEIKSGGGGSEVQLVESGGGLV |
| | | | QPGGSLRLSCAASGFTFNTYAMNWVRQAPGK |
| | | | GLEWVARIRSKYNNYATYYADSVKDRFTVSR |
| | | | DDSKNTAYLQMNSLKTEDTAVYYCVRHGNFG |
| | | | NSYVSWFAYWGQGTMVTVSSVEGGSGGSGGS |
| | | | GGSGGVQTVVTQEPSLTVSPGGTVTLTCASS |
| | | | TGAVTTSNYANWVQQNPGQAPRGLIGGTNKK |
| | | | APGTPARFSGSLLGGKAALTLSGVQPEDEAE |
| | | | YYCALWYSNLWVEGGGIKLIVLHHHHHH* |
| 49. | Construct | Control Tandem Her2 | MEWSWVFLFFLSVTTGVHSEVQLVESGGGLV |
| | PP11731 | SP34 scFv(HC5 + LC6) | QPGGSLRLSCAASGFNIKDTYIHWVRQAPGK |
| | J1332 | | GLEWVARIYPTNGYTRYADSVKGRFTISADT |
| | (DNA, see | | SKNTAYLQMNSLRAEDTAVYYCSRWGGDGFY |
| | below) | | AMDYWGQGTLVTVSSGGGGSGGGGSGGGGSD |
| | | | IQMTQSPSSLSASVGDRVTITCRASQDVNTA |
| | | | VAWYQQKPGKAPKLLIYSASFLYSGVPSRFS |
| | | | GSRSGTDFTLTISSLQPEDFATYYCQQHYTT |
| | | | PPTFGQGTKVEIKSGGGGSEVQLVESGGGLV |
| | | | QPGGSLRLSCAASGFTFNTYAMNWVRQAPGK |
| | | | GLEWVARIRSKYNNYATYYADSVKGRFTVSR |
| | | | DDSKNTAYLQMNSLKTEDTAVYYCVRHGNFG |
| | | | NSYVSWFAYWGQGTMVTVSSVEGGSGGSGGS |
| | | | GGSGGVQTVVTQEPSLTVSPGGTVTLTCGSS |
| | | | TGAVTTSNYPNWVQQKPGQAPRGLIGGTNKK |
| | | | APGTPARFSGSLLGGKAALTLSGAQPEDEAE |
| | | | YYCALWYSNLWVEGGGIKLIVLHHHHHH* |
| 50. | Construct | pLEV123-anti-HER2- | METDTLLLWVLLLWVPGSTGDIQMTQSPSSL |
| | L7015 | hKappa | SASVGDRVTITCRASQDVNTAVAWYQQKPGK |
| | | | APKLLIYSASFLYSGVPSRFSGSRSGTDFTL |
| | | | TISSLQPEDFATYYCQQHYTTPPTFGQGTKV |
| | | | EIKRTVAAPSVFIFPPSDEQLKSGTASVVCL |
| | | | LNNFYPREAKVQWKVDNALQSGNSQESVTEQ |
| | | | DSKDSTYSLSSTLTLSKADYEKHKVYACEVT |
| | | | HQGLSSPVTKSFNRGEC* |
| 51. | Construct | 1:1 anti-Her2 SP34 | Polypeptide: H7015 (SEQ ID NO: |
| | PP11519 | (HC4 + LC5) | 42) + H7016 (SEQ ID NO: 43) + |
| | | | H7015 (SEQ ID NO: 42) |
| | | | DNA: H7015 (SEQ ID NO: 55) + |
| | | | H7016 (SEQ ID NO: 56) + H7015 |
| | | | (SEQ ID NO: 55) |
| 52. | Construct | 2:1 anti-Her2 SP34 | Polypeptide: H7017 (SEQ ID NO: |
| | PP11520 | (HC4 + LC5) | 44) + H7018 (SEQ ID NO: 45) + |
| | | | H7015 (SEQ ID NO: 42) |
| | | | DNA: H7017 (SEQ ID NO: 57) + |
| | | | H7018 (SEQ ID NO: 58) + H7015 |
| | | | (SEQ ID NO: 55) |
| 53 | Construct | 1:1 anti-Her2 SP34 | Polypeptide: H7020 (SEQ ID NO: |
| | PP11521 | (HC5 + LC6) | 47) + H7019 (SEQ ID NO: 46) + |
| | | | H7015 (SEQ ID NO: 42) |
| | | | DNA: H7020 (SEQ ID NO: 60) + |
| | | | H7019 (SEQ ID NO: 59) + H7015 |
| | | | (SEQ ID NO: 55) |
| 54. | Construct | 2:1 anti-Her2 SP34 | Polypeptide: H7020 (SEQ ID NO: |
| | PP11523 | (HC5 + LC6) | 47) + H7018 (SEQ ID NO: 45) + |
| | | | H7015 (SEQ ID NO: 42) |
| | | | DNA: H7020 (SEQ ID NO: 60) + |
| | | | H7018 (SEQ ID NO: 58) + H7015 |
| | | | (SEQ ID NO: 55) |
| 55. | H7015 | anti-Her2 hIgG1 Knob | ATGGAATGGAGCTGGGTCTTTCTCTTCTTCC |
| | Construct | (N297A, T366Y) | TGTCAGTAACGACTGGTGTCCACTCCGAGGT |
| | DNA | | GCAGCTGGTGGAGAGCGGCGGAGGCCTCGTG |
| | | | CAGCCCGGCGGATCTCTGCGGCTGAGCTGCG |
| | | | CCGCTAGCGGCTTCAACATCAAGGACACCTA |
| | | | CATCCACTGGGTGCGCCAGGCCCCCGGCAAG |
| | | | GGCCTGGAGTGGGTGGCCCGGATCTACCCCA |
| | | | CCAACGGCTACACCCGCTACGCCGACAGCGT |
| | | | GAAGGGCCGGTTCACCATCAGCGCCGACACC |
| | | | TCCAAGAACACCGCCTACCTGCAGATGAACA |
| | | | GCCTGCGCGCCGAGGACACCGCCGTGTACTA |
| | | | CTGCAGCCGGTGGGGCGGCGACGGATTCTAC |
| | | | GCCATGGACTACTGGGGACAGGGCACCCTGG |
| | | | TGACCGTGAGCAGCGCTAGCACCAAGGGCCC |
| | | | CAGCGTGTTCCCTCTGGCCCCCAGCAGCAAG |
| | | | AGCACCAGCGGCGGAACCGCCGCCCTGGGCT |
| | | | GCCTGGTGAAGGACTACTTCCCCGAGCCCGT |
| | | | GACCGTGTCCTGGAACAGCGGCGCTCTGACC |
| | | | AGCGGAGTGCACACCTTCCCTGCCGTGCTGC |
| | | | AGAGCAGCGGCCTGTACTCCCTGAGCAGCGT |
| | | | GGTGACCGTGCCCAGCAGCAGCCTGGGCACC |
| | | | CAGACCTACATCTGCAACGTGAACCACAAGC |
| | | | CCTCCAACACCAAGGTGGACAAGAAGGTGGA |
| | | | GCCTAAGAGCTGCGACAAGACCCACACCTGC |
| | | | CCTCCCTGCCCCGCCCCCGAGCTGCTGGGCG |
| | | | GACCCAGCGTGTTCCTGTTCCCTCCCAAGCC |
| | | | CAAGGACACCCTGATGATCAGCCGCACCCCC |
| | | | GAGGTGACCTGCGTGGTGGTGGACGTGAGCC |
| | | | ACGAGGACCCCGAGGTGAAGTTCAACTGGTA |
| | | | CGTGGACGGCGTGGAGGTGCACAACGCCAAG |
| | | | ACCAAGCCTCGGGAGGAGCAGTACGCATCCA |
| | | | CCTACCGCGTGGTGAGCGTGCTGACCGTGCT |
| | | | GCACCAGGACTGGCTGAACGGCAAGGAGTAC |
| | | | AAGTGCAAGGTGAGCAACAAGGCCCTGCCCG |
| | | | CTCCCATCGAGAAGACCATCAGCAAGGCCAA |
| | | | GGGCCAGCCCCGGGAGCCTCAGGTGTACACC |
| | | | CTGCCCCCCAGCCGCGACGAGCTGACCAAGA |
| | | | ACCAGGTGAGCCTGTACTGCCTGGTGAAGGG |
| | | | CTTCTACCCCTCCGACATCGCCGTGGAGTGG |
| | | | GAGAGCAACGGCCAGCCTGAGAACAACTACA |
| | | | AGACCACCCCTCCCGTGCTGGACAGCGACGG |
| | | | CAGCTTCTTCCTGTACAGCAAGCTGACCGTG |
| | | | GACAAGTCCCGGTGGCAGCAGGGCAACGTGT |
| | | | TCAGCTGCAGCGTGATGCACGAGGCCCTGCA |
| | | | CAACCACTACACCCAGAAGAGCCTGAGCCTG |
| | | | AGCCCCGGATAG |
| 56. | H7016 | SP34 scFv(HC4 + LC5)-hIgG1 | ATGGAATGGAGCTGGGTCTTTCTCTTCTTCC |
| | Construct | Fc Hole (N297A, Y407T) | TGTCAGTAACGACTGGTGTCCACTCCGAGGT |
| | DNA | | GCAGCTGGTGGAGAGCGGTGGCGGCCTGGTG |
| | | | CAACCTGGCGGTAGCTTGAGGTTGAGCTGCG |
| | | | CCGCAAGCGGCTTCACCTTCAACACCTACGC |
| | | | CATGAACTGGGTGCGCCAGGCCCCAGGCAAG |
| | | | GGCCTGGAGTGGGTGGCCAGGATCAGGAGCA |
| | | | AGTACAACAACTATGCCACCTACTACGCCGA |
| | | | CAGCGTGAAGGACAGGTTCACCGTCAGCAGG |
| | | | GACGACAGCAAGAACACCGCCTACCTGCAGA |
| | | | TGAACTCACTGAAGACCGAGGACACCGCAGT |
| | | | GTACTACTGCGTGAGGCATGGCAACTTCGGC |
| | | | AACAGCTACGTGAGCTGGTTCGCCTACTGGG |
| | | | GCCAGGGCACCATGGTGACCGTGAGCAGCGG |
| | | | TGGCGGAGGATCTGGCGGAGGCGGTAGTGGC |
| | | | GGTGGCGGATCTCAGACCGTGGTGACCCAGG |
| | | | AGCCTAGCCTGACCGTGAGCCCTGGCGGAAC |
| | | | CGTGACCCTTACCTGCGCCTCAAGCACCGGA |
| | | | GCAGTTACCACCAGCAACTACGCCAACTGGG |
| | | | TCCAGCAGAATCCCGGGCAAGCCCCCAGGGG |
| | | | CCTGATTGGCGGCACCAACAAGAAGGCCCCC |
| | | | GGGACCCCCGCTAGGTTCAGCGGCAGCCTGC |
| | | | TGGGCGGCAAGGCCGCACTGACCCTGTCCGG |
| | | | AGTCCAGCCCGAGGACGAGGCCGAGTACTAC |
| | | | TGCGCCCTGTGGTACAGCAACCTGTGGGTAT |
| | | | TTGGCGGTGGCACTAAATTGACCGTTCTGGG |
| | | | CGGAGGTGGTGCAGGAGGCGGTGGAGACAAG |
| | | | ACCCACACCTGCCCTCCCTGCCCCGCCCCCG |
| | | | AGCTGCTGGGCGGACCCAGCGTGTTCCTGTT |
| | | | CCCTCCCAAGCCCAAGGACACCCTGATGATC |
| | | | AGCCGCACCCCCGAGGTGACCTGCGTGGTGG |
| | | | TGGACGTGAGCCACGAGGACCCCGAGGTGAA |
| | | | GTTCAACTGGTACGTGGACGGCGTGGAGGTG |
| | | | CACAACGCCAAGACCAAGCCTCGGGAGGAGC |
| | | | AGTACGCATCCACCTACCGCGTGGTGAGCGT |
| | | | GCTGACCGTGCTGCACCAGGACTGGCTGAAC |
| | | | GGCAAGGAGTACAAGTGCAAGGTGAGCAACA |
| | | | AGGCCCTGCCCGCTCCCATCGAGAAGACCAT |
| | | | CAGCAAGGCCAAGGGCCAGCCCCGGGAGCCT |
| | | | CAGGTGTACACCCTGCCCCCCAGCCGCGACG |
| | | | AGCTGACCAAGAACCAGGTGAGCCTGACCTG |
| | | | CCTGGTGAAGGGCTTCTACCCCTCCGACATC |
| | | | GCCGTGGAGTGGGAGAGCAACGGCCAGCCTG |
| | | | AGAACAACTACAAGACCACCCCTCCCGTGCT |
| | | | GGACAGCGACGGCAGCTTCTTCCTGACTAGC |
| | | | AAGCTGACCGTGGACAAGTCCCGGTGGCAGC |
| | | | AGGGCAACGTGTTCAGCTGCAGCGTGATGCA |
| | | | CGAGGCCCTGCACAACCACTACACCCAGAAG |
| | | | AGCCTGAGCCTGAGCCCCGGATAG |
| 57. | H7017 | SP34 scFv(HC4 + LC5)-anti- | ATGGAATGGAGCTGGGTCTTTCTCTTCTTCC |
| | Construct | Her2 hIgG1 Knob (N297A, | TGTCAGTAACGACTGGTGTCCACTCCGAGGT |
| | DNA | T366Y) | GCAGCTGGTGGAGAGCGGTGGCGGCCTGGTG |
| | | | CAACCTGGCGGTAGCTTGAGGTTGAGCTGCG |
| | | | CCGCAAGCGGCTTCACCTTCAACACCTACGC |
| | | | CATGAACTGGGTGCGCCAGGCCCCAGGCAAG |
| | | | GGCCTGGAGTGGGTGGCCAGGATCAGGAGCA |
| | | | AGTACAACAACTATGCCACCTACTACGCCGA |
| | | | CAGCGTGAAGGACAGGTTCACCGTCAGCAGG |
| | | | GACGACAGCAAGAACACCGCCTACCTGCAGA |
| | | | TGAACTCACTGAAGACCGAGGACACCGCAGT |
| | | | GTACTACTGCGTGAGGCATGGCAACTTCGGC |
| | | | AACAGCTACGTGAGCTGGTTCGCCTACTGGG |
| | | | GCCAGGGCACCATGGTGACCGTGAGCAGCGG |
| | | | TGGCGGAGGATCTGGCGGAGGCGGTAGTGGC |
| | | | GGTGGCGGATCTCAGACCGTGGTGACCCAGG |
| | | | AGCCTAGCCTGACCGTGAGCCCTGGCGGAAC |
| | | | CGTGACCCTTACCTGCGCCTCAAGCACCGGA |
| | | | GCAGTTACCACCAGCAACTACGCCAACTGGG |
| | | | TCCAGCAGAATCCCGGGCAAGCCCCCAGGGG |
| | | | CCTGATTGGCGGCACCAACAAGAAGGCCCCC |
| | | | GGGACCCCCGCTAGGTTCAGCGGCAGCCTGC |
| | | | TGGGCGGCAAGGCCGCACTGACCCTGTCCGG |
| | | | AGTCCAGCCCGAGGACGAGGCCGAGTACTAC |
| | | | TGCGCCCTGTGGTACAGCAACCTGTGGGTAT |
| | | | TTGGCGGTGGCACTAAATTGACCGTTCTGAG |
| | | | CGGCGGAGGTGGTTCAGAGGTGCAGCTGGTG |
| | | | GAGAGCGGCGGAGGCCTCGTGCAGCCCGGCG |
| | | | GATCTCTGCGGCTGAGCTGCGCCGCTAGCGG |
| | | | CTTCAACATCAAGGACACCTACATCCACTGG |
| | | | GTGCGCCAGGCCCCCGGCAAGGGCCTGGAGT |
| | | | GGGTGGCCCGGATCTACCCCACCAACGGCTA |
| | | | CACCCGCTACGCCGACAGCGTGAAGGGCCGG |
| | | | TTCACCATCAGCGCCGACACCTCCAAGAACA |
| | | | CCGCCTACCTGCAGATGAACAGCCTGCGCGC |
| | | | CGAGGACACCGCCGTGTACTACTGCAGCCGG |
| | | | TGGGGCGGCGACGGATTCTACGCCATGGACT |
| | | | ACTGGGGACAGGGCACCCTGGTGACCGTGAG |
| | | | CAGCGCTAGCACCAAGGGCCCCAGCGTGTTC |
| | | | CCTCTGGCCCCCAGCAGCAAGAGCACCAGCG |
| | | | GCGGAACCGCCGCCCTGGGCTGCCTGGTGAA |
| | | | GGACTACTTCCCCGAGCCCGTGACCGTGTCC |
| | | | TGGAACAGCGGCGCTCTGACCAGCGGAGTGC |
| | | | ACACCTTCCCTGCCGTGCTGCAGAGCAGCGG |
| | | | CCTGTACTCCCTGAGCAGCGTGGTGACCGTG |
| | | | CCCAGCAGCAGCCTGGGCACCCAGACCTACA |
| | | | TCTGCAACGTGAACCACAAGCCCTCCAACAC |
| | | | CAAGGTGGACAAGAAGGTGGAGCCTAAGAGC |
| | | | TGCGACAAGACCCACACCTGCCCTCCCTGCC |
| | | | CCGCCCCCGAGCTGCTGGGCGGACCCAGCGT |
| | | | GTTCCTGTTCCCTCCCAAGCCCAAGGACACC |
| | | | CTGATGATCAGCCGCACCCCCGAGGTGACCT |
| | | | GCGTGGTGGTGGACGTGAGCCACGAGGACCC |
| | | | CGAGGTGAAGTTCAACTGGTACGTGGACGGC |
| | | | GTGGAGGTGCACAACGCCAAGACCAAGCCTC |
| | | | GGGAGGAGCAGTACGCATCCACCTACCGCGT |
| | | | GGTGAGCGTGCTGACCGTGCTGCACCAGGAC |
| | | | TGGCTGAACGGCAAGGAGTACAAGTGCAAGG |
| | | | TGAGCAACAAGGCCCTGCCCGCTCCCATCGA |
| | | | GAAGACCATCAGCAAGGCCAAGGGCCAGCCC |
| | | | CGGGAGCCTCAGGTGTACACCCTGCCCCCCA |
| | | | GCCGCGACGAGCTGACCAAGAACCAGGTGAG |
| | | | CCTGTACTGCCTGGTGAAGGGCTTCTACCCC |
| | | | TCCGACATCGCCGTGGAGTGGGAGAGCAACG |
| | | | GCCAGCCTGAGAACAACTACAAGACCACCCC |
| | | | TCCCGTGCTGGACAGCGACGGCAGCTTCTTC |
| | | | CTGTACAGCAAGCTGACCGTGGACAAGTCCC |
| | | | GGTGGCAGCAGGGCAACGTGTTCAGCTGCAG |
| | | | CGTGATGCACGAGGCCCTGCACAACCACTAC |
| | | | ACCCAGAAGAGCCTGAGCCTGAGCCCCGGAT |
| | | | AG |
| 58. | H7018 | anti-Her2 hIgG1 Hole | ATGGAATGGAGCTGGGTCTTTCTCTTCTTCC |
| | Construct | (N297A, Y4074T) | TGTCAGTAACGACTGGTGTCCACTCCGAGGT |
| | DNA | | GCAGCTGGTGGAGAGCGGCGGAGGCCTCGTG |
| | | | CAGCCCGGCGGATCTCTGCGGCTGAGCTGCG |
| | | | CCGCTAGCGGCTTCAACATCAAGGACACCTA |
| | | | CATCCACTGGGTGCGCCAGGCCCCCGGCAAG |
| | | | GGCCTGGAGTGGGTGGCCCGGATCTACCCCA |
| | | | CCAACGGCTACACCCGCTACGCCGACAGCGT |
| | | | GAAGGGCCGGTTCACCATCAGCGCCGACACC |
| | | | TCCAAGAACACCGCCTACCTGCAGATGAACA |
| | | | GCCTGCGCGCCGAGGACACCGCCGTGTACTA |
| | | | CTGCAGCCGGTGGGGCGGCGACGGATTCTAC |
| | | | GCCATGGACTACTGGGGACAGGGCACCCTGG |
| | | | TGACCGTGAGCAGCGCTAGCACCAAGGGCCC |
| | | | CAGCGTGTTCCCTCTGGCCCCCAGCAGCAAG |
| | | | AGCACCAGCGGCGGAACCGCCGCCCTGGGCT |
| | | | GCCTGGTGAAGGACTACTTCCCCGAGCCCGT |
| | | | GACCGTGTCCTGGAACAGCGGCGCTCTGACC |
| | | | AGCGGAGTGCACACCTTCCCTGCCGTGCTGC |
| | | | AGAGCAGCGGCCTGTACTCCCTGAGCAGCGT |
| | | | GGTGACCGTGCCCAGCAGCAGCCTGGGCACC |
| | | | CAGACCTACATCTGCAACGTGAACCACAAGC |
| | | | CCTCCAACACCAAGGTGGACAAGAAGGTGGA |
| | | | GCCTAAGAGCTGCGACAAGACCCACACCTGC |
| | | | CCTCCCTGCCCCGCCCCCGAGCTGCTGGGCG |
| | | | GACCCAGCGTGTTCCTGTTCCCTCCCAAGCC |
| | | | CAAGGACACCCTGATGATCAGCCGCACCCCC |
| | | | GAGGTGACCTGCGTGGTGGTGGACGTGAGCC |
| | | | ACGAGGACCCCGAGGTGAAGTTCAACTGGTA |
| | | | CGTGGACGGCGTGGAGGTGCACAACGCCAAG |
| | | | ACCAAGCCTCGGGAGGAGCAGTACGCATCCA |
| | | | CCTACCGCGTGGTGAGCGTGCTGACCGTGCT |
| | | | GCACCAGGACTGGCTGAACGGCAAGGAGTAC |
| | | | AAGTGCAAGGTGAGCAACAAGGCCCTGCCCG |
| | | | CTCCCATCGAGAAGACCATCAGCAAGGCCAA |
| | | | GGGCCAGCCCCGGGAGCCTCAGGTGTACACC |
| | | | CTGCCCCCCAGCCGCGACGAGCTGACCAAGA |
| | | | ACCAGGTGAGCCTGACCTGCCTGGTGAAGGG |
| | | | CTTCTACCCCTCCGACATCGCCGTGGAGTGG |
| | | | GAGAGCAACGGCCAGCCTGAGAACAACTACA |
| | | | AGACCACCCCTCCCGTGCTGGACAGCGACGG |
| | | | CAGCTTCTTCCTGACTAGCAAGCTGACCGTG |
| | | | GACAAGTCCCGGTGGCAGCAGGGCAACGTGT |
| | | | TCAGCTGCAGCGTGATGCACGAGGCCCTGCA |
| | | | CAACCACTACACCCAGAAGAGCCTGAGCCTG |
| | | | AGCCCCGGATAG |
| 59. | H7019 | SP34 scFv(HC5 + LC6)-hIgG1 | ATGGAATGGAGCTGGGTCTTTCTCTTCTTCC |
| | Construct | Fc Hole (N 297A, Y407T) | TGTCAGTAACGACTGGTGTCCACTCCGAGGT |
| | DNA | | GCAGCTGGTGGAGAGCGGTGGCGGCCTGGTG |
| | | | CAACCTGGCGGTAGCTTGAGGTTGAGCTGCG |
| | | | CCGCAAGCGGCTTCACCTTCAACACCTACGC |
| | | | CATGAACTGGGTGCGCCAGGCCCCAGGCAAG |
| | | | GGCCTGGAGTGGGTGGCCAGGATCAGGAGCA |
| | | | AGTACAACAACTATGCCACCTACTACGCCGA |
| | | | CAGCGTGAAGGGCAGGTTCACCGTCAGCAGG |
| | | | GACGACAGCAAGAACACCGCCTACCTGCAGA |
| | | | TGAACTCACTGAAGACCGAGGACACCGCAGT |
| | | | GTACTACTGCGTGAGGCATGGCAACTTCGGC |
| | | | AACAGCTACGTGAGCTGGTTCGCCTACTGGG |
| | | | GCCAGGGCACCATGGTGACCGTGAGCAGCGG |
| | | | TGGCGGAGGATCTGGCGGAGGCGGTAGTGGC |
| | | | GGTGGCGGATCTCAGACCGTGGTGACCCAGG |
| | | | AGCCTAGCCTGACCGTGAGCCCTGGCGGAAC |
| | | | CGTGACCCTTACCTGCGGCTCAAGCACCGGA |
| | | | GCAGTTACCACCAGCAACTACCCCAACTGGG |
| | | | TCCAGCAGAAACCCGGGCAAGCCCCCAGGGG |
| | | | CCTGATTGGCGGCACCAACAAGAAGGCCCCC |
| | | | GGGACCCCCGCTAGGTTCAGCGGCAGCCTGC |
| | | | TGGGCGGCAAGGCCGCACTGACCCTGTCCGG |
| | | | AGCCCAGCCCGAGGACGAGGCCGAGTACTAC |
| | | | TGCGCCCTGTGGTACAGCAACCTGTGGGTAT |
| | | | TTGGCGGTGGCACTAAATTGACCGTTCTGGG |
| | | | CGGAGGTGGTGCAGGAGGCGGTGGAGACAAG |
| | | | ACCCACACCTGCCCTCCCTGCCCCGCCCCCG |
| | | | AGCTGCTGGGCGGACCCAGCGTGTTCCTGTT |
| | | | CCCTCCCAAGCCCAAGGACACCCTGATGATC |
| | | | AGCCGCACCCCCGAGGTGACCTGCGTGGTGG |
| | | | TGGACGTGAGCCACGAGGACCCCGAGGTGAA |
| | | | GTTCAACTGGTACGTGGACGGCGTGGAGGTG |
| | | | CACAACGCCAAGACCAAGCCTCGGGAGGAGC |
| | | | AGTACGCATCCACCTACCGCGTGGTGAGCGT |
| | | | GCTGACCGTGCTGCACCAGGACTGGCTGAAC |
| | | | GGCAAGGAGTACAAGTGCAAGGTGAGCAACA |
| | | | AGGCCCTGCCCGCTCCCATCGAGAAGACCAT |
| | | | CAGCAAGGCCAAGGGCCAGCCCCGGGAGCCT |
| | | | CAGGTGTACACCCTGCCCCCCAGCCGCGACG |
| | | | AGCTGACCAAGAACCAGGTGAGCCTGACCTG |
| | | | CCTGGTGAAGGGCTTCTACCCCTCCGACATC |
| | | | GCCGTGGAGTGGGAGAGCAACGGCCAGCCTG |
| | | | AGAACAACTACAAGACCACCCCTCCCGTGCT |
| | | | GGACAGCGACGGCAGCTTCTTCCTGACTAGC |
| | | | AAGCTGACCGTGGACAAGTCCCGGTGGCAGC |
| | | | AGGGCAACGTGTTCAGCTGCAGCGTGATGCA |
| | | | CGAGGCCCTGCACAACCACTACACCCAGAAG |
| | | | AGCCTGAGCCTGAGCCCCGGATAG |
| 60. | H7020 | SP34 scFv(HC5 + LC6)-anti- | ATGGAATGGAGCTGGGTCTTTCTCTTCTTCC |
| | Construct | Her2 hIgG1 Knob (N297A, | TGTCAGTAACGACTGGTGTCCACTCCGAGGT |
| | DNA | T366Y) | GCAGCTGGTGGAGAGCGGTGGCGGCCTGGTG |
| | | | CAACCTGGCGGTAGCTTGAGGTTGAGCTGCG |
| | | | CCGCAAGCGGCTTCACCTTCAACACCTACGC |
| | | | CATGAACTGGGTGCGCCAGGCCCCAGGCAAG |
| | | | GGCCTGGAGTGGGTGGCCAGGATCAGGAGCA |
| | | | AGTACAACAACTATGCCACCTACTACGCCGA |
| | | | CAGCGTGAAGGGCAGGTTCACCGTCAGCAGG |
| | | | GACGACAGCAAGAACACCGCCTACCTGCAGA |
| | | | TGAACTCACTGAAGACCGAGGACACCGCAGT |
| | | | GTACTACTGCGTGAGGCATGGCAACTTCGGC |
| | | | AACAGCTACGTGAGCTGGTTCGCCTACTGGG |
| | | | GCCAGGGCACCATGGTGACCGTGAGCAGCGG |
| | | | TGGCGGAGGATCTGGCGGAGGCGGTAGTGGC |
| | | | GGTGGCGGATCTCAGACCGTGGTGACCCAGG |
| | | | AGCCTAGCCTGACCGTGAGCCCTGGCGGAAC |
| | | | CGTGACCCTTACCTGCGGCTCAAGCACCGGA |
| | | | GCAGTTACCACCAGCAACTACCCCAACTGGG |
| | | | TCCAGCAGAAACCCGGGCAAGCCCCCAGGGG |
| | | | CCTGATTGGCGGCACCAACAAGAAGGCCCCC |
| | | | GGGACCCCCGCTAGGTTCAGCGGCAGCCTGC |
| | | | TGGGCGGCAAGGCCGCACTGACCCTGTCCGG |
| | | | AGCCCAGCCCGAGGACGAGGCCGAGTACTAC |
| | | | TGCGCCCTGTGGTACAGCAACCTGTGGGTAT |
| | | | TTGGCGGTGGCACTAAATTGACCGTTCTGAG |
| | | | CGGCGGAGGTGGTTCAGAGGTGCAGCTGGTG |
| | | | GAGAGCGGCGGAGGCCTCGTGCAGCCCGGCG |
| | | | GATCTCTGCGGCTGAGCTGCGCCGCTAGCGG |
| | | | CTTCAACATCAAGGACACCTACATCCACTGG |
| | | | GTGCGCCAGGCCCCCGGCAAGGGCCTGGAGT |
| | | | GGGTGGCCCGGATCTACCCCACCAACGGCTA |
| | | | CACCCGCTACGCCGACAGCGTGAAGGGCCGG |
| | | | TTCACCATCAGCGCCGACACCTCCAAGAACA |
| | | | CCGCCTACCTGCAGATGAACAGCCTGCGCGC |
| | | | CGAGGACACCGCCGTGTACTACTGCAGCCGG |
| | | | TGGGGCGGCGACGGATTCTACGCCATGGACT |
| | | | ACTGGGGACAGGGCACCCTGGTGACCGTGAG |
| | | | CAGCGCTAGCACCAAGGGCCCCAGCGTGTTC |
| | | | CCTCTGGCCCCCAGCAGCAAGAGCACCAGCG |
| | | | GCGGAACCGCCGCCCTGGGCTGCCTGGTGAA |
| | | | GGACTACTTCCCCGAGCCCGTGACCGTGTCC |
| | | | TGGAACAGCGGCGCTCTGACCAGCGGAGTGC |
| | | | ACACCTTCCCTGCCGTGCTGCAGAGCAGCGG |
| | | | CCTGTACTCCCTGAGCAGCGTGGTGACCGTG |
| | | | CCCAGCAGCAGCCTGGGCACCCAGACCTACA |
| | | | TCTGCAACGTGAACCACAAGCCCTCCAACAC |
| | | | CAAGGTGGACAAGAAGGTGGAGCCTAAGAGC |
| | | | TGCGACAAGACCCACACCTGCCCTCCCTGCC |
| | | | CCGCCCCCGAGCTGCTGGGCGGACCCAGCGT |
| | | | GTTCCTGTTCCCTCCCAAGCCCAAGGACACC |
| | | | CTGATGATCAGCCGCACCCCCGAGGTGACCT |
| | | | GCGTGGTGGTGGACGTGAGCCACGAGGACCC |
| | | | CGAGGTGAAGTTCAACTGGTACGTGGACGGC |
| | | | GTGGAGGTGCACAACGCCAAGACCAAGCCTC |
| | | | GGGAGGAGCAGTACGCATCCACCTACCGCGT |
| | | | GGTGAGCGTGCTGACCGTGCTGCACCAGGAC |
| | | | TGGCTGAACGGCAAGGAGTACAAGTGCAAGG |
| | | | TGAGCAACAAGGCCCTGCCCGCTCCCATCGA |
| | | | GAAGACCATCAGCAAGGCCAAGGGCCAGCCC |
| | | | CGGGAGCCTCAGGTGTACACCCTGCCCCCCA |
| | | | GCCGCGACGAGCTGACCAAGAACCAGGTGAG |
| | | | CCTGTACTGCCTGGTGAAGGGCTTCTACCCC |
| | | | TCCGACATCGCCGTGGAGTGGGAGAGCAACG |
| | | | GCCAGCCTGAGAACAACTACAAGACCACCCC |
| | | | TCCCGTGCTGGACAGCGACGGCAGCTTCTTC |
| | | | CTGTACAGCAAGCTGACCGTGGACAAGTCCC |
| | | | GGTGGCAGCAGGGCAACGTGTTCAGCTGCAG |
| | | | CGTGATGCACGAGGCCCTGCACAACCACTAC |
| | | | ACCCAGAAGAGCCTGAGCCTGAGCCCCGGAT |
| | | | AG |
| 61. | J1331 | Control Tandem Her2 | ATGGAATGGAGCTGGGTCTTTCTCTTCTTCC |
| | Construct | SP34 scFv(HC4 + LC5) | TGTCAGTAACGACTGGTGTCCACTCCGAGGT |
| | DNA | | GCAGCTGGTGGAGAGCGGCGGAGGCCTCGTG |
| | PP11515 | | CAGCCCGGCGGATCTCTGCGGCTGAGCTGCG |
| | | | CCGCTAGCGGCTTCAACATCAAGGACACCTA |
| | | | CATCCACTGGGTGCGCCAGGCCCCCGGCAAG |
| | | | GGCCTGGAGTGGGTGGCCCGGATCTACCCCA |
| | | | CCAACGGCTACACCCGCTACGCCGACAGCGT |
| | | | GAAGGGCCGGTTCACCATCAGCGCCGACACC |
| | | | TCCAAGAACACCGCCTACCTGCAGATGAACA |
| | | | GCCTGCGCGCCGAGGACACCGCCGTGTACTA |
| | | | CTGCAGCCGGTGGGGCGGCGACGGATTCTAC |
| | | | GCCATGGACTACTGGGGACAGGGCACCCTGG |
| | | | TGACCGTGAGCAGCGGTGGCGGAGGATCTGG |
| | | | CGGAGGCGGTAGTGGCGGTGGCGGATCTGAC |
| | | | ATCCAGATGACCCAGAGCCCTTCCAGCCTGA |
| | | | GCGCCAGCGTGGGCGACCGGGTGACCATCAC |
| | | | CTGCCGCGCTAGCCAGGACGTGAACACCGCC |
| | | | GTGGCCTGGTACCAGCAGAAGCCCGGAAAGG |
| | | | CCCCCAAGCTGCTGATCTACTCTGCTAGCTT |
| | | | CCTGTACAGCGGCGTGCCCAGCCGGTTCAGC |
| | | | GGATCTCGCAGCGGCACCGACTTCACCCTGA |
| | | | CCATCAGCAGCCTGCAGCCTGAGGACTTCGC |
| | | | CACCTACTACTGCCAGCAGCACTACACCACG |
| | | | CCTCCCACCTTCGGACAGGGCACCAAGGTGG |
| | | | AGATCAAGAGCGGCGGAGGTGGTTCAGAGGT |
| | | | GCAGCTGGTGGAGAGCGGTGGCGGCCTGGTG |
| | | | CAACCTGGCGGTAGCTTGAGGTTGAGCTGCG |
| | | | CCGCAAGCGGCTTCACCTTCAACACCTACGC |
| | | | CATGAACTGGGTGCGCCAGGCCCCAGGCAAG |
| | | | GGCCTGGAGTGGGTGGCCAGGATCAGGAGCA |
| | | | AGTACAACAACTATGCCACCTACTACGCCGA |
| | | | CAGCGTGAAGGACAGGTTCACCGTCAGCAGG |
| | | | GACGACAGCAAGAACACCGCCTACCTGCAGA |
| | | | TGAACTCACTGAAGACCGAGGACACCGCAGT |
| | | | GTACTACTGCGTGAGGCATGGCAACTTCGGC |
| | | | AACAGCTACGTGAGCTGGTTCGCCTACTGGG |
| | | | GCCAGGGCACCATGGTGACCGTGAGCAGCGT |
| | | | GGAGGGCGGCTCCGGGGGATCAGGCGGCAGT |
| | | | GGCGGAAGCGGCGGAGTTCAGACCGTGGTGA |
| | | | CCCAGGAGCCTAGCCTGACCGTGAGCCCTGG |
| | | | CGGAACCGTGACCCTTACCTGCGCCTCAAGC |
| | | | ACCGGAGCAGTTACCACCAGCAACTACGCCA |
| | | | ACTGGGTCCAGCAGAATCCCGGGCAAGCCCC |
| | | | CAGGGGCCTGATTGGCGGCACCAACAAGAAG |
| | | | GCCCCCGGGACCCCCGCTAGGTTCAGCGGCA |
| | | | GCCTGCTGGGCGGCAAGGCCGCACTGACCCT |
| | | | GTCCGGAGTCCAGCCCGAGGACGAGGCCGAG |
| | | | TACTACTGCGCCCTGTGGTACAGCAACCTGT |
| | | | GGGTATTTGGCGGTGGCACTAAATTGACCGT |
| | | | TCTGCACCACCATCATCACCATTAG |
| 62. | J1332 | Control Tandem Her2 | ATGGAATGGAGCTGGGTCTTTCTCTTCTTCC |
| | Construct | SP34 scFv(HC5 + LC6) | TGTCAGTAACGACTGGTGTCCACTCCGAGGT |
| | DNA | | GCAGCTGGTGGAGAGCGGCGGAGGCCTCGTG |
| | | | CAGCCCGGCGGATCTCTGCGGCTGAGCTGCG |
| | | | CCGCTAGCGGCTTCAACATCAAGGACACCTA |
| | | | CATCCACTGGGTGCGCCAGGCCCCCGGCAAG |
| | | | GGCCTGGAGTGGGTGGCCCGGATCTACCCCA |
| | | | CCAACGGCTACACCCGCTACGCCGACAGCGT |
| | | | GAAGGGCCGGTTCACCATCAGCGCCGACACC |
| | | | TCCAAGAACACCGCCTACCTGCAGATGAACA |
| | | | GCCTGCGCGCCGAGGACACCGCCGTGTACTA |
| | | | CTGCAGCCGGTGGGGCGGCGACGGATTCTAC |
| | | | GCCATGGACTACTGGGGACAGGGCACCCTGG |
| | | | TGACCGTGAGCAGCGGTGGCGGAGGATCTGG |
| | | | CGGAGGCGGTAGTGGCGGTGGCGGATCTGAC |
| | | | ATCCAGATGACCCAGAGCCCTTCCAGCCTGA |
| | | | GCGCCAGCGTGGGCGACCGGGTGACCATCAC |
| | | | CTGCCGCGCTAGCCAGGACGTGAACACCGCC |
| | | | GTGGCCTGGTACCAGCAGAAGCCCGGAAAGG |
| | | | CCCCCAAGCTGCTGATCTACTCTGCTAGCTT |
| | | | CCTGTACAGCGGCGTGCCCAGCCGGTTCAGC |
| | | | GGATCTCGCAGCGGCACCGACTTCACCCTGA |
| | | | CCATCAGCAGCCTGCAGCCTGAGGACTTCGC |
| | | | CACCTACTACTGCCAGCAGCACTACACCACG |
| | | | CCTCCCACCTTCGGACAGGGCACCAAGGTGG |
| | | | AGATCAAGAGCGGCGGAGGTGGTTCAGAGGT |
| | | | GCAGCTGGTGGAGAGCGGTGGCGGCCTGGTG |
| | | | CAACCTGGCGGTAGCTTGAGGTTGAGCTGCG |
| | | | CCGCAAGCGGCTTCACCTTCAACACCTACGC |
| | | | CATGAACTGGGTGCGCCAGGCCCCAGGCAAG |
| | | | GGCCTGGAGTGGGTGGCCAGGATCAGGAGCA |
| | | | AGTACAACAACTATGCCACCTACTACGCCGA |
| | | | CAGCGTGAAGGGCAGGTTCACCGTCAGCAGG |
| | | | GACGACAGCAAGAACACCGCCTACCTGCAGA |
| | | | TGAACTCACTGAAGACCGAGGACACCGCAGT |
| | | | GTACTACTGCGTGAGGCATGGCAACTTCGGC |
| | | | AACAGCTACGTGAGCTGGTTCGCCTACTGGG |
| | | | GCCAGGGCACCATGGTGACCGTGAGCAGCGT |
| | | | GGAGGGCGGCTCCGGGGGATCAGGCGGCAGT |
| | | | GGCGGAAGCGGCGGAGTTCAGACCGTGGTGA |
| | | | CCCAGGAGCCTAGCCTGACCGTGAGCCCTGG |
| | | | CGGAACCGTGACCCTTACCTGCGGCTCAAGC |
| | | | ACCGGAGCAGTTACCACCAGCAACTACCCCA |
| | | | ACTGGGTCCAGCAGAAACCCGGGCAAGCCCC |
| | | | CAGGGGCCTGATTGGCGGCACCAACAAGAAG |
| | | | GCCCCCGGGACCCCCGCTAGGTTCAGCGGCA |
| | | | GCCTGCTGGGCGGCAAGGCCGCACTGACCCT |
| | | | GTCCGGAGCCCAGCCCGAGGACGAGGCCGAG |
| | | | TACTACTGCGCCCTGTGGTACAGCAACCTGT |
| | | | GGGTATTTGGCGGTGGCACTAAATTGACCGT |
| | | | TCTGCACCACCATCATCACCATTAG |
| 63 | L7015 | pLEV123-anti-HER2- | ATGGAGACCGACACCCTGCTGCTCTGGGTGC |
| | Construct | hKappa | TGCTGCTCTGGGTGCCCGGCTCCACCGGAGA |
| | DNA | | CATCCAGATGACCCAGAGCCCTTCCAGCCTG |
| | | | AGCGCCAGCGTGGGCGACCGGGTGACCATCA |
| | | | CCTGCCGCGCTAGCCAGGACGTGAACACCGC |
| | | | CGTGGCCTGGTACCAGCAGAAGCCCGGAAAG |
| | | | GCCCCCAAGCTGCTGATCTACTCTGCTAGCT |
| | | | TCCTGTACAGCGGCGTGCCCAGCCGGTTCAG |
| | | | CGGATCTCGCAGCGGCACCGACTTCACCCTG |
| | | | ACCATCAGCAGCCTGCAGCCTGAGGACTTCG |
| | | | CCACCTACTACTGCCAGCAGCACTACACCAC |
| | | | GCCTCCCACCTTCGGACAGGGCACCAAGGTA |
| | | | GAGATCAAGCGGACCGTGGCCGCCCCCAGCG |
| | | | TGTTCATCTTCCCTCCCAGCGACGAGCAGCT |
| | | | GAAGTCTGGCACCGCCAGCGTGGTGTGCCTG |
| | | | CTGAACAACTTCTACCCCCGCGAGGCCAAGG |
| | | | TGCAGTGGAAGGTGGACAACGCCCTGCAGAG |
| | | | CGGCAACAGCCAGGAGAGCGTGACCGAGCAG |
| | | | GACTCCAAGGACAGCACCTACAGCCTGAGCA |
| | | | GCACCCTGACCCTGAGCAAGGCCGACTACGA |
| | | | GAAGCACAAGGTGTACGCCTGCGAGGTGACC |
| | | | CACCAGGGACTGTCTAGCCCCGTGACCAAGA |
| | | | GCTTCAACCGGGGCGAGTGCTAA |

### III. EXAMPLES

The following are examples of methods and compositions of the invention.

### Example 1: Humanization of SP34 CD3 Binding Antibodies Heavy Chain Domains

Humanization design of the parental antibody using in silico analyses was performed. Humanization began by generating a homology modeled antibody 3D structure. Acceptor frameworks based on overall sequence identity across the framework, matching interface position, similarly classed CDR canonical positions, and presence of N-glycosylation sites that would have to be removed were identified. Three light chain (LC) and two heavy chain (HC) frameworks were selected for humanization.

Humanized antibodies were designed by creating multiple hybrid sequences that fuse select parts of the parental antibody sequence with the human framework sequences. Using a 3D model, humanized sequences were methodically analyzed by eye and computer modeling to isolate sequences that would most likely retain antigen binding. The goal was to maximize the amount of human sequence in the final humanized antibodies while retaining original antibody specificity.

Three humanized heavy chains were designed based on two different heavy chain human acceptor frameworks.

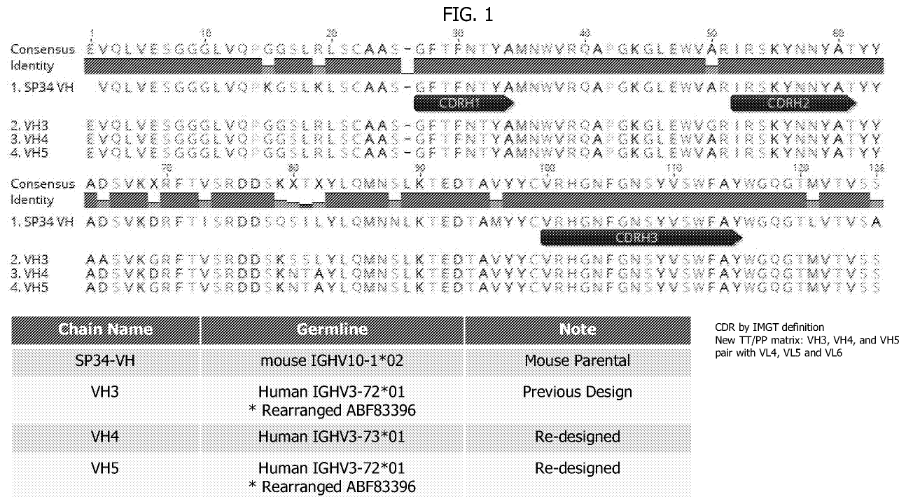

FIG. 1 shows a V_{H} consensus sequence on top followed by SP34 construct and then heavy chains V_{H}3, V_{H}4, and V_{H}5, according to embodiments of the invention.

### Example 2: Humanization of SP34 CD3 Binding Antibodies Light Chain Domains

Humanization design of parental antibody using in silico analyses was performed. Humanization began by generating a homology modeled antibody 3D structure. Acceptor frameworks were identified based on the overall sequence identity across the framework, matching interface position, similarly classed CDR canonical positions, and presence of N-glycosylation sites that would have to be removed.

Humanized antibodies were designed by creating multiple hybrid sequences that fuse select parts of the parental antibody sequence with the human framework sequences. Using a 3D model, humanized sequences were methodically analyzed by eye and computer modeling to isolate sequences that would most likely retain antigen binding. The goal was to maximize the amount of human sequence in the final humanized antibodies while retaining the original antibody specificity.

Three humanized light chains were designed based on three different light chain human acceptor frameworks.

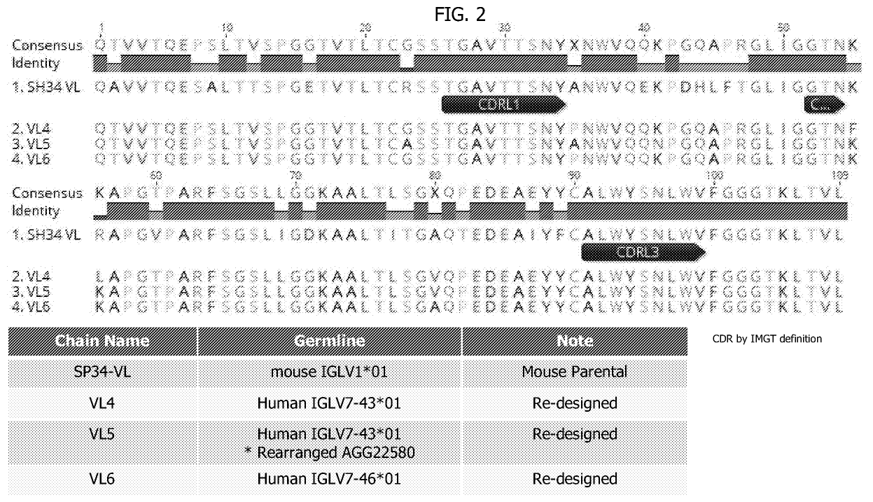

FIG. 2 shows a V_{L} consensus sequence on top followed by SP34 construct and then heavy chains V_{L}4, V_{L}5, and V_{H}6, according to embodiments of the invention.

### Example 3: Construction of Humanized CD3 Binding Portion of Bispecific Antibody

The humanized V_{H} and V_{L} sequences were codon optimized with a human codon bias. Short overlapping oligonucleotides spanning the entire gene sequence and complementary sequence were designed. The oligonucleotides were assembled via ligase chain reaction (LCR), whereby the two DNA strands were ligated to form double-stranded DNA fragments. The DNA fragments were then amplified by polymerase chain reaction (PCR), and each resulting PCR product was cloned into a mammalian vector via homologous recombination. Each completed construct was sequence confirmed before proceeding to DNA scale up.

Each DNA expression construct was scaled up to the appropriate amount for transfection. The plasmid DNA was run on agarose gel for quality assessment and sequence confirmed before proceeding to transfection.

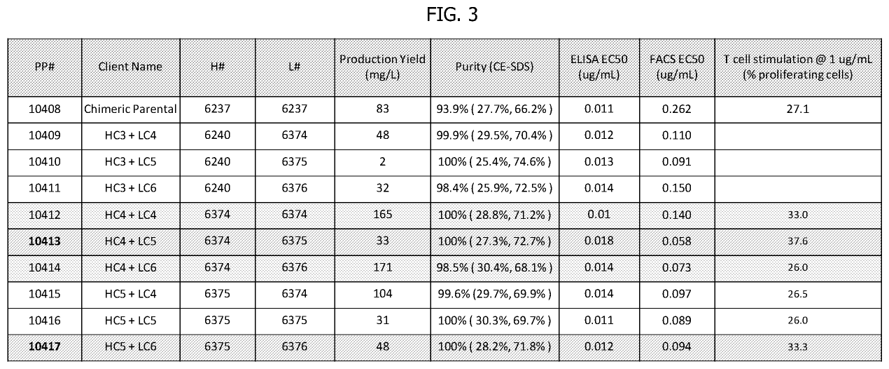

FIG. 3 summarizes information for constructs according to one embodiment of the invention. The left column sets forth the construct number. The next column sets forth individual components of the constructs. The next two columns set forth production and purification results, respectively. The last three columns set fort ELISA, FACS, and T-cell stimulation results, respectively, according to the invention.

### Example 4: Purification of Humanized CD3 Binding Portion of Bispecific Antibody

Suspension HEK293 cells were seeded in a shake flask and were expanded using serum-free chemically defined medium. On the day of transfection, the expanded cells were seeded into a new flask with fresh medium. Each DNA construct was transiently transfected into HEK293 cells. Cells were maintained as a batch-fed culture until the end of the production run.

The conditioned media from the transient production run was harvested and clarified by centrifugation and filtration. The supernatant was loaded over a Protein A column pre-equilibrated with binding buffer. Washing buffer was passed through the column until the OD280 value (NanoDrop, Thermo Scientific) was measured to be zero. The target protein was eluted with a low pH buffer, fractions were collected, and the OD280 value of each fraction was recorded. Fractions containing the target protein were pooled and filtered through a 0.2 μm membrane filter. The protein concentration was calculated from the OD280 value and the calculated extinction coefficient.

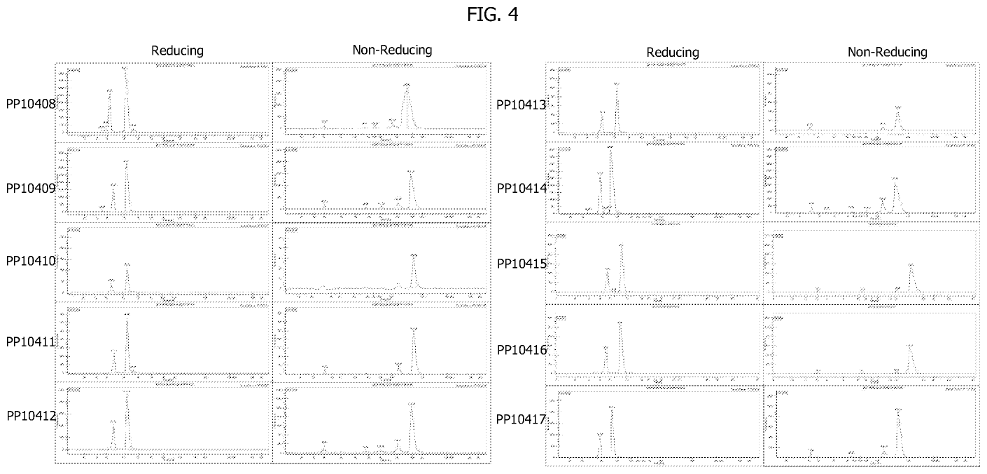

FIG. 4 shows a CE-SDS Electropherogram for respective antibody candidates. The left inset shows reducing conditions; the right inset shows non-reducing conditions. FIG. 4 shows protein purity, reduced and non-reduced. In general, all constructs show high levels of protein purity.

### Example 5: ELISA Binding Assay Against CD3 Heterodimer

The makeup of the constructs PP10408, PP10409, PP10410, PP10411, PP10412, PP10413, PP10414, PP10415, PP10416, and PP10417 is set forth in FIG. 3. CD3 heterodimer antigen was coated into wells at a concentration of 1 μg/mL overnight at 4° C. Each of PP10408, PP10409, PP10410, PP10411, PP10412, PP10413, PP10414, PP10415, PP10416, and PP10417 was added at a starting concentration of 10 μg/ml and diluted 1:4 at 11 points. A second antibody, HRP-anti-huFc, was added to the wells at a concentration of 1:10000. Blocking buffer of PBS+2% BSA was added. Wash buffer PBS-T was added. Results were read.

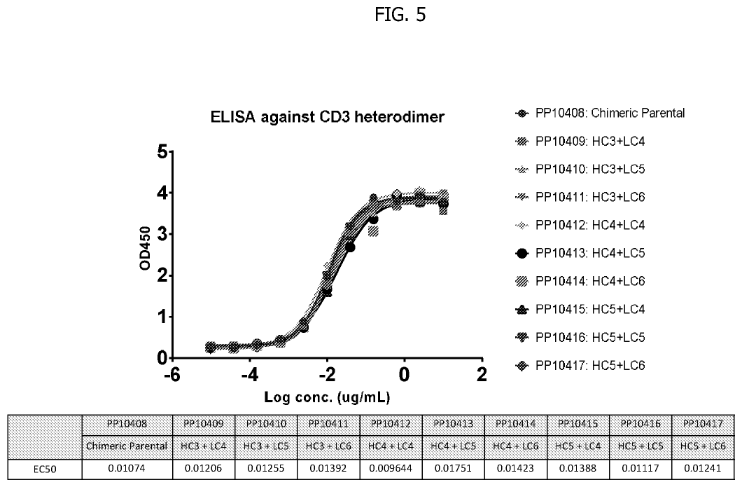

The results of the ELISA against a CD3 heterodimer can be seen in FIG. 5, with the x-axis showing Log conc. in g/ml and the y-axis showing OD450. EC₅₀s for each of PP10408, PP10409, PP10410, PP10411, PP10412, PP10413, PP10414, PP10415, PP10416, and PP10417 are shown in the inset at the bottom.

### Example 6: FACS Binding Assay Against CD3 Heterodimer

Each of the constructs PP10408, PP10409, PP10410, PP10411, PP10412, PP10413, PP10414, PP10415, PP10416, and PP10417 were also tested for binding in a fluorescence-activated cell sorting assay under the following conditions. 0.2E6 Jurkat cells were aliquoted into each well. 20 μg/ml of PP10408, PP10409, PP10410, PP10411, PP10412, PP10413, PP10414, PP10415, PP10416, and PP10417 were added and diluted 1:4 at 8 points. A second antibody, anti-human FITC, was added 1:200. FACS buffer of PBS+0.1% BSA+2 mM EDTA was added.

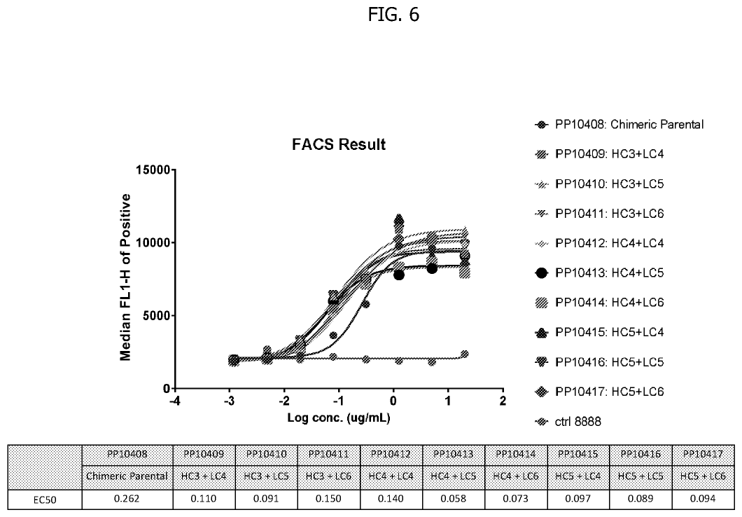

The results are shown in FIG. 6. EC₅₀s for each of PP10408, PP10409, PP10410, PP10411, PP10412, PP10413, PP10414, PP10415, PP10416, and PP10417 are shown in the inset at the bottom.

### Example 7: FACS Binding Assay Against PBMC

Each of the anti-CD3 anti-CD3 antibodies PP10408, PP10412, PP10413, PP10414, PP10415, PP10416, and PP10417 were assayed against PBMC.

The wells of a 96-well plate were coated with 0, 0.1, 1, and 10 μg/ml of each of PP10408, PP10412, PP10413, PP10414, PP10415, PP10416, and PP10417 and an isotype control (hulgG Fc only) over night at 4° C. PBMC cells were labelled with carboxyfluorescein succinimidyl ester (CFSE) the next day. The wells were rinsed and 100 of CFSE-labeled cells (2×10⁶/ml) was added to each well and cultured for 3 days. Wells were harvested and assessed for cell proliferation by flow cytometry.

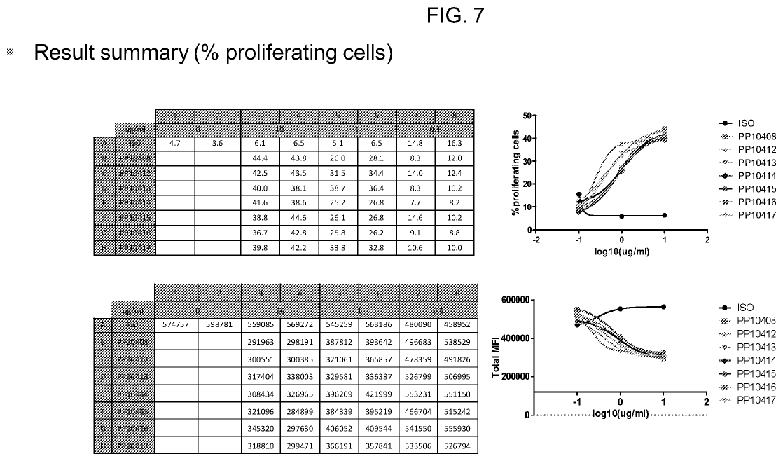

The results are shown in FIG. 7.

### Example 8: Construction of Anti-CD3 Bispecific Antibodies

Bispecific antibodies with any of constructs PP10408, PP10409, PP10410, PP10411, PP10412, PP10413, PP10414, PP10415, PP10416, and PP10417 and at least other antigen are constructed according to the methods set forth herein. A molecular weight of close to 150 kDa is preferred. Bispecific antibodies are constructed according to a knob-in-hole design (see, for example, Ridgeway et al, ‘Knobs-into-holes’ engineering of antibody CH3 domains for heavy chain heterodimerization Protein Engineering vol. 9 no. 7 pp. 617-621, 1996).

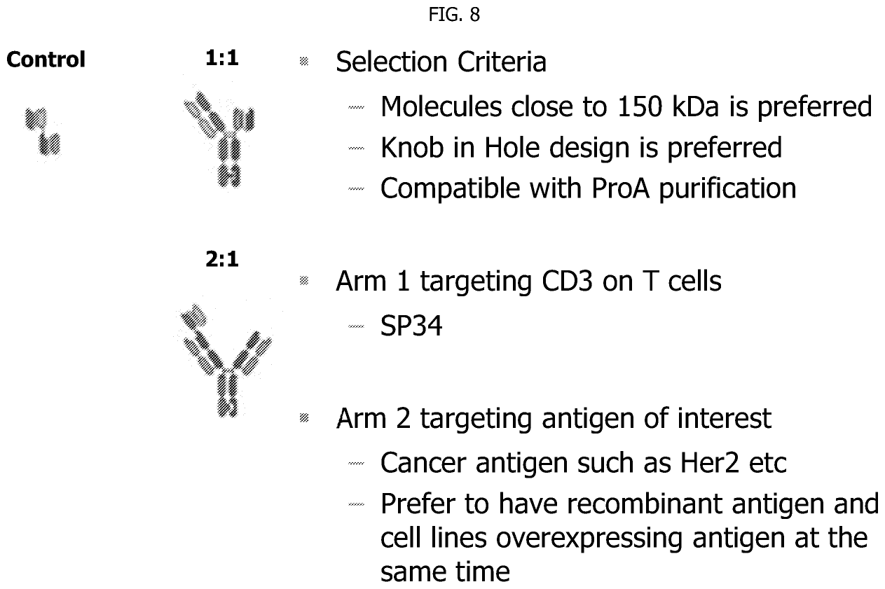

FIG. 8 shows construction of bispecific antibodies according to the invention. One of the binding sites is a humanized CD3 binding site, such as those set forth in the invention. For example, without limitation, at least one of PP10408, PP10409, PP10410, PP10411, PP10412, PP10413, PP10414, PP10415, PP10416, and PP10417 is used as a CD3 binding site. Another antigen is bound by a second binding site. Such an antigen may include, for example, without limitation, Her2 or any of the antigens set forth above.

In some embodiments, the bispecific antibody is a polypeptide comprising the amino acid sequence set forth in any one of SEQ ID NOs: 15, 20, 22, or 27.

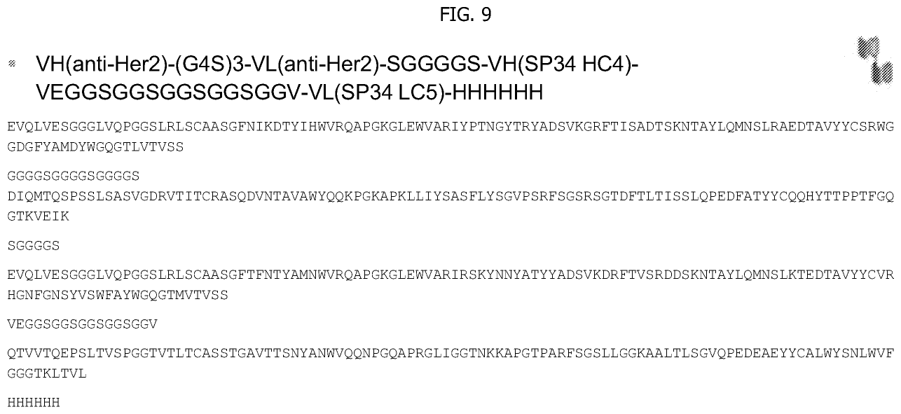

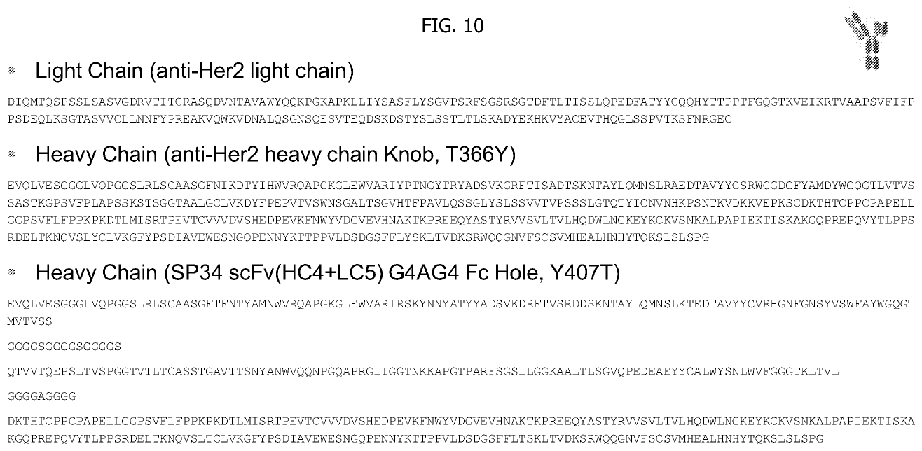

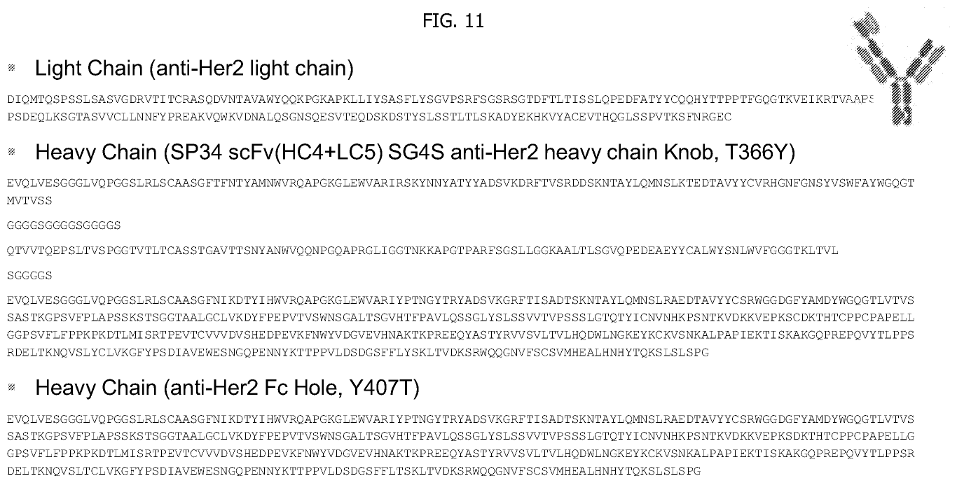

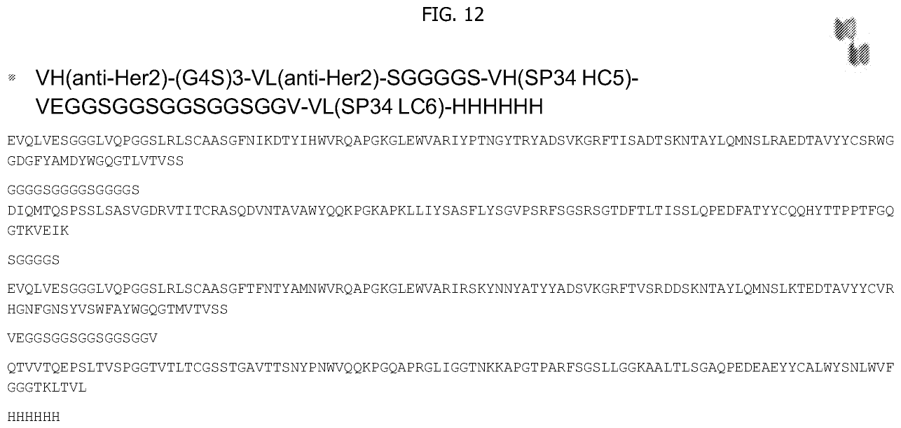

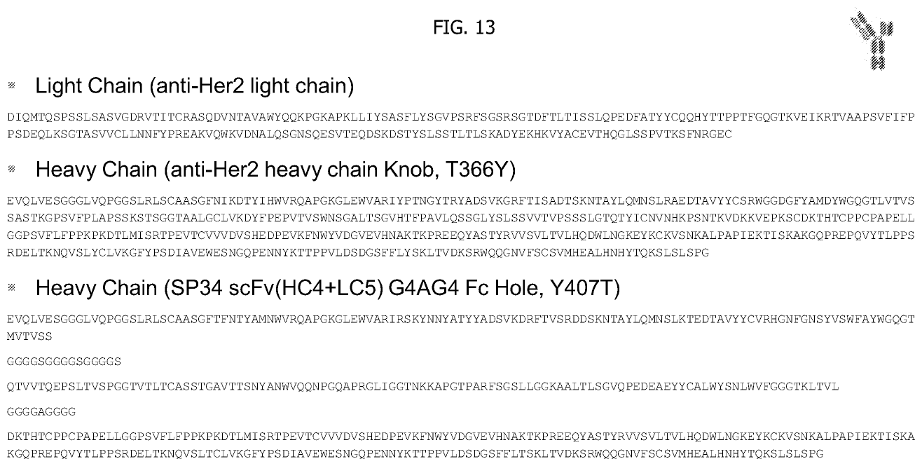

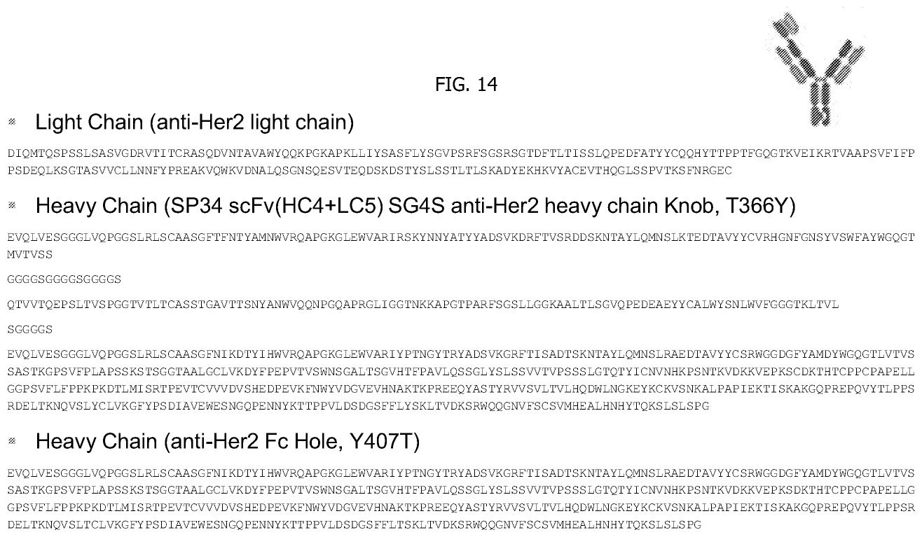

Constructs of the invention include those set forth in any of the figures for example, without limitation, the figures of FIG. 9, FIG. 10, FIG. 11, FIG. 12, FIG. 13, and FIG. 14.

### Example 9: Octet HTX Binding Assay for Antibodies Against the Her2 Extracellular Domain

Binding experiments were performed on an Octet HTX at 25° C. for each of PP11519, PP11520, PP11521, and PP11523 under the following conditions. Assay buffer was PBS with 0.1% BSA and 0.02% Tween20 at pH 7.2. Regeneration buffer was 10 mM glycine buffer at pH 1.7. Antibodies were loaded onto anti-human IgG Fc (AHC) biosensors. Loaded sensors were dipped into a three fold dilution of antigen (Her2), starting at 300 nM. Kinetic constants were calculated using a monovalent (1:1) binding model.

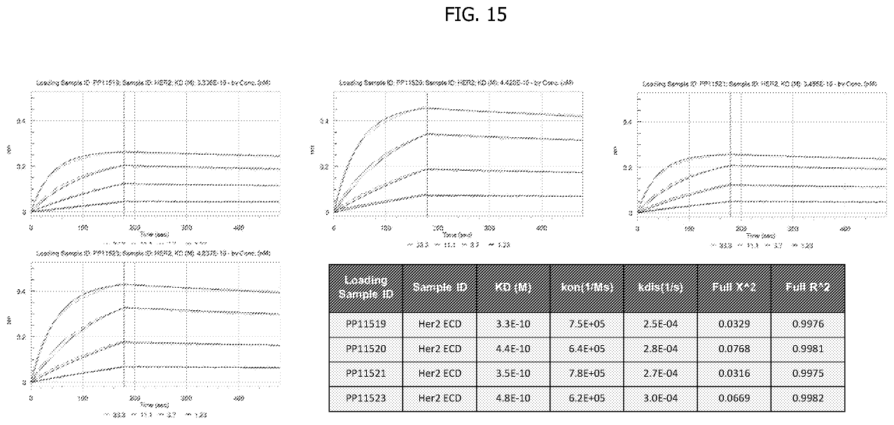

The results each for each of PP11519, PP11520, PP11521, and PP11523 are shown in FIG. 15. The inset shows a table setting forth loading sample ID, sample ID, KD, kon, kdis, FullX2, and Full R2.

### Example 10: Antibody Cytotoxicity

Antibodies were tested for cytotoxicity on SKBR3 and MCF7 cells. Specifically, antibodies PP11515, PP11731, PP11519, PP11520, PP11521, and PP11523 were tested. Antibodies PP11515 and PP11731 represent SP34 IgGs, as set forth in the insets. CFSE-labeled SKBR3 and MCF7 cells were seeded in a U-bottom 96-well plate (5E4/well) and incubated with different concentrations of antibodies (10 μg/ml, 10× dilution, 8 dilutions) for 25 minutes in the incubator. 1.25E6 of freshly isolated PBMC cells from one donor (2664) was added to each well and incubated at 37° C. for 24 hours.

Cell stripper was added to release the cells from the plate and collect them in a new V-bottom 96 well plate the following day. The cells were washed once with FACS buffer. Cells were resuspended with FACS buffer and 5 g/ml propidium iodide and data was recorded with an iQue screener.

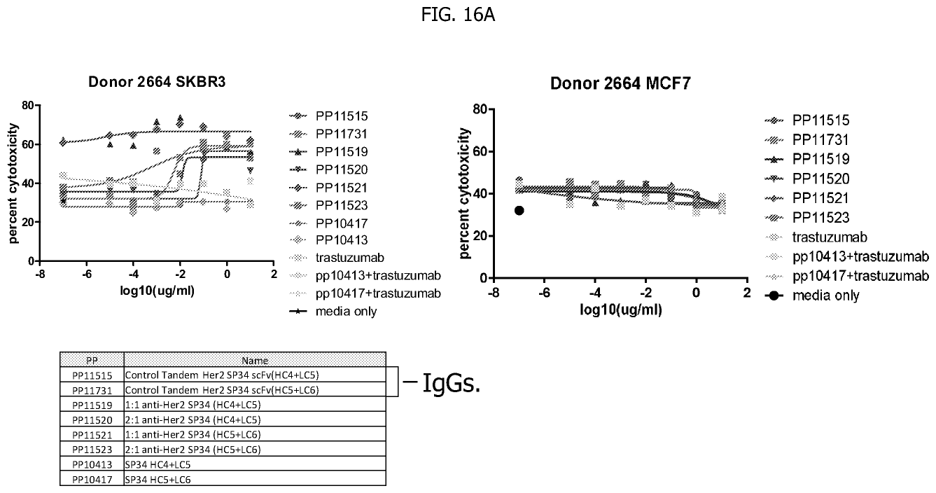

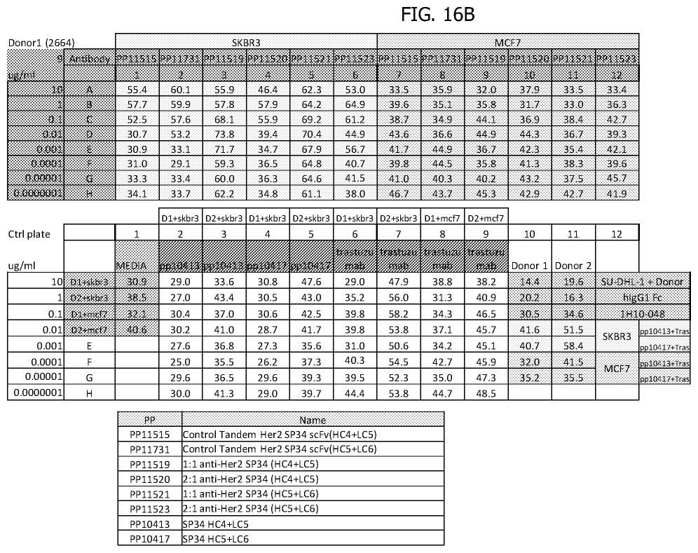

Data reported is the percentage of propidium idioide (PI) in CFSE positive cell populations. FIG. 16A depicts the % PI positive in CFSE positive cell populations in SKBR3 (left) and MCF7 (right) cells for Donor 1, 2664. The x-axis is log 10 (g/ml) and the y-axis is percent cytotoxicity. The inset depicts schematics for each of the respective constructs tested. FIG. 16B displays raw date for Donor 1, 2664.

### OTHER EMBODIMENTS

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A bispecific antibody that binds to CD3 and another antigen, comprising:
a first heavy chain binding domain comprising a polypeptide comprising an amino acid sequence of at least one of SEQ ID NOs: 1-4;
a second heavy chain binding domain comprising a polypeptide comprising an amino acid sequence of at least one of SEQ ID NOs: 15-19;
a first light chain binding domain comprising a polypeptide comprising an amino acid sequence of at least one of SEQ ID NOs: 8-11;
and a second light chain binding domain comprising a polypeptide comprising an amino acid sequence of at least one of SEQ ID NOs: 26-27.

2-5. (canceled)

6. The bispecific antibody of claim 1, wherein the bispecific antibody further comprises an Fc region.

7. The bispecific antibody of claim 6, wherein the Fc region comprises one or more substitution mutations that reduces effector function.

8. The bispecific antibody of claim 1, wherein the bispecific antibody is monoclonal, human, humanized, or chimeric.

9. The bispecific antibody of claim 1, wherein the bispecific antibody is a full-length antibody.

10. The bispecific antibody of claim 1, wherein the bispecific antibody is an IgA, an IgD, an IgE, an IgG, or an IgM antibody.

11-12. (canceled)

13. An isolated nucleic acid encoding the bispecific antibody of claim 1.

14. A vector comprising the isolated nucleic acid of claim 13.

15. A host cell comprising the vector of claim 14.

16-18. (canceled)

19. An immunoconjugate comprising the bispecific antibody of claim 1 and a cytotoxic agent.

20. A composition comprising the bispecific antibody of claim 1.

21. A method of treating or delaying the progression of a cell proliferative disorder or an autoimmune disorder in a subject, the method comprising administering to the subject an effective amount of the bispecific antibody of claim 1.

22. The method of claim 21, wherein the cell proliferative disorder is a cancer.

23. The method of claim 22, wherein the cancer is selected from the group consisting of breast cancer, colorectal cancer, non-small cell lung cancer, non-Hodgkin's lymphoma (NHL), B cell lymphoma, B cell leukemia, multiple myeloma, renal cancer, prostate cancer, liver cancer, head and neck cancer, melanoma, ovarian cancer, mesothelioma, glioblastoma, germinal-center B-cell-like (GCB) DLBCL, activated B-cell-like (ABC) DLBCL, follicular lymphoma (FL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), Waldenstrom macroglobulinemia (WM), central nervous system lymphoma (CNSL), Burkitt's lymphoma (BL), B-cell prolymphocytic leukemia, Splenic marginal zone lymphoma, Hairy cell leukemia, Splenic lymphoma/leukemia, unclassifiable, Splenic diffuse red pulp small B-cell lymphoma, Hairy cell leukemia variant, Waldenstrom macroglobulinemia, Heavy chain diseases, a Heavy chain disease, γ Heavy chain disease, Heavy chain disease, Plasma cell myeloma, Solitary plasmacytoma of bone, Extraosseous plasmacytoma, Extranodal marginal zone lymphoma of mucosa-associated lymphoid tissue (MALT lymphoma), Nodal marginal zone lymphoma, Pediatric nodal marginal zone lymphoma, Pediatric follicular lymphoma, Primary cutaneous follicle centre lymphoma, T-cell/histiocyte rich large B-cell lymphoma, Primary DLBCL of the CNS, Primary cutaneous DLBCL, leg type, EBV-positive DLBCL of the elderly, DLBCL associated with chronic inflammation, Lymphomatoid granulomatosis, Primary mediastinal (thymic) large B-cell lymphoma, Intravascular large B-cell lymphoma, ALK-positive large B-cell lymphoma, Plasmablastic lymphoma, Large B-cell lymphoma arising in HHV8-associated multicentric Castleman disease, Primary effusion lymphoma: B-cell lymphoma, unclassifiable, with features intermediate between diffuse large B-cell lymphoma and Burkitt lymphoma, and B-cell lymphoma, unclassifiable, with features intermediate between diffuse large B-cell lymphoma and classical Hodgkin lymphoma.

24. (canceled)

25. The method of claim 22, wherein the autoimmune disorder is selected from the group consisting of rheumatoid arthritis, juvenile rheumatoid arthritis, systemic lupus erythematosus (SLE), Wegener's disease, inflammatory bowel disease, idiopathic thrombocytopenic purpura (ITP), thrombotic thrombocytopenic purpura (TTP), autoimmune thrombocytopenia, multiple sclerosis, psoriasis, IgA nephropathy, IgM polyneuropathies, myasthenia gravis, vasculitis, diabetes mellitus, Reynaud's syndrome, Sjorgen's syndrome, glomerulonephritis, Neuromyelitis Optica (NMO), and IgG neuropathy

26. The bispecific antibody of claim 1, wherein the bispecific antibody is an antibody fragment.

27. The bispecific antibody of claim 26, wherein the antibody is selected from one or more an Fv fragment, a Fab fragment, a F(ab′)₂ fragment, a Fab′ fragment, an scFv (sFv) fragment, and an scFv-Fc fragment.

28. A pharmaceutical composition comprising the bispecific antibody of claim 1 and a pharmaceutically acceptable carrier.

29. A bispecific antibody that comprises more than one polypeptide comprising a combination of more than one polypeptide comprising an amino acid sequence of one or more of SEQ ID NO: 42, SEQ ID NO: 43, SEQ ID NO: 44. SEQ ID NO: 45, SEQ ID NO: 46, and/or SEQ ID NO: 47.

30. A bispecific antibody that comprises more than one polypeptide comprising more than one polypeptide comprising more than one amino acid sequence having at least 95%, 96%, 97%, 98%, or 99% to one or more of SEQ ID NO: 42, SEQ ID NO: 43, SEQ ID NO: 44. SEQ ID NO: 45, SEQ ID NO: 46, and/or SEQ ID NO: 47.


SEQUENCE LISTING (63 sequences)

SEQ ID NO: 1 - protein, 124 aa, Artificial Sequence
  Synthetic
VQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSA

SEQ ID NO: 2 - protein, 125 aa, Artificial Sequence
  Synthetic
EVQLVESGGGLVQPGGSLRLSCAASGFTFNTYAMNWVRQAPGKGLEWVGRIRSKYNNYATYYAASVKGRFTVSRDDSKSSLYLQMNSLKTEDTAVYYCVRHGNFGNSYVSWFAYWGQGTMVTVSS

SEQ ID NO: 3 - protein, 125 aa, Artificial Sequence
  Synthetic
EVQLVESGGGLVQPGGSLRLSCAASGFTFNTYAMNWVRQAPGKGLEWVARIRSKYNNYATYYADSVKDRFTVSRDDSKNTAYLQMNSLKTEDTAVYYCVRHGNFGNSYVSWFAYWGQGTMVTVSS

SEQ ID NO: 4 - protein, 125 aa, Artificial Sequence
  Synthetic
EVQLVESGGGLVQPGGSLRLSCAASGFTFNTYAMNWVRQAPGKGLEWVARIRSKYNNYATYYADSVKGRFTVSRDDSKNTAYLQMNSLKTEDTAVYYCVRHGNFGNSYVSWFAYWGQGTMVTVSS

SEQ ID NO: 5 - protein, 8 aa, Artificial Sequence
  Synthetic
GFTFNTYA

SEQ ID NO: 6 - protein, 10 aa, Artificial Sequence
  Synthetic
IRSKYNNYAT

SEQ ID NO: 7 - protein, 16 aa, Artificial Sequence
  Synthetic
VRHGNFGNSYVSWFAY

SEQ ID NO: 8 - protein, 109 aa, Artificial Sequence
  Synthetic
QAVVTQESALTTSPGETVTLTCRSSTGAVTTSNYANWVQEKPDHLFTGLIGGTNFRAPGVPARFSGSLIGDKAALTITGAQTEDEAIYFCALWYSNLWVFGGGTKLTVL

SEQ ID NO: 9 - protein, 109 aa, Artificial Sequence
  Synthetic
QTVVTQEPSLTVSPGGTVTLTCGSSTGAVTTSNYPNWVQQKPGQAPRGLIGGTNFLAPGTPARFSGSLLGGKAALTLSGVQPEDEAEYYCALWYSNLWVFGGGTKLTVL

SEQ ID NO: 10 - protein, 109 aa, Artificial Sequence
  Synthetic
QTVVTQEPSLTVSPGGTVTLTCASSTGAVTTSNYANWVQQNPGQAPRGLIGGTNKKAPGTPARFSGSLLGGKAALTLSGVQPEDEAEYYCALWYSNLWVFGGGTKLTVL

SEQ ID NO: 11 - protein, 109 aa, Artificial Sequence
  Synthetic
QTVVTQEPSLTVSPGGTVTLTCGSSTGAVTTSNYPNWVQQKPGQAPRGLIGGTNKKAPGTPARFSGSLLGGKAALTLSGAQPEDEAEYYCALWYSNLWVFGGGTKLTVL

SEQ ID NO: 12 - protein, 9 aa, Artificial Sequence
  Synthetic
TGAVTTSNY

SEQ ID NO: 13 - protein, 3 aa, Artificial Sequence
  Synthetic
GTN

SEQ ID NO: 14 - protein, 9 aa, Artificial Sequence
  Synthetic
ALWYSNLWV

SEQ ID NO: 15 - protein, 120 aa, Artificial Sequence [sequence omitted]

SEQ ID NO: 16 - protein, 449 aa, Artificial Sequence
  Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 125 aa, Artificial Sequence; identical to SEQ ID NO: 3
  Synthetic

SEQ ID NO: 18 - protein, 120 aa, Artificial Sequence [sequence omitted]

SEQ ID NO: 19 - protein, 125 aa, Artificial Sequence; identical to SEQ ID NO: 4
  Synthetic

SEQ ID NO: 20 - protein, 6 aa, Artificial Sequence
  Synthetic
IKDTYI

SEQ ID NO: 21 - protein, 6 aa, Artificial Sequence
  Synthetic
FNTYAM

SEQ ID NO: 22 - protein, 10 aa, Artificial Sequence
  Synthetic
RIYPTNGYTR

SEQ ID NO: 23 - protein, 10 aa, Artificial Sequence
  Synthetic
RIRSKYNNYA

SEQ ID NO: 24 - protein, 9 aa, Artificial Sequence
  Synthetic
WGGDGFYAM

SEQ ID NO: 25 - protein, 12 aa, Artificial Sequence
  Synthetic
HGNFGNSYVSWF

SEQ ID NO: 26 - protein, 107 aa, Artificial Sequence [sequence omitted]

SEQ ID NO: 27 - protein, 214 aa, Artificial Sequence
  Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 9 aa, Artificial Sequence
  Synthetic
SQDVNTAVA

SEQ ID NO: 29 - protein, 3 aa, Artificial Sequence
  Synthetic
SFL

SEQ ID NO: 30 - protein, 6 aa, Artificial Sequence
  Synthetic
HYTTPP

SEQ ID NO: 31 - protein, 505 aa, Artificial Sequence
  Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKSGGGGSEVQLVESGGGLVQPGGSLRLSCAASGFTFNTYAMNWVRQAPGKGLEWVARIRSKYNNYATYYADSVKDRFTVSRDDSKNTAYLQMNSLKTEDTAVYYCVRHGNFGNSYVSWFAYWGQGTMVTVSSVEGGSGGSGGSGGSGGVQTVVTQEPSLTVSPGGTVTLTCASSTGAVTTSNYANWVQQNPGQAPRGLIGGTNKKAPGTPARFSGSLLGGKAALTLSGVQPEDEAEYYCALWYSNLWVFGGGTKLTVLHHHHHH

SEQ ID NO: 32 - protein, 214 aa, Artificial Sequence; identical to SEQ ID NO: 27
  Synthetic

SEQ ID NO: 33 - protein, 449 aa, Artificial Sequence; identical to SEQ ID NO: 16
  Synthetic

SEQ ID NO: 34 - protein, 484 aa, Artificial Sequence
  Synthetic
EVQLVESGGGLVQPGGSLRLSCAASGFTFNTYAMNWVRQAPGKGLEWVARIRSKYNNYATYYADSVKDRFTVSRDDSKNTAYLQMNSLKTEDTAVYYCVRHGNFGNSYVSWFAYWGQGTMVTVSSGGGGSGGGGSGGGGSQTVVTQEPSLTVSPGGTVTLTCASSTGAVTTSNYANWVQQNPGQAPRGLIGGTNKKAPGTPARFSGSLLGGKAALTLSGVQPEDEAEYYCALWYSNLWVFGGGTKLTVLGGGGAGGGGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPG

SEQ ID NO: 35 - protein, 704 aa, Artificial Sequence
  Synthetic
EVQLVESGGGLVQPGGSLRLSCAASGFTFNTYAMNWVRQAPGKGLEWVARIRSKYNNYATYYADSVKDRFTVSRDDSKNTAYLQMNSLKTEDTAVYYCVRHGNFGNSYVSWFAYWGQGTMVTVSSGGGGSGGGGSGGGGSQTVVTQEPSLTVSPGGTVTLTCASSTGAVTTSNYANWVQQNPGQAPRGLIGGTNKKAPGTPARFSGSLLGGKAALTLSGVQPEDEAEYYCALWYSNLWVFGGGTKLTVLSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 448 aa, Artificial Sequence
  Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPG

SEQ ID NO: 37 - protein, 505 aa, Artificial Sequence
  Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKSGGGGSEVQLVESGGGLVQPGGSLRLSCAASGFTFNTYAMNWVRQAPGKGLEWVARIRSKYNNYATYYADSVKGRFTVSRDDSKNTAYLQMNSLKTEDTAVYYCVRHGNFGNSYVSWFAYWGQGTMVTVSSVEGGSGGSGGSGGSGGVQTVVTQEPSLTVSPGGTVTLTCGSSTGAVTTSNYPNWVQQKPGQAPRGLIGGTNKKAPGTPARFSGSLLGGKAALTLSGAQPEDEAEYYCALWYSNLWVFGGGTKLTVLHHHHHH

SEQ ID NO: 38 - protein, 449 aa, Artificial Sequence; identical to SEQ ID NO: 16
  Synthetic

SEQ ID NO: 39 - protein, 484 aa, Artificial Sequence
  Synthetic
EVQLVESGGGLVQPGGSLRLSCAASGFTFNTYAMNWVRQAPGKGLEWVARIRSKYNNYATYYADSVKGRFTVSRDDSKNTAYLQMNSLKTEDTAVYYCVRHGNFGNSYVSWFAYWGQGTMVTVSSGGGGSGGGGSGGGGSQTVVTQEPSLTVSPGGTVTLTCGSSTGAVTTSNYPNWVQQKPGQAPRGLIGGTNKKAPGTPARFSGSLLGGKAALTLSGAQPEDEAEYYCALWYSNLWVFGGGTKLTVLGGGGAGGGGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPG

SEQ ID NO: 40 - protein, 704 aa, Artificial Sequence
  Synthetic
EVQLVESGGGLVQPGGSLRLSCAASGFTFNTYAMNWVRQAPGKGLEWVARIRSKYNNYATYYADSVKGRFTVSRDDSKNTAYLQMNSLKTEDTAVYYCVRHGNFGNSYVSWFAYWGQGTMVTVSSGGGGSGGGGSGGGGSQTVVTQEPSLTVSPGGTVTLTCGSSTGAVTTSNYPNWVQQKPGQAPRGLIGGTNKKAPGTPARFSGSLLGGKAALTLSGAQPEDEAEYYCALWYSNLWVFGGGTKLTVLSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - protein, 448 aa, Artificial Sequence; identical to SEQ ID NO: 36
  Synthetic

SEQ ID NO: 42 - protein, 468 aa, Artificial Sequence
  Synthetic
MEWSWVFLFFLSVTTGVHSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 503 aa, Artificial Sequence
  Synthetic
MEWSWVFLFFLSVTTGVHSEVQLVESGGGLVQPGGSLRLSCAASGFTFNTYAMNWVRQAPGKGLEWVARIRSKYNNYATYYADSVKDRFTVSRDDSKNTAYLQMNSLKTEDTAVYYCVRHGNFGNSYVSWFAYWGQGTMVTVSSGGGGSGGGGSGGGGSQTVVTQEPSLTVSPGGTVTLTCASSTGAVTTSNYANWVQQNPGQAPRGLIGGTNKKAPGTPARFSGSLLGGKAALTLSGVQPEDEAEYYCALWYSNLWVFGGGTKLTVLGGGGAGGGGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPG

SEQ ID NO: 44 - protein, 723 aa, Artificial Sequence
  Synthetic
MEWSWVFLFFLSVTTGVHSEVQLVESGGGLVQPGGSLRLSCAASGFTFNTYAMNWVRQAPGKGLEWVARIRSKYNNYATYYADSVKDRFTVSRDDSKNTAYLQMNSLKTEDTAVYYCVRHGNFGNSYVSWFAYWGQGTMVTVSSGGGGSGGGGSGGGGSQTVVTQEPSLTVSPGGTVTLTCASSTGAVTTSNYANWVQQNPGQAPRGLIGGTNKKAPGTPARFSGSLLGGKAALTLSGVQPEDEAEYYCALWYSNLWVFGGGTKLTVLSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPG

SEQ ID NO: 45 - protein, 468 aa, Artificial Sequence
  Synthetic
MEWSWVFLFFLSVTTGVHSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPG

SEQ ID NO: 46 - protein, 503 aa, Artificial Sequence
  Synthetic
MEWSWVFLFFLSVTTGVHSEVQLVESGGGLVQPGGSLRLSCAASGFTFNTYAMNWVRQAPGKGLEWVARIRSKYNNYATYYADSVKGRFTVSRDDSKNTAYLQMNSLKTEDTAVYYCVRHGNFGNSYVSWFAYWGQGTMVTVSSGGGGSGGGGSGGGGSQTVVTQEPSLTVSPGGTVTLTCGSSTGAVTTSNYPNWVQQKPGQAPRGLIGGTNKKAPGTPARFSGSLLGGKAALTLSGAQPEDEAEYYCALWYSNLWVFGGGTKLTVLGGGGAGGGGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPG

SEQ ID NO: 47 - protein, 723 aa, Artificial Sequence
  Synthetic
MEWSWVFLFFLSVTTGVHSEVQLVESGGGLVQPGGSLRLSCAASGFTFNTYAMNWVRQAPGKGLEWVARIRSKYNNYATYYADSVKGRFTVSRDDSKNTAYLQMNSLKTEDTAVYYCVRHGNFGNSYVSWFAYWGQGTMVTVSSGGGGSGGGGSGGGGSQTVVTQEPSLTVSPGGTVTLTCGSSTGAVTTSNYPNWVQQKPGQAPRGLIGGTNKKAPGTPARFSGSLLGGKAALTLSGAQPEDEAEYYCALWYSNLWVFGGGTKLTVLSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 524 aa, Artificial Sequence
  Synthetic
MEWSWVFLFFLSVTTGVHSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKSGGGGSEVQLVESGGGLVQPGGSLRLSCAASGFTFNTYAMNWVRQAPGKGLEWVARIRSKYNNYATYYADSVKDRFTVSRDDSKNTAYLQMNSLKTEDTAVYYCVRHGNFGNSYVSWFAYWGQGTMVTVSSVEGGSGGSGGSGGSGGVQTVVTQEPSLTVSPGGTVTLTCASSTGAVTTSNYANWVQQNPGQAPRGLIGGTNKKAPGTPARFSGSLLGGKAALTLSGVQPEDEAEYYCALWYSNLWVFGGGTKLTVLHHHHHH

SEQ ID NO: 49 - protein, 524 aa, Artificial Sequence
  Synthetic
MEWSWVFLFFLSVTTGVHSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKSGGGGSEVQLVESGGGLVQPGGSLRLSCAASGFTFNTYAMNWVRQAPGKGLEWVARIRSKYNNYATYYADSVKGRFTVSRDDSKNTAYLQMNSLKTEDTAVYYCVRHGNFGNSYVSWFAYWGQGTMVTVSSVEGGSGGSGGSGGSGGVQTVVTQEPSLTVSPGGTVTLTCGSSTGAVTTSNYPNWVQQKPGQAPRGLIGGTNKKAPGTPARFSGSLLGGKAALTLSGAQPEDEAEYYCALWYSNLWVFGGGTKLTVLHHHHHH

SEQ ID NO: 50 - protein, 234 aa, Artificial Sequence
  Synthetic
METDTLLLWVLLLWVPGSTGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 1147 aa, Artificial Sequence
  Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGEVQLVESGGGLVQPGGSLRLSCAASGFTFNTYAMNWVRQAPGKGLEWVARIRSKYNNYATYYADSVKDRFTVSRDDSKNTAYLQMNSLKTEDTAVYYCVRHGNFGNSYVSWFAYWGQGTMVTVSSGGGGSGGGGSGGGGSQTVVTQEPSLTVSPGGTVTLTCASSTGAVTTSNYANWVQQNPGQAPRGLIGGTNKKAPGTPARFSGSLLGGKAALTLSGVQPEDEAEYYCALWYSNLWVFGGGTKLTVLGGGGAGGGGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPG

SEQ ID NO: 52 - protein, 1357 aa, Artificial Sequence
  Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGLVQPGGSLRLSCAASGFTFNTYAMNWVRQAPGKGLEWVARIRSKYNNYATYYADSVKDRFTVSRDDSKNTAYLQMNSLKTEDTAVYYCVRHGNFGNSYVSWFAYWGQGTMVTVSSGGGGSGGGGSGGGGSQTVVTQEPSLTVSPGGTVTLTCASSTGAVTTSNYANWVQQNPGQAPRGLIGGTNKKAPGTPARFSGSLLGGKAALTLSGVQPEDEAEYYCALWYSNLWVFGGGTKLTVLSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPG

SEQ ID NO: 53 - protein, 1147 aa, Artificial Sequence; identical to SEQ ID NO: 51
  Synthetic

SEQ ID NO: 54 - protein, 1366 aa, Artificial Sequence
  Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGLVQPGGSLRLSCAASGFTFNTYAMNWVRQAPGKGLEWVARIRSKYNNYATYYADSVKDRFTVSRDDSKNTAYLQMNSLKTEDTAVYYCVRHGNFGNSYVSWFAYWGQGTMVTVSSGGGGSGGGGSGGGGSQTVVTQEPSLTVSPGGTVTLTCASSTGAVTTSNYANWVQQNPGQAPRGLIGGTNKKAPGTPARFSGSLLGGKAALTLSGVQPEDEAEYYCALWYSNLWVFGGGTKLTVLSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPG

SEQ ID NO: 55 - DNA, 1407 bp, Artificial Sequence
  Synthetic
atggaatggagctgggtctttctcttcttcctgtcagtaacgactggtgtccactccgaggtgcagctggtggagagcggcggaggcctcgtgcagcccggcggatctctgcggctgagctgcgccgctagcggcttcaacatcaaggacacctacatccactgggtgcgccaggcccccggcaagggcctggagtgggtggcccggatctaccccaccaacggctacacccgctacgccgacagcgtgaagggccggttcaccatcagcgccgacacctccaagaacaccgcctacctgcagatgaacagcctgcgcgccgaggacaccgccgtgtactactgcagccggtggggcggcgacggattctacgccatggactactggggacagggcaccctggtgaccgtgagcagcgctagcaccaagggccccagcgtgttccctctggcccccagcagcaagagcaccagcggcggaaccgccgccctgggctgcctggtgaaggactacttccccgagcccgtgaccgtgtcctggaacagcggcgctctgaccagcggagtgcacaccttccctgccgtgctgcagagcagcggcctgtactccctgagcagcgtggtgaccgtgcccagcagcagcctgggcacccagacctacatctgcaacgtgaaccacaagccctccaacaccaaggtggacaagaaggtggagcctaagagctgcgacaagacccacacctgccctccctgccccgcccccgagctgctgggcggacccagcgtgttcctgttccctcccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagcctcgggaggagcagtacgcatccacctaccgcgtggtgagcgtgctgaccgtgctgcaccaggactggctgaacggcaaggagtacaagtgcaaggtgagcaacaaggccctgcccgctcccatcgagaagaccatcagcaaggccaagggccagccccgggagcctcaggtgtacaccctgccccccagccgcgacgagctgaccaagaaccaggtgagcctgtactgcctggtgaagggcttctacccctccgacatcgccgtggagtgggagagcaacggccagcctgagaacaactacaagaccacccctcccgtgctggacagcgacggcagcttcttcctgtacagcaagctgaccgtggacaagtcccggtggcagcagggcaacgtgttcagctgcagcgtgatgcacgaggccctgcacaaccactacacccagaagagcctgagcctgagccccggatag

SEQ ID NO: 56 - DNA, 1512 bp, Artificial Sequence
  Synthetic
atggaatggagctgggtctttctcttcttcctgtcagtaacgactggtgtccactccgaggtgcagctggtggagagcggtggcggcctggtgcaacctggcggtagcttgaggttgagctgcgccgcaagcggcttcaccttcaacacctacgccatgaactgggtgcgccaggccccaggcaagggcctggagtgggtggccaggatcaggagcaagtacaacaactatgccacctactacgccgacagcgtgaaggacaggttcaccgtcagcagggacgacagcaagaacaccgcctacctgcagatgaactcactgaagaccgaggacaccgcagtgtactactgcgtgaggcatggcaacttcggcaacagctacgtgagctggttcgcctactggggccagggcaccatggtgaccgtgagcagcggtggcggaggatctggcggaggcggtagtggcggtggcggatctcagaccgtggtgacccaggagcctagcctgaccgtgagccctggcggaaccgtgacccttacctgcgcctcaagcaccggagcagttaccaccagcaactacgccaactgggtccagcagaatcccgggcaagcccccaggggcctgattggcggcaccaacaagaaggcccccgggacccccgctaggttcagcggcagcctgctgggcggcaaggccgcactgaccctgtccggagtccagcccgaggacgaggccgagtactactgcgccctgtggtacagcaacctgtgggtatttggcggtggcactaaattgaccgttctgggcggaggtggtgcaggaggcggtggagacaagacccacacctgccctccctgccccgcccccgagctgctgggcggacccagcgtgttcctgttccctcccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagcctcgggaggagcagtacgcatccacctaccgcgtggtgagcgtgctgaccgtgctgcaccaggactggctgaacggcaaggagtacaagtgcaaggtgagcaacaaggccctgcccgctcccatcgagaagaccatcagcaaggccaagggccagccccgggagcctcaggtgtacaccctgccccccagccgcgacgagctgaccaagaaccaggtgagcctgacctgcctggtgaagggcttctacccctccgacatcgccgtggagtgggagagcaacggccagcctgagaacaactacaagaccacccctcccgtgctggacagcgacggcagcttcttcctgactagcaagctgaccgtggacaagtcccggtggcagcagggcaacgtgttcagctgcagcgtgatgcacgaggccctgcacaaccactacacccagaagagcctgagcctgagccccggatag

SEQ ID NO: 57 - DNA, 2172 bp, Artificial Sequence
  Synthetic
atggaatggagctgggtctttctcttcttcctgtcagtaacgactggtgtccactccgaggtgcagctggtggagagcggtggcggcctggtgcaacctggcggtagcttgaggttgagctgcgccgcaagcggcttcaccttcaacacctacgccatgaactgggtgcgccaggccccaggcaagggcctggagtgggtggccaggatcaggagcaagtacaacaactatgccacctactacgccgacagcgtgaaggacaggttcaccgtcagcagggacgacagcaagaacaccgcctacctgcagatgaactcactgaagaccgaggacaccgcagtgtactactgcgtgaggcatggcaacttcggcaacagctacgtgagctggttcgcctactggggccagggcaccatggtgaccgtgagcagcggtggcggaggatctggcggaggcggtagtggcggtggcggatctcagaccgtggtgacccaggagcctagcctgaccgtgagccctggcggaaccgtgacccttacctgcgcctcaagcaccggagcagttaccaccagcaactacgccaactgggtccagcagaatcccgggcaagcccccaggggcctgattggcggcaccaacaagaaggcccccgggacccccgctaggttcagcggcagcctgctgggcggcaaggccgcactgaccctgtccggagtccagcccgaggacgaggccgagtactactgcgccctgtggtacagcaacctgtgggtatttggcggtggcactaaattgaccgttctgagcggcggaggtggttcagaggtgcagctggtggagagcggcggaggcctcgtgcagcccggcggatctctgcggctgagctgcgccgctagcggcttcaacatcaaggacacctacatccactgggtgcgccaggcccccggcaagggcctggagtgggtggcccggatctaccccaccaacggctacacccgctacgccgacagcgtgaagggccggttcaccatcagcgccgacacctccaagaacaccgcctacctgcagatgaacagcctgcgcgccgaggacaccgccgtgtactactgcagccggtggggcggcgacggattctacgccatggactactggggacagggcaccctggtgaccgtgagcagcgctagcaccaagggccccagcgtgttccctctggcccccagcagcaagagcaccagcggcggaaccgccgccctgggctgcctggtgaaggactacttccccgagcccgtgaccgtgtcctggaacagcggcgctctgaccagcggagtgcacaccttccctgccgtgctgcagagcagcggcctgtactccctgagcagcgtggtgaccgtgcccagcagcagcctgggcacccagacctacatctgcaacgtgaaccacaagccctccaacaccaaggtggacaagaaggtggagcctaagagctgcgacaagacccacacctgccctccctgccccgcccccgagctgctgggcggacccagcgtgttcctgttccctcccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagcctcgggaggagcagtacgcatccacctaccgcgtggtgagcgtgctgaccgtgctgcaccaggactggctgaacggcaaggagtacaagtgcaaggtgagcaacaaggccctgcccgctcccatcgagaagaccatcagcaaggccaagggccagccccgggagcctcaggtgtacaccctgccccccagccgcgacgagctgaccaagaaccaggtgagcctgtactgcctggtgaagggcttctacccctccgacatcgccgtggagtgggagagcaacggccagcctgagaacaactacaagaccacccctcccgtgctggacagcgacggcagcttcttcctgtacagcaagctgaccgtggacaagtcccggtggcagcagggcaacgtgttcagctgcagcgtgatgcacgaggccctgcacaaccactacacccagaagagcctgagcctgagccccggatag

SEQ ID NO: 58 - DNA, 1407 bp, Artificial Sequence
  Synthetic
atggaatggagctgggtctttctcttcttcctgtcagtaacgactggtgtccactccgaggtgcagctggtggagagcggcggaggcctcgtgcagcccggcggatctctgcggctgagctgcgccgctagcggcttcaacatcaaggacacctacatccactgggtgcgccaggcccccggcaagggcctggagtgggtggcccggatctaccccaccaacggctacacccgctacgccgacagcgtgaagggccggttcaccatcagcgccgacacctccaagaacaccgcctacctgcagatgaacagcctgcgcgccgaggacaccgccgtgtactactgcagccggtggggcggcgacggattctacgccatggactactggggacagggcaccctggtgaccgtgagcagcgctagcaccaagggccccagcgtgttccctctggcccccagcagcaagagcaccagcggcggaaccgccgccctgggctgcctggtgaaggactacttccccgagcccgtgaccgtgtcctggaacagcggcgctctgaccagcggagtgcacaccttccctgccgtgctgcagagcagcggcctgtactccctgagcagcgtggtgaccgtgcccagcagcagcctgggcacccagacctacatctgcaacgtgaaccacaagccctccaacaccaaggtggacaagaaggtggagcctaagagctgcgacaagacccacacctgccctccctgccccgcccccgagctgctgggcggacccagcgtgttcctgttccctcccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagcctcgggaggagcagtacgcatccacctaccgcgtggtgagcgtgctgaccgtgctgcaccaggactggctgaacggcaaggagtacaagtgcaaggtgagcaacaaggccctgcccgctcccatcgagaagaccatcagcaaggccaagggccagccccgggagcctcaggtgtacaccctgccccccagccgcgacgagctgaccaagaaccaggtgagcctgacctgcctggtgaagggcttctacccctccgacatcgccgtggagtgggagagcaacggccagcctgagaacaactacaagaccacccctcccgtgctggacagcgacggcagcttcttcctgactagcaagctgaccgtggacaagtcccggtggcagcagggcaacgtgttcagctgcagcgtgatgcacgaggccctgcacaaccactacacccagaagagcctgagcctgagccccggatag

SEQ ID NO: 59 - DNA, 1512 bp, Artificial Sequence
  Synthetic
atggaatggagctgggtctttctcttcttcctgtcagtaacgactggtgtccactccgaggtgcagctggtggagagcggtggcggcctggtgcaacctggcggtagcttgaggttgagctgcgccgcaagcggcttcaccttcaacacctacgccatgaactgggtgcgccaggccccaggcaagggcctggagtgggtggccaggatcaggagcaagtacaacaactatgccacctactacgccgacagcgtgaagggcaggttcaccgtcagcagggacgacagcaagaacaccgcctacctgcagatgaactcactgaagaccgaggacaccgcagtgtactactgcgtgaggcatggcaacttcggcaacagctacgtgagctggttcgcctactggggccagggcaccatggtgaccgtgagcagcggtggcggaggatctggcggaggcggtagtggcggtggcggatctcagaccgtggtgacccaggagcctagcctgaccgtgagccctggcggaaccgtgacccttacctgcggctcaagcaccggagcagttaccaccagcaactaccccaactgggtccagcagaaacccgggcaagcccccaggggcctgattggcggcaccaacaagaaggcccccgggacccccgctaggttcagcggcagcctgctgggcggcaaggccgcactgaccctgtccggagcccagcccgaggacgaggccgagtactactgcgccctgtggtacagcaacctgtgggtatttggcggtggcactaaattgaccgttctgggcggaggtggtgcaggaggcggtggagacaagacccacacctgccctccctgccccgcccccgagctgctgggcggacccagcgtgttcctgttccctcccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagcctcgggaggagcagtacgcatccacctaccgcgtggtgagcgtgctgaccgtgctgcaccaggactggctgaacggcaaggagtacaagtgcaaggtgagcaacaaggccctgcccgctcccatcgagaagaccatcagcaaggccaagggccagccccgggagcctcaggtgtacaccctgccccccagccgcgacgagctgaccaagaaccaggtgagcctgacctgcctggtgaagggcttctacccctccgacatcgccgtggagtgggagagcaacggccagcctgagaacaactacaagaccacccctcccgtgctggacagcgacggcagcttcttcctgactagcaagctgaccgtggacaagtcccggtggcagcagggcaacgtgttcagctgcagcgtgatgcacgaggccctgcacaaccactacacccagaagagcctgagcctgagccccggatag

SEQ ID NO: 60 - DNA, 2172 bp, Artificial Sequence
  Synthetic
atggaatggagctgggtctttctcttcttcctgtcagtaacgactggtgtccactccgaggtgcagctggtggagagcggtggcggcctggtgcaacctggcggtagcttgaggttgagctgcgccgcaagcggcttcaccttcaacacctacgccatgaactgggtgcgccaggccccaggcaagggcctggagtgggtggccaggatcaggagcaagtacaacaactatgccacctactacgccgacagcgtgaagggcaggttcaccgtcagcagggacgacagcaagaacaccgcctacctgcagatgaactcactgaagaccgaggacaccgcagtgtactactgcgtgaggcatggcaacttcggcaacagctacgtgagctggttcgcctactggggccagggcaccatggtgaccgtgagcagcggtggcggaggatctggcggaggcggtagtggcggtggcggatctcagaccgtggtgacccaggagcctagcctgaccgtgagccctggcggaaccgtgacccttacctgcggctcaagcaccggagcagttaccaccagcaactaccccaactgggtccagcagaaacccgggcaagcccccaggggcctgattggcggcaccaacaagaaggcccccgggacccccgctaggttcagcggcagcctgctgggcggcaaggccgcactgaccctgtccggagcccagcccgaggacgaggccgagtactactgcgccctgtggtacagcaacctgtgggtatttggcggtggcactaaattgaccgttctgagcggcggaggtggttcagaggtgcagctggtggagagcggcggaggcctcgtgcagcccggcggatctctgcggctgagctgcgccgctagcggcttcaacatcaaggacacctacatccactgggtgcgccaggcccccggcaagggcctggagtgggtggcccggatctaccccaccaacggctacacccgctacgccgacagcgtgaagggccggttcaccatcagcgccgacacctccaagaacaccgcctacctgcagatgaacagcctgcgcgccgaggacaccgccgtgtactactgcagccggtggggcggcgacggattctacgccatggactactggggacagggcaccctggtgaccgtgagcagcgctagcaccaagggccccagcgtgttccctctggcccccagcagcaagagcaccagcggcggaaccgccgccctgggctgcctggtgaaggactacttccccgagcccgtgaccgtgtcctggaacagcggcgctctgaccagcggagtgcacaccttccctgccgtgctgcagagcagcggcctgtactccctgagcagcgtggtgaccgtgcccagcagcagcctgggcacccagacctacatctgcaacgtgaaccacaagccctccaacaccaaggtggacaagaaggtggagcctaagagctgcgacaagacccacacctgccctccctgccccgcccccgagctgctgggcggacccagcgtgttcctgttccctcccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagcctcgggaggagcagtacgcatccacctaccgcgtggtgagcgtgctgaccgtgctgcaccaggactggctgaacggcaaggagtacaagtgcaaggtgagcaacaaggccctgcccgctcccatcgagaagaccatcagcaaggccaagggccagccccgggagcctcaggtgtacaccctgccccccagccgcgacgagctgaccaagaaccaggtgagcctgtactgcctggtgaagggcttctacccctccgacatcgccgtggagtgggagagcaacggccagcctgagaacaactacaagaccacccctcccgtgctggacagcgacggcagcttcttcctgtacagcaagctgaccgtggacaagtcccggtggcagcagggcaacgtgttcagctgcagcgtgatgcacgaggccctgcacaaccactacacccagaagagcctgagcctgagccccggatag

SEQ ID NO: 61 - DNA, 1575 bp, Artificial Sequence
  Synthetic
atggaatggagctgggtctttctcttcttcctgtcagtaacgactggtgtccactccgaggtgcagctggtggagagcggcggaggcctcgtgcagcccggcggatctctgcggctgagctgcgccgctagcggcttcaacatcaaggacacctacatccactgggtgcgccaggcccccggcaagggcctggagtgggtggcccggatctaccccaccaacggctacacccgctacgccgacagcgtgaagggccggttcaccatcagcgccgacacctccaagaacaccgcctacctgcagatgaacagcctgcgcgccgaggacaccgccgtgtactactgcagccggtggggcggcgacggattctacgccatggactactggggacagggcaccctggtgaccgtgagcagcggtggcggaggatctggcggaggcggtagtggcggtggcggatctgacatccagatgacccagagcccttccagcctgagcgccagcgtgggcgaccgggtgaccatcacctgccgcgctagccaggacgtgaacaccgccgtggcctggtaccagcagaagcccggaaaggcccccaagctgctgatctactctgctagcttcctgtacagcggcgtgcccagccggttcagcggatctcgcagcggcaccgacttcaccctgaccatcagcagcctgcagcctgaggacttcgccacctactactgccagcagcactacaccacgcctcccaccttcggacagggcaccaaggtggagatcaagagcggcggaggtggttcagaggtgcagctggtggagagcggtggcggcctggtgcaacctggcggtagcttgaggttgagctgcgccgcaagcggcttcaccttcaacacctacgccatgaactgggtgcgccaggccccaggcaagggcctggagtgggtggccaggatcaggagcaagtacaacaactatgccacctactacgccgacagcgtgaaggacaggttcaccgtcagcagggacgacagcaagaacaccgcctacctgcagatgaactcactgaagaccgaggacaccgcagtgtactactgcgtgaggcatggcaacttcggcaacagctacgtgagctggttcgcctactggggccagggcaccatggtgaccgtgagcagcgtggagggcggctccgggggatcaggcggcagtggcggaagcggcggagttcagaccgtggtgacccaggagcctagcctgaccgtgagccctggcggaaccgtgacccttacctgcgcctcaagcaccggagcagttaccaccagcaactacgccaactgggtccagcagaatcccgggcaagcccccaggggcctgattggcggcaccaacaagaaggcccccgggacccccgctaggttcagcggcagcctgctgggcggcaaggccgcactgaccctgtccggagtccagcccgaggacgaggccgagtactactgcgccctgtggtacagcaacctgtgggtatttggcggtggcactaaattgaccgttctgcaccaccatcatcaccattag

SEQ ID NO: 62 - DNA, 1575 bp, Artificial Sequence
  Synthetic
atggaatggagctgggtctttctcttcttcctgtcagtaacgactggtgtccactccgaggtgcagctggtggagagcggcggaggcctcgtgcagcccggcggatctctgcggctgagctgcgccgctagcggcttcaacatcaaggacacctacatccactgggtgcgccaggcccccggcaagggcctggagtgggtggcccggatctaccccaccaacggctacacccgctacgccgacagcgtgaagggccggttcaccatcagcgccgacacctccaagaacaccgcctacctgcagatgaacagcctgcgcgccgaggacaccgccgtgtactactgcagccggtggggcggcgacggattctacgccatggactactggggacagggcaccctggtgaccgtgagcagcggtggcggaggatctggcggaggcggtagtggcggtggcggatctgacatccagatgacccagagcccttccagcctgagcgccagcgtgggcgaccgggtgaccatcacctgccgcgctagccaggacgtgaacaccgccgtggcctggtaccagcagaagcccggaaaggcccccaagctgctgatctactctgctagcttcctgtacagcggcgtgcccagccggttcagcggatctcgcagcggcaccgacttcaccctgaccatcagcagcctgcagcctgaggacttcgccacctactactgccagcagcactacaccacgcctcccaccttcggacagggcaccaaggtggagatcaagagcggcggaggtggttcagaggtgcagctggtggagagcggtggcggcctggtgcaacctggcggtagcttgaggttgagctgcgccgcaagcggcttcaccttcaacacctacgccatgaactgggtgcgccaggccccaggcaagggcctggagtgggtggccaggatcaggagcaagtacaacaactatgccacctactacgccgacagcgtgaagggcaggttcaccgtcagcagggacgacagcaagaacaccgcctacctgcagatgaactcactgaagaccgaggacaccgcagtgtactactgcgtgaggcatggcaacttcggcaacagctacgtgagctggttcgcctactggggccagggcaccatggtgaccgtgagcagcgtggagggcggctccgggggatcaggcggcagtggcggaagcggcggagttcagaccgtggtgacccaggagcctagcctgaccgtgagccctggcggaaccgtgacccttacctgcggctcaagcaccggagcagttaccaccagcaactaccccaactgggtccagcagaaacccgggcaagcccccaggggcctgattggcggcaccaacaagaaggcccccgggacccccgctaggttcagcggcagcctgctgggcggcaaggccgcactgaccctgtccggagcccagcccgaggacgaggccgagtactactgcgccctgtggtacagcaacctgtgggtatttggcggtggcactaaattgaccgttctgcaccaccatcatcaccattag

SEQ ID NO: 63 - DNA, 705 bp, Artificial Sequence
  Synthetic
atggagaccgacaccctgctgctctgggtgctgctgctctgggtgcccggctccaccggagacatccagatgacccagagcccttccagcctgagcgccagcgtgggcgaccgggtgaccatcacctgccgcgctagccaggacgtgaacaccgccgtggcctggtaccagcagaagcccggaaaggcccccaagctgctgatctactctgctagcttcctgtacagcggcgtgcccagccggttcagcggatctcgcagcggcaccgacttcaccctgaccatcagcagcctgcagcctgaggacttcgccacctactactgccagcagcactacaccacgcctcccaccttcggacagggcaccaaggtagagatcaagcggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa